(19) United States
(12) Patent Application Publication
(10) Pub. No.: US 2024/0092778 A1
(43) Pub. Date: Mar. 21, 2024

(54) TOPICAL PHARMACEUTICAL COMPOSITIONS AND METHODS

(71) Applicant: PYRAMID BIOSCIENCES, INC., Bridgewater, NJ (US)

(72) Inventors: Kollol PAL, Waltham, MA (US); Abhijit BHAT, Waltham, MA (US); Jay BIRNBAUM, Montville, NJ (US); Vijendra NALAMOTHU, Durham, NC (US); Srikanth MANNE, Durham, NC (US); Gayathri KRISHNAN, Durham, NC (US); Jean-Philippe THERRIEN, Davidson, NC (US)

(21) Appl. No.: 18/266,974

(22) PCT Filed: Dec. 28, 2021
(86) PCT No.: PCT/IB2021/062364

§ 371 Date: Jun. 13, 2023

Related U.S. Application Data
(60) Provisional application No. 63/131,647, filed on Dec. 29, 2020.

(51) Int. Cl. C07D 471/04; A61K 9/00; A61K 9/06; A61K 9/107; A61K 47/10; A61K 47/18; A61P 17/06

(52) U.S. Cl. CPC C07D 471/04; A61K 9/0019; A61K 9/06; A61K 9/1075; A61K 47/10; A61K 47/18; A61P 17/06

(57) ABSTRACT

The present disclosure relates to a topical pharmaceutical composition containing N-[(1S)-1-(5-fluoropyrimidin-2-yl)ethyl]-3-(5-isopropoxy-1H-pyrazol-3-yl)-3H-imidazo[4,5-b]pyridin-5-amine (hereinafter also referred to as “Compound A”), or a pharmaceutically acceptable salt thereof; to the use of the topical pharmaceutical composition as a medicament; to processes for the preparation of said topical pharmaceutical composition; to certain new methods of treating an inflammatory skin disorder, particularly psoriasis, by administering a topical pharmaceutical composition containing Compound A or a pharmaceutically acceptable salt thereof; and to novel crystalline forms of a mesylate salt of Compound A.

### CROSS-REFERENCE TO RELATED APPLICATION

This application is an international application filed under PCT Article 10 claiming priority to U.S. Ser. No. 63/131,647, filed Dec. 29, 2020, which is incorporated by reference in its entirety.

### TECHNICAL FIELD

The present disclosure relates to a topical pharmaceutical composition containing N-[(1S)-1-(5-fluoropyrimidin-2-yl)ethyl]-3-(5-isopropoxy-1H-pyrazol-3-yl)-3H-imidazo[4,5-b]pyridin-5-amine (hereinafter also referred to as “Compound A”), or a pharmaceutically acceptable salt thereof, to the use of the topical pharmaceutical composition as a medicament; to processes for the preparation of said topical pharmaceutical composition; to certain new methods of treating an inflammatory skin disorder, particularly psoriasis, by administering a topical pharmaceutical composition containing Compound A or a pharmaceutically acceptable salt thereof; and to novel crystalline forms of a mesylate salt of Compound A.

### BACKGROUND

Growth factors are important signaling molecules that promote the growth, development and homeostasis of many cellular systems. Neurotrophins are growth factors which are responsible for central and peripheral neuronal growth, maturation and death. Neurotrophins activate cell surface receptors called tropomyosin-like receptors which in turn regulate intracellular kinases called tyrosine receptor kinases. The tropomyosin-related kinase (TRK) family of receptors includes TRKA, TRKB, TRKC and p75 and serve as high affinity cell surface receptors for the growth factors NGF, BDNF, NT3 and NT4, respectively. Inhibition of these receptors may lead to the modulation or inhibition of intracellular signaling cascades that regulate cell growth and proliferation, cellular communication between cells that regulate signaling, feedback mechanism and homeostasis. These growth factors have been implicated in the growth and proliferation of both neuronal and non-neuronal cells.

TRK inhibitors have the potential to be used in the treatment or prevention of various diseases including skin disorders, pain, inflammatory and immunological conditions. For example, neurotrophins are involved in the regulation of skin homeostasis, skin remodeling and response to various pathological conditions including infections, inflammation and other skin insults. Specifically, neurotrophins have been implicated in many dermatoses including atopic dermatitis, psoriasis, alopecia, mastocytosis, actinic keratosis and cylindromatosis (see, e.g., Truzzi, et. al. (2011) Dermato-Endocrinology 3: 32; Botchkarev, et. al. (2006) J. Investigative Dermatology 126:1719; Papoui, et. al. (2011) Neuropeptides, 45: 417). Compounds that can block the neurotrophin signaling cascade have been demonstrated to be beneficial in the treatment of skin disorders which rely on TRK signaling (see, e.g., Raychaudhuri, et. al. (2004) J. Investigative Dermatology 122:812; Raychauduri, et. al. (2004) Prog. Brain Res, 146: 133; Cranston, et. al. (2017) Trials, 18: 111). Thus, a number of TRK inhibitors have been shown to have potential for the treatment of dermatoses.

N-[(1S)-1-(5-fluoropyrimidin-2-yl)ethyl]-3-(5-isopropoxy-1H-pyrazol-3-yl)-3H-imidazo[4,5-b]pyridin-5-amine, referred to as Compound A, is a potent and highly specific TRK inhibitor. The compound is disclosed and its synthesis described in International Patent Application WO 2008/135785. Compound A has the following chemical structure:

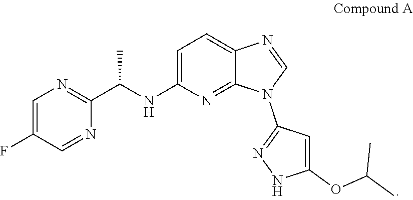 [SMILES: CC(C)Oc1cc(-n2cnc3ccc(N[C@@H](C)c4ncc(F)cn4)nc32)n[nH]1]

The Applicants have discovered that Compound A or a pharmaceutically acceptable salt thereof can be used for the treatment of certain dermatoses when applied topically.

A topical pharmaceutical composition can allow an active ingredient to be directly administered to diseased skin and can prove to be beneficial in treating such diseases. However, in order for a composition to be suitable for use as a medicament, the composition needs to exhibit certain characteristics. For example, the dosage form should provide the necessary type of drug release and permeation in order to enable the active drug compound to access and be retained (at the required levels) at the desired site of action within the skin while limiting the amount of drug that penetrates the underlying tissue and becomes systemically available. This balance can be difficult to achieve as the main function of the skin is to limit the exchange of substances between the body and the environment. The skin barrier presents a challenge for the penetration and retention of an active ingredient at the desired target. Additionally, the active ingredient needs to be chemically and physically stable within the composition and the composition itself also needs to be stable. For example, for an emulsion-based composition, such as a cream, phase separation needs to be avoided in order to maintain physical stability throughout the shelf-life of the product. Finally, it is also important that the composition is well tolerated upon application and continued use.

This is a particularly challenging mix of problems to address for a topical formulation of Compound A. There is, therefore, a need for new topical pharmaceutical compositions containing Compound A, or a pharmaceutically acceptable salt thereof, which provide suitable release, permeation and stability and are also well tolerated upon application and continued use.

As mentioned above, neurotrophins have been implicated in skin disorders including atopic dermatitis, psoriasis, alopecia, mastocytosis, actinic keratosis and cylindromatosis. However, no TRK inhibitor has been found to be sufficiently efficacious for the treatment of inflammatory skin disorders, such as Psoriasis.

Psoriasis is a multisystem, chronic inflammatory disease predominantly affecting skin and joints. Sustained inflammation leads to uncontrolled keratinocyte proliferation and dysfunctional differentiation, resulting in lesions in the outermost layer of the skin (Rendon et. al. (2019) Int J Mol Sci. 2019. 20(6):1475). Despite its high prevalence, approximately 10 million people in the United States and 125 million people worldwide, psoriasis is currently underdiagnosed and undertreated (Armstrong et. al. (2020) JAMA 323(19):1945-1960). It affects both men and women equally, and adults more often than children. A disease of systemic inflammation, the manifestations of this disease are not limited to the skin but also affect joints and tendons. Psoriatic arthritis develops in up to 40% of subjects with psoriasis, with dermatological symptoms generally occurring first. Psoriasis is also associated with other comorbidities including cancer, cardiometabolic disease, and mental health disorders, substantially impacting quality of life.

Compound A has only ever clinically been evaluated in a Phase 1 trial in adults with recurrent glioblastoma multiforme (GBM). A summary of the study results was presented in Kreisl et. al. (2014) Neuro-Oncology 16:v79-v95. In this study, up to 60 mg of the compound was administered daily. No early efficacy signals in GBM patients were observed and the drug program was subsequently discontinued by the clinical trial sponsor, AstraZeneca Pharmaceuticals.

In 2015, it was reported that an investigational topical selective TRKA inhibitor, CT327, had been dosed in a Phase 2b clinical trial in patients with psoriasis (Roblin et. al. (2015) Acta Derm Venereol 2015; 95: 542-548). According to the report, no effect was found on psoriasis severity and no significant improvement in controlled disease response was seen for any dose of CT327 compared to vehicle. The authors concluded that the inhibition of keratinocyte hyperproliferation and sensory neuron function via TRKA kinase in the study may not be sufficient to alter the entire psoriatic disease state over 8 weeks of dosing, as measured by changes in either the IGA or mPASI instruments. The report thus teaches away from further investigation of selective TRKA inhibitors, such as CT327, for the treatment of psoriasis.

In addition to providing efficacy for inflammatory skin disorders, such as psoriasis, such treatments also need to exhibit a suitable safety profile. The Applicant has surprisingly found that certain dosages of Compound A applied topically to the skin can be beneficial with regards to treatment of inflammatory skin disorders, such as psoriasis.

### SUMMARY

In order for certain topical pharmaceutical compositions to deliver an active compound in a manner in which it can engage at the site of action, e.g., an epidermal target, sufficient levels of the active ingredient need to permeate the stratum corneum into the viable epidermis and need to be retained in this tissue layer for a sufficient period of time. The skin barrier makes the penetration and retention of an active ingredient a challenge. In this regard, the Applicants faced particular challenges when developing a topical pharmaceutical composition containing Compound A.

Furthermore, a topical pharmaceutical composition must be chemically and physically stable during manufacture and upon storage. For example, the active ingredient must be physically and chemically stable within the topical pharmaceutical composition. Additionally, the topical pharmaceutical composition itself must remain chemically and physically stable, for example, no or minimal discoloration should be present and/or no segregation of components should occur during manufacture and upon storage. Furthermore, if the composition is an emulsion-based composition, then phase separation must be avoided during the shelf-life of the composition. Additionally, if the active ingredient is dissolved within the topical pharmaceutical composition it must ideally remain dissolved throughout the product shelf-life of the dosage form.

Another important consideration when developing a topical pharmaceutical formulation is that upon application to the skin of a patient, no or minimal local side effects must occur, for example skin irritation, redness, erythema, and itching. Skin irritation can occur if the pH of the topical pharmaceutical composition is outside the normal range of the pH of skin and does not adjust upon application. Typically, the skin has a pH of around 5.5-6.5. It is important for the topical pharmaceutical composition to not cause local side effects upon administration for a number of reasons. For example, such irritation can aggravate the condition being treated, cause discomfort to the patient, and hinder patient compliance (a patient is more likely to continue administering the topical pharmaceutical composition if no local side effects occur).

Applicants have surprisingly found topical pharmaceutical compositions containing Compound A, or pharmaceutically acceptable salts thereof, which have adequate permeability properties, good stability profiles (chemically and physically) and are well tolerated upon application.

In a first aspect, the present disclosure provides a topical pharmaceutical composition comprising:
a) Compound A or a pharmaceutically acceptable salt thereof.

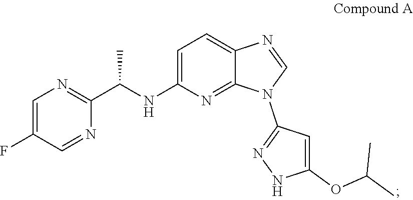 [SMILES: CC(C)Oc1cc(-n2cnc3ccc(N[C@@H](C)c4ncc(F)cn4)nc32)n[nH]1]

b) at least one permeation enhancer; and
c) one or more pharmaceutically acceptable excipients.

The permeation enhancer is required to achieve adequate permeation of Compound A, or a pharmaceutically acceptable salt thereof, into the epidermal layer of the skin. In some embodiments, a permeation enhancer can advantageously enhance permeation and also help solubilize Compound A or a pharmaceutically acceptable salt thereof in the composition without causing too much penetration so as to avoid undesired systemic exposure in the circulation.

Applicants found that the need for a permeation enhancer in the composition led to further unexpected technical difficulties. It was found that certain permeation enhancers, which were regarded as particularly advantageous because they were also able to help solubilize Compound A or a pharmaceutically acceptable salt thereof in the composition, caused significant degradation of Compound A upon manufacture and/or storage. Through additional investigations and research, Applicants found that when Compound A, or a pharmaceutically acceptable salt thereof, is formulated with certain permeation enhancers, antioxidants were useful to inhibit chemical degradation and provide a suitable stability profile. Surprisingly, the Applicants found that only certain types of antioxidants, in particular oil-soluble antioxidants, provided the best stability profiles for certain permeation enhancers.

In addition to the technical difficulties described above, the Applicants also found that when Compound A was formulated as a salt, such as the mesylate salt, the pH of the topical pharmaceutical composition dropped to a level in the range of about pH 2, which consequently resulted in skin irritation upon application and instability of the composition itself. The Applicants investigated a number of different buffering systems with the aim of finding a system that adjusted and subsequently maintained the pH of the topical pharmaceutical composition to a more suitable value that would be compatible with the skin, approximately pH 5.5. Surprisingly, only certain buffering systems tested provided the preferred desired pH properties.

The present disclosure further provides a process for preparing a topical pharmaceutical composition, the process comprising the steps of:
a) providing Compound A or a pharmaceutically acceptable salt thereof in an aqueous phase, wherein the aqueous phase optionally also comprises at least one hydrophilic emulsifier (for example, polysorbate such as polysorbate 80) and/or at least one preservative and/or at least one chelating agent to provide a homogenous aqueous phase;
b) further providing at least one permeation enhancer in the aqueous phase;
c) emulsifying the aqueous phase at an elevated temperature into an oil phase to form an emulsion, then allowing the emulsion to cool to form a topical composition (such as a cream or ointment), wherein the oil phase comprises at least one emollient, at least one emulsifier and at least one antioxidant and optionally also comprises at least one preservative, at least one further permeation enhancer and at least one counter-irritant; and
d) optionally adjusting the pH of the composition by incorporating a pH-adjusting agent.

The present disclosure further provides a process for preparing a topical pharmaceutical composition, the process comprising the steps of:
a) providing a Compound A or a pharmaceutically acceptable salt thereof in an aqueous phase;
b) providing a further aqueous phase comprising at least one hydrophilic emulsifier (e.g. polysorbate, for example polysorbate 80) and optionally at least one preservative and/or at least one chelating agent to provide a homogenous aqueous phase;
c) further providing at least one permeation enhancer in the aqueous phase a) or b);
d) emulsifying the aqueous phase of a) and b) at an elevated temperature into an oil phase to form an emulsion, then allowing the emulsion to cool to form a topical composition (such as a cream or ointment), wherein the oil phase comprises at least one emollient, at least one emulsifier and at least one antioxidant and optionally also comprises at least one preservative, at least one further permeation enhancer and at least one counter-irritant; and
e) optionally adjusting the pH of the composition by incorporating a pH-adjusting agent.

In some embodiments there is provided a product obtainable by any of the processes described herein.

The present disclosure also provides a method for treating a disease or condition mediated alone or in part by TRK, such as a skin disorder, by administration to a subject in need thereof a topical pharmaceutical composition according to the first aspect.

The present disclosure also provides a composition for use in treating a disease or condition mediated alone or in part by TRK, such as a skin disorder, by administration to a subject in need thereof a topical pharmaceutical composition according to the first aspect.

The applicant has also advantageously found a dosing range of Compound A, or a pharmaceutically acceptable salt thereof, which is particularly useful for treating or preventing inflammatory skin disorders, such as psoriasis. There is therefore provided a method of treating or preventing an inflammatory skin disorder comprising administration of a topical pharmaceutical composition to a subject in need thereof, wherein the topical pharmaceutical composition comprises Compound A:

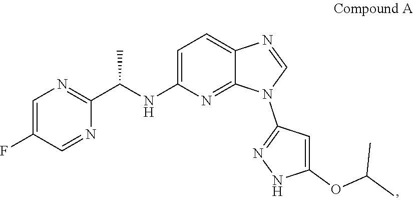 [SMILES: CC(C)Oc1cc(-n2cnc3ccc(N[C@@H](C)c4ncc(F)cn4)nc32)n[nH]1]

or a pharmaceutically acceptable salt thereof;
and wherein the composition is:
   a) applied topically to an affected area of the skin of a subject at an application dose of Compound A or a pharmaceutically acceptable salt thereof in the range of 0.05 μg/cm² to 1000 μg/cm² skin surface; and
   b) the composition is administered in an amount sufficient to prevent or treat the inflammatory skin disorder in the subject.

The present disclosure further provides a composition for use in treating or preventing an inflammatory skin disorder comprising administration of a topical pharmaceutical composition to a subject in need thereof, wherein the topical pharmaceutical composition comprises Compound A:

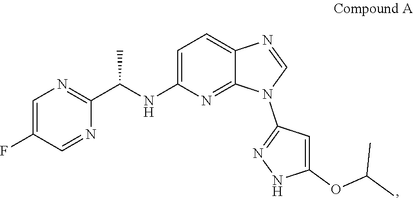 [SMILES: CC(C)Oc1cc(-n2cnc3ccc(N[C@@H](C)c4ncc(F)cn4)nc32)n[nH]1]

or a pharmaceutically acceptable salt thereof;
and wherein the composition is:
   c) applied topically to an affected area of the skin of a subject at an application dose of Compound A or a pharmaceutically acceptable salt thereof in the range of 0.05 μg/cm² to 1000 μg/cm² skin surface; and
   d) the composition is administered in an amount sufficient to prevent or treat the inflammatory skin disorder in the subject.

The present disclosure further provides certain novel crystalline forms of a mesylate salt of Compound A.

### BRIEF DESCRIPTION OF THE DRAWINGS

FIG. 1A is the XRPD diffractogram of Compound A mesylate salt Form B.

FIG. 1B is the XRPD diffractogram of Compound A mesylate salt Form A.

FIG. 2 is a graph showing the ET-50 data and irritancy classification of the exemplary formulations.

FIG. 3 is a graph showing the IL-1α data of the exemplary formulations.

FIG. 4 is a graph showing the proliferation assay in primary human keratinocytes treated with Compound A mesylate salt after 24 hours and 72 hours treatment.

FIG. 5 is a graph showing the apoptosis assay using Annexin V read-out in primary human keratinocytes treated with Compound A mesylate salt after 24 h and 72 h treatment.

FIG. 6 is a graph showing the cell death assay using propidium iodide read-out in primary human keratinocytes treated with Compound A mesylate salt after 24 h and 72 h treatment.

### DETAILED DESCRIPTION

In a first aspect, the present disclosure provides a topical pharmaceutical composition comprising:
a) Compound A or a pharmaceutically acceptable salt thereof:

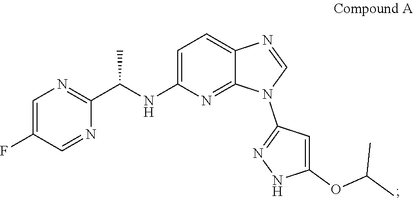 [SMILES: CC(C)Oc1cc(-n2cnc3ccc(N[C@@H](C)c4ncc(F)cn4)nc32)n[nH]1]

b) at least one permeation enhancer; and
c) one or more pharmaceutically acceptable excipients.

The disclosed compositions, processes of manufacture, methods, and novel crystalline forms of the mesylate salt may be understood more readily by reference to the following detailed description.

### Definitions

It is to be understood that concentrations, amounts, and other numerical data may be expressed or presented herein in range formats. It is to be understood that such range formats are used merely for convenience and brevity and include not just the numerical values explicitly recited as the end points of the range but also to include all the individual numerical values encompassed within that range as if each numerical value is explicitly recited. As an illustration, a numerical range of “1% w/w to 5% w/w” should be interpreted to include not just the explicitly recited values of 1% w/w to 5% w/w, but also include individual values within the indicated range. Thus, included in this numerical range are individual values such as 2, 3.5, and 4% w/w, etc. All ranges are inclusive and combinable.

It is to be appreciated that certain features of the disclosed compositions, processes of manufacture and methods which are, for clarity, described herein in the context of separate embodiments, may also be provided in combination in a single embodiment. Conversely, various features of the disclosed compositions, processes of manufacture and methods that are, for brevity, described in the context of a single embodiment, may also be provided separately or in any sub-combination.

The term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

As used herein, “treating” and like terms refer to reducing the severity and/or frequency of symptoms, eliminating symptoms and/or the underlying cause of said symptoms, reducing the frequency or likelihood of symptoms and/or their underlying cause, delaying, preventing the recurrence and/or slowing the progression of diseases and/or disorders, such as proliferative skin disorders, and improving or remediating damage caused, directly or indirectly, by the diseases and/or disorders such as cancers or benign proliferative disorders.

As used herein, the phrase “therapeutically effective dose” or “therapeutically effective amount” refers to an amount of a composition comprising at least Compound A or a pharmaceutically acceptable salt thereof, as described herein, effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results include, but are not limited to, the reduction, remission, and/or regression of the benign or malignant disease or prevention of the development of the benign or malignant disease, as determined by any means suitable in the art.

Unless otherwise stated, the term “soluble”, as used herein means the solute has a solubility, e.g. in the solvent or permeation enhancer, of at least 2 mg/g when measured at 20±2° C., such as at least 4 mg/g.

The term “subject”, as used herein, includes humans, as well as non-human subjects such as cats, dogs, sheep, cattle, pigs, goats, non-human primates (including monkeys and apes), and the like. Conveniently, the subject is a human.

The term “pharmaceutically acceptable excipient” is used herein to refer to an essentially pharmacologically inert, non-toxic substance, e.g., that has been approved for inclusion in pharmaceutical products.

### Compound A

Compound A, as referred to in the present disclosure, is N-[(1S)-1-(5-fluoropyrimidin-2-yl)ethyl]-3-(5-isopropoxy-1H-pyrazol-3-yl)-3H-imidazo[4,5-b]pyridin-5-amine:

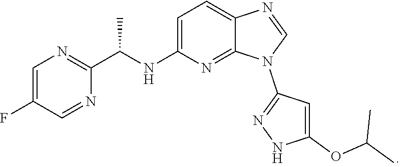 [SMILES: CC(C)Oc1cc(-n2cnc3ccc(N[C@@H](C)c4ncc(F)cn4)nc32)n[nH]1]

As depicted above, Compound A is shown as a “free base”. In some embodiments, a pharmaceutically acceptable salt of Compound A is used.

Pharmaceutically acceptable salts of Compound A include acid addition salts formed with inorganic acids or organic acids. Examples of acid addition salts include, but are not limited to, acetate, adipate, ascorbate, benzoate, besylate (benzenesulfonate), bicarbonate, bisulfate, butyrate, camphorate, camphorsulfonate, citrate, cyclohexyl sulfamate, esylate (ethanesulfonate), fumarate, glutamate, glycolate, hemisulfate, 2-hydroxyethylsulfonate, heptanoate, hexanoate, hydrochloride, hydroiodide, hydromaleate, lactate, maleate, mesylate (methanesulfonate), meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, undecanoate, and 2,5-dimethylbenzene sulfonate.

In some embodiments, a pharmaceutically acceptable salt of Compound A is selected from besylate, esylate, fumarate, hydrochloride, maleate, mesylate, 2-naphthalenesulfonate, nitrate, tosyalte, succinate, sulfate, and 2,5-dimethylbenzene sulfonate.

In some embodiments a pharmaceutically acceptable salt of Compound A is selected from besylate, esylate, hydrochloride, mesylate, and tosylate.

In some embodiments the pharmaceutically acceptable salt of Compound A is a mesylate salt. The mesylate salt of Compound A is depicted below:

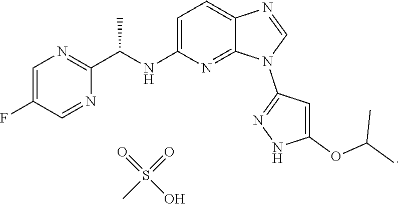

The molecular weight of Compound A is 382.4 g/mol. In some embodiments the molar ratio of Compound A to mesylate in the salt form is 1:1. The molecular weight of the 1:1 mesylate salt of Compound A is 478.5 g/mol.

Throughout the specification, unless specified otherwise, references to the amount of Compound A will be understood to refer to the amount of the parent compound on a free base basis, even if the compound is present as a salt of Compound A. Purely by way of example, reference to 10 mg of Compound A or a salt thereof, will be understood to refer to 10 mg of the free base, or a salt of Compound A with 10 mg of free base equivalent. In the context of the mono-mesylate salt of Compound A, 12.5 mg of the salt delivers 10 mg of Compound A (free base equivalent).

It is to be understood that, insofar as Compound A or a pharmaceutically acceptable salt may exist in tautomeric forms, the disclosure includes in its definition any such tautomeric form which possesses the herein mentioned activity/activities. Thus, the disclosure relates to all tautomeric forms of Compound A or a pharmaceutically acceptable salt thereof which inhibit TRK related activities in a human or animal.

It is also to be understood that Compound A or a pharmaceutically acceptable salt thereof may exist in solvated as well as un-solvated forms such as, for example, hydrated forms. It is to be understood that the disclosure encompasses all such solvated or un-solvated forms.

Additionally, unless otherwise stated, structures depicted herein are also meant to include compounds that differ only in the presence of one or more isotopically enriched atoms. For example, compounds having the present structures including the replacement of hydrogen by deuterium are within the scope of this disclosure.

It is also to be understood that Compound A or a pharmaceutically acceptable salt thereof may exhibit polymorphism, and that the disclosure encompasses all such forms that possess TRK activity. As used herein, the term “crystal polymorphs”, “polymorphs”, or “crystal forms” means crystal structures in which a compound (or a salt or solvate/hydrate thereof) can crystallize in different crystal packing arrangements, all of which have the same elemental composition. Different crystal forms usually have different X-ray diffraction patterns, infrared spectra, melting points, density hardness, crystal shape, optical and electrical properties, stability and solubility. Recrystallization solvent, rate of crystallization, storage temperature, and other factors may cause one crystal form to dominate. Crystal polymorphs of the compounds, or pharmaceutically acceptable salts thereof, can be prepared by crystallization under different conditions.

The mesylate salt of Compound A exhibits polymorphism. Reference to Compound A or a pharmaceutically acceptable salt thereof (e.g., the mesylate salt of Compound A) includes one or more polymorphs unless otherwise stated.

In some embodiments, there is provided a crystalline polymorph of the mesylate salt of Compound A, suitably Form B. Advantageously, Form B is to-date the most thermodynamically stable polymorphic form of the mesylate salt of Compound A identified. Form B has a high melting point in the range of 230 to 250° C., such as in the range of 240 to 244° C., such as about 242° C.

In some embodiments, the mesylate salt of Compound A Form B exhibits an X-ray diffraction pattern summarised by Table 1A.

In some embodiments, provided herein is a crystalline polymorph of the mesylate salt of Compound A comprising at least 3 peaks (±0.2° 2θ or conveniently ±0.1° 2θ) of Table 1A, such as at least 4 peaks, 5 peaks, 6 peaks, 7 peaks, 8 peaks, 9 peaks, or 10 peaks (0.2° 2θ or conveniently ±0.1° 2θ).

TABLE 1A

| 2θ (Theta) | Relative |
|---|---|
| value | Intensity % |
| 6.44 | 100 |
| 7.28 | 6.83 |
| 9.17 | 2.67 |
| 12.89 | 2.4 |
| 13.47 | 3.07 |
| 14.60 | 10.43 |
| 16.24 | 9.56 |
| 17.19 | 5.24 |
| 17.42 | 2.33 |
| 18.41 | 6.71 |
| 19.40 | 40.24 |
| 19.95 | 10.53 |
| 20.48 | 26.04 |
| 20.72 | 20.65 |
| 21.88 | 6.53 |
| 22.74 | 12.41 |
| 23.48 | 5.24 |
| 24.30 | 8.98 |
| 25.45 | 3.46 |
| 25.96 | 8.19 |
| 26.35 | 1.88 |
| 26.8 | 4.11 |
| 27.8 | 15.52 |
| 29.4 | 2.96 |
| 29.84 | 2.55 |
| 32.79 | 1.65 |
| 33.51 | 3.39 |

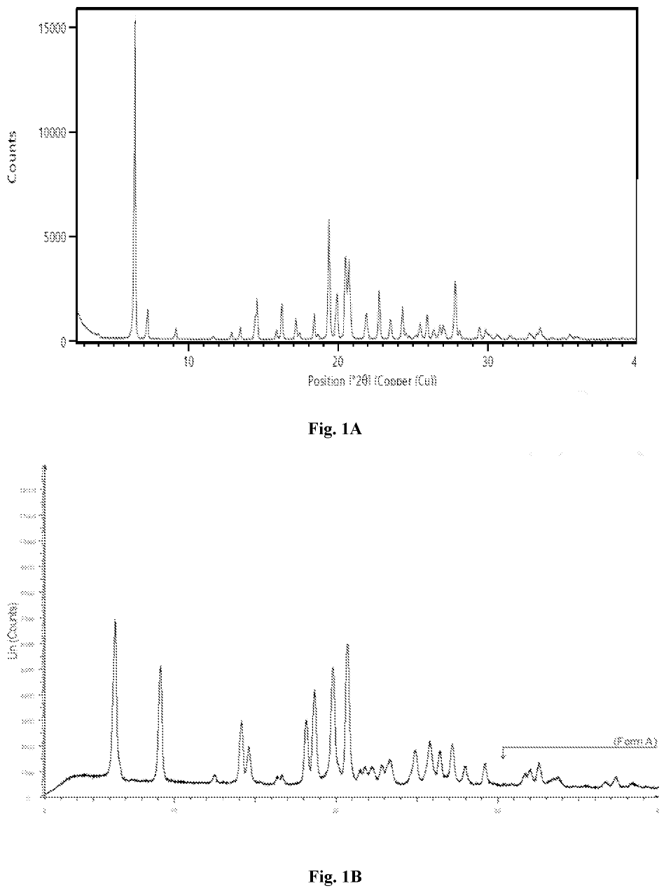

In some embodiments, there is provided the crystalline polymorph Form B of the mesylate salt of Compound characterised by an X-ray diffraction pattern comprising peaks at 6.44, 14.60, and 19.40° 2θ+0.2° 2θ (conveniently °2θ+0.1° 2θ). In some embodiments the crystalline polymorph Form B is further characterised by at least one peak appearing at 7.28, 16.24, 17.19, 18.41, or 19.95° 2θ±0.2° 2θ (conveniently °2θ±0.1°2θ). In some embodiments the crystalline polymorph Form B is further characterised by at least two peaks appearing at 7.28, 16.24, 17.19, 18.41, or 19.95° 2θ+0.2° 2θ (conveniently 2θ+0.1° 2θ). In some embodiments the crystalline polymorph Form B exhibits an X-ray diffraction powder pattern substantially similar to, or the same as, the X-ray powder diffraction pattern shown in FIG. 1A.

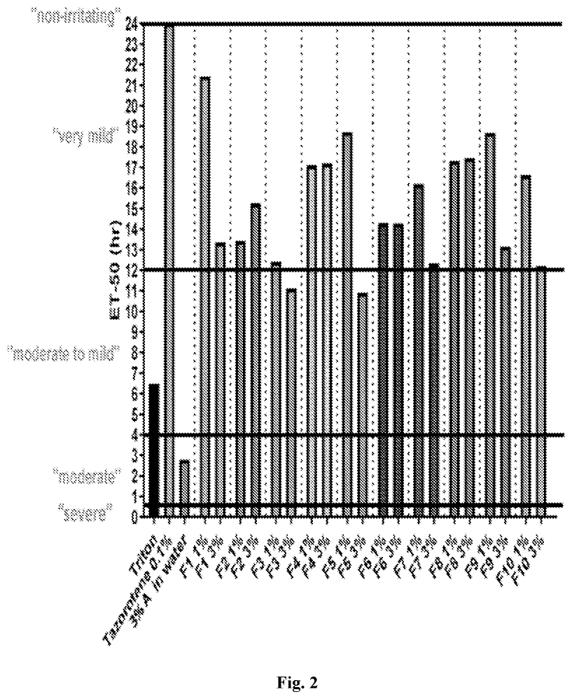

The X-ray powder diffraction pattern in FIG. 1A and 2θ values according to Table 1A were generated from an X-ray powder diffractometer using Cu Ka radiation (1.54060 Å).

In some embodiments, provided herein is a pharmaceutical composition comprising a therapeutically effective dose of a disclosed crystalline polymorph and a pharmaceutically acceptable carrier.

In some embodiments, there is provided a crystalline polymorph of the mesylate salt of Compound A, suitably Form A. Form A has a high melting point of about 238° C.

In some embodiments, the mesylate salt of Compound A Form A exhibits an X-ray diffraction pattern summarised by Table 1B.

In some embodiments, provided herein is a crystalline polymorph of the mesylate salt of Compound A Form A comprising at least 3 peaks (±0.2° 2θ or conveniently ±0.1°2θ) of Table 1B, such as at least 4 peaks, 5 peaks, 6 peaks, 7 peaks, 8 peaks, 9 peaks, or 10 peaks (0.2° 2θ or conveniently 0.1°2θ).

TABLE 1B

| 2θ (Theta) | Relative |
|---|---|
| value | Intensity % |
| 6.1 | 100 |
| 6.42 | 5.04 |
| 8.9 | 8.53 |
| 12.27 | 2.18 |
| 13.91 | 6.01 |
| 14.39 | 1.74 |
| 16.14 | 2.76 |
| 16.52 | 2.14 |
| 17.98 | 2.61 |
| 18.49 | 18.10 |
| 19.6 | 13.38 |
| 20.52 | 8.64 |
| 21.28 | 1.16 |
| 22.13 | 1.07 |
| 22.69 | 2.11 |
| 24.75 | 5.66 |
| 25.61 | 4.38 |
| 26.29 | 1.62 |
| 27.00 | 1.97 |
| 27.8 | 2.48 |

In some embodiments, there is provided the crystalline polymorph Form A of the mesylate salt of Compound characterised by a X-ray diffraction pattern comprising peaks at 6.1, 8.9, and 13.91° 2θ+0.2° 2θ (conveniently 2θ±0.1° 2θ). In some embodiments the crystalline polymorph Form B is further characterised by at least one peak appearing at 12.27, 16.14, 19.6, 18.41, or 20.5° 2θ+0.2° 2θ (conveniently 2θ±0.1° 2θ). In some embodiments the crystalline polymorph Form B exhibits an X-ray diffraction powder pattern substantially similar to, or the same as the X-ray powder diffraction pattern shown in FIG. 1B.

The X-ray powder diffraction pattern in FIG. 1B and °2θ theta values according to Table 1B were generated from an X-ray powder diffractometer using Cu Ka radiation (1.54060 Å).

In some embodiments, provided herein is a pharmaceutical composition comprising a therapeutically effective dose of a disclosed crystalline polymorph and a pharmaceutically acceptable carrier.

In some embodiments, the topical pharmaceutical composition comprises from about 0.005% w/w to about 5% w/w of Compound A or a pharmaceutically acceptable salt thereof, such as from about 0.005% w/w to about 3% w/w, from about 0.005% to about 0.3% w/w, from about 0.005% w/w to about 0.15% w/w, or from about 0.01% w/w to about 0.15% w/w.

In some embodiments, the topical pharmaceutical composition comprises between 0.005% w/w and 5% w/w of Compound A or a pharmaceutically acceptable salt thereof, such as between 0.005% w/w and 3% w/w, between 0.005% and 0.3% w/w, between 0.005% w/w and 0.15% w/w, or between 0.01% w/w and 0.15% w/w.

In some embodiments, the topical pharmaceutical composition comprises about 0.03% w/w of Compound A or a pharmaceutically acceptable salt thereof.

In some embodiments, the topical pharmaceutical composition comprises about 0.01% w/w of Compound A or a pharmaceutically acceptable salt thereof.

In some embodiments, the topical pharmaceutical composition comprises about 0.1% w/w of Compound A or a pharmaceutically acceptable salt thereof.

The synthesis of Compound A may be readily achieved by the methods described in PCT publication number WO2008/135785 the teachings of which are incorporated herein by reference in its entirety.

### Topical Pharmaceutical Compositions

Topical pharmaceutical compositions are compositions that are applied directly to an external body surface, which includes the skin and membranes. In order for a topical composition containing an active pharmaceutical ingredient to be successfully used as a medicament, the composition once applied to an external body surface must remain at the site of application for a sufficient period of time in order for an efficacious dose of the active pharmaceutical ingredient to be delivered to the site of treatment.

One aspect of the disclosure relates to a pharmaceutical composition comprising a therapeutically effective amount of a compound having a structure represented by a formula:

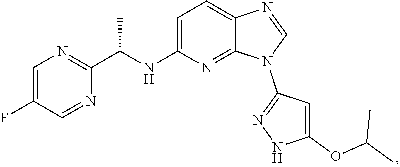 [SMILES: CC(C)Oc1cc(-n2cnc3ccc(N[C@@H](C)c4ncc(F)cn4)nc32)n[nH]1]

and a topically acceptable carrier. In a further aspect, the composition is a topical composition. In a further aspect, the composition further comprises a permeation enhancer as further described herein. In a further aspect, the composition further comprises a pH-adjusting agent as further described herein.

Topical compositions may be presented in a number of formats. Suitable formats include, but are not limited to, ointments, creams, pastes, gels, solutions, lotions, foams, sprays, transdermal patches, adhesive strips, and solid or semi-solid stick compositions. In some embodiments, the topical pharmaceutical composition is a cream, an ointment, a paste, a solution, a lotion, a gel, a foam, a spray, a rigid foam, a transdermal patch, an adhesive strip, or a solid or semi-solid stick composition, e.g., chap-stick.

Conveniently, the topical pharmaceutical composition is a cream, an ointment, a paste, a solution, a lotion, a gel, a foam or a spray.

In some embodiments, the topical pharmaceutical composition is a cream.

In some embodiments, the topical pharmaceutical composition is a cream based on an oil-in-water emulsion.

In some embodiments, the topical pharmaceutical composition is a cream based on an oil-in-water emulsion and the droplet size of the oil ranges from about 1 μm to about 50 μm, about 1 μm to about 40 μm, about 1 μm to about 30 μm, about 1 μm to about 20 μm, about 1 μm to about 10 μm, about 1 μm to about 5 μm, about 10 μm to about 50 μm, about 20 μm to about 50 μm, about 30 μm to about 50 μm, about 40 μm to about 50 μm, about 10 μm to about 40 μm, or about 20 μm to about 30 μm. In some embodiments, the droplet size of the oil ranges from about 1 μm to about 30 μm.

In some embodiments, Compound A, or a pharmaceutically acceptable salt thereof, is dissolved in the topical pharmaceutical composition. In some embodiments Compound A, or a pharmaceutically acceptable salt thereof, is partially dissolved in the topical pharmaceutical composition. In some embodiments Compound A, or a pharmaceutically acceptable salt thereof, is dispersed in the topical pharmaceutical composition. In some embodiments Compound A, or a pharmaceutically acceptable salt thereof, is partially dispersed in the topical pharmaceutical composition. Unless otherwise stated, “dissolved” means, with respect to Compound A, or a pharmaceutically acceptable salt thereof, solubilisation in the topical pharmaceutical composition such that, for example, there are no particulates of Compound A, or a pharmaceutically acceptable salt thereof, present in the topical pharmaceutical composition. The skilled person would be able to ascertain whether Compound A, or a pharmaceutically acceptable salt thereof, is dissolved by eye or using microscopy. In some embodiments, at least 0.005% w/w of Compound A, or a pharmaceutically acceptable salt is dissolved in the topical pharmaceutical composition, such as at least 0.01% w/w. Unless otherwise stated, “dispersed” means with respect to Compound A, or a pharmaceutically acceptable salt thereof, not being dissolved in the topical pharmaceutical composition such that, for example, particulates of Compound A, or a pharmaceutically acceptable salt thereof, are distributed throughout the topical pharmaceutical composition. Unless otherwise stated, “partially dissolved” or “partially dispersed” means, with respect to Compound A or a pharmaceutically acceptable salt thereof, between 50 and 95% by weight of Compound A, or a pharmaceutically acceptable salt thereof, is dissolved or dispersed in the topical pharmaceutical composition, such as at least 50% by weight, at least 60% by weight, at least 70% by weight, or such as less than 95% by weight, less than 85% by weight, or less than 75% by weight.

In some embodiments, the topical pharmaceutical composition is a cream wherein Compound A, or a pharmaceutically acceptable salt thereof, is dissolved in the cream composition.

In some embodiments, the topical pharmaceutical composition is a cream wherein Compound A, or a pharmaceutically acceptable salt thereof, is dispersed in the cream composition. In some embodiments, the topical pharmaceutical composition is a cream wherein Form B of the mesylate salt of Compound A is dispersed (or partially dispersed) in the cream composition.

In some embodiments, the topical pharmaceutical composition is a cream wherein the pH of the cream is in the range of about 3.5 to about 7.0, or in the range of about 4.5 to about 6.5 when measured at 25±1° C.

In some embodiments, the composition is a cream and has an overall hydrophilic-lipophilic balance (HLB) value in the range of about 7 to about 12, conveniently about 7 to about 9. In some embodiments, the composition is a cream and has an overall HLB value of about 8.5±0.2. It has been found by the Applicants that an optimal HLB range or value for a topical composition comprising Compound A, or a pharmaceutically acceptable salt thereof, can be achieved by careful mixture of particular components of the composition.

It is to be understood that an overall HLB for a dosage form can be calculated by taking into account the individual HLB values of the components, e.g., surfactants and emulsifiers, which themselves can be characterized according to their HLB. HLB values are commonly used to define emulsifiers and/or surfactants and refer to the hydrophilic-lipophilic balance of the given compound. HLB values can be calculated according to the methods of Griffin [Griffin, J. Soc. Cosmetic Chem. (1949), 311-326; Griffin, J. Soc. Cosmetic Chem. (1954), 249-256] as follows:

HLB=20×(MW-H/MW-T)

wherein MW-H is the molecular weight of the hydrophilic portion of the compound and MW-T is the molecular weight of the total compound. For example, for the emulsifier PEG100 stearate, MW-H is the molecular weight of the ethylene glycol portions of the molecule which is 100×44 (MW ethylene oxide monomer=44 g/mol)=4400. Stearic acid has a molecular weight of 284.5 g/mol, so MW-T=4684.5. Therefore, the HLB value for PEG100 stearate is calculated at 18.8. PEG-80 sorbitan monooleate (sold as Tween® 80 or Polysorbate 80) has a HLB value of 15.

In some embodiments, the topical pharmaceutical composition comprises one or more additional therapeutic agents.

In some embodiments, the one or more additional therapeutic agents are selected from a corticosteroid, a phosphodiesterase 4 (PDE4) inhibitor, an antibiotic, a Janus kinase (JAK) inhibitor, a Tyk2 inhibitor, a retinoic acid receptor (RAR) compound, an aryl hydrocarbon receptor (AhR) inhibitor, Vitamin D analogue, and a Calcineurin inhibitor.

### Permeation Enhancer

Suitably, the topical composition comprises at least one permeation enhancer. In some embodiments, the topical composition comprises at least two permeation enhancers. Suitably, the topical composition comprises one, two, or three permeation enhancers. More suitably, the topical composition comprises two permeation enhancers. More suitably, the topical composition comprises three permeation enhancers.

A permeation enhancer is an excipient that promotes the absorption of the drug across the stratum corneum of the skin, in this instance Compound A, or a pharmaceutically acceptable salt thereof, temporarily or transiently. Permeation enhancers may operate through various mechanisms to enhance skin permeability. For example, some enhancers, e.g., alcohols such as ethanol, may simply solubilise and extract the lipid component of the stratum corneum to facilitate permeation, whereas fatty acids (e.g., oleic acid) are able to induce lipid fluidization as well as partitioning within the membrane.

Suitable permeation enhancers include di-isopropyl adipate, dimethyl isosorbide (DMI), ethanol, a fatty alcohol (e.g., oleyl alcohol or octyldodecanol), isopropyl myristate (IPM), propylene glycol, glycerine, diethylene glycol monoethyl ether (e.g., marketed as Transcutol® P or Transcutol® HP), a mixture of triglyerides (e.g., a mixture of medium chain triglycerides), caprylic acid, caproic acid, caprylic/capric triglycerides, octanoic acid, decanoic acid, myristic acid, oleic acid, isopropyl alcohol, isostearic acid, hexylene glycol, butylene glycol, diethylsebacate, lecithin, N-methyl pyrrolidone, dimethyl sulfoxide (DMSO), and isopropyl palmitate, or any combination thereof.

A glyceride is an ester derived from glycerol and fatty acids. Glycerol has three hydroxyl functional groups, which can be esterified with one, two, or three fatty acids to form a mono-, di-, or tri-glyceride respectively. A medium chain triglyceride therefore is derived from glycerol which has been esterified with three fatty acids wherein the fatty acids have aliphatic tails of 6-12 carbons. Suitable, medium chain tri-glycerides have a HLB in the range of 12 to 18, suitably about 15.

A suitable permeation enhancer provides skin permeation but does not cause significant systemic absorption. In some embodiments, the permeation enhancer provides skin permeation and causes minimal systemic absorption.

The Applicants have found that certain permeation enhancers can fulfil a dual role by enhancing both permeation and solubilisation of Compound A, or a pharmaceutically acceptable salt thereof, in the composition. Such permeation enhancers serve as a co-solvent for Compound A or a pharmaceutically acceptable salt thereof. Accordingly, in some embodiments, Compound A, or a pharmaceutically acceptable salt thereof, is soluble in at least one permeation enhancer present within the composition.

In some embodiments, Compound A, or a pharmaceutically acceptable salt thereof, has a solubility in the permeation enhancer of at least 2 mg/g when measured at 20±2° C., such as at least 4 mg/g.

In some embodiments, the permeation enhancer is a hydrophilic permeation enhancer. An example of a hydrophilic permeation enhancer is diethylene glycol monoethyl ether.

In some other embodiments, the permeation enhancer is a lipophilic permeation enhancer. Examples of lipophilic permeation enhancers include, but are not limited to, oleyl alcohol and octyldodecanol.

In some embodiments, the topical composition comprises both a hydrophilic permeation enhancer and a lipophilic permeation enhancer.

In some embodiments, the composition comprises one or more permeation enhancers selected from diethylene glycol monoethyl ether, a fatty acid alcohol, a glycol, an aprotic solvent, or any combination thereof.

In some embodiments, the permeation enhancer is a fatty acid alcohol. Examples of fatty acid alcohols include, but are not limited to, lauryl alcohol, stearyl alcohol, oleyl alcohol, and octyldodecanol.

In some embodiments, the fatty acid alcohol is oleyl alcohol, octyldodecanol or a combination thereof.

In some embodiments, the permeation enhancer is a glycol. Examples of glycols include but are not limited to ethylene glycol, diethylene glycol, propylene glycol (e.g., conveniently a super-refined grade), ethylene glycol monobutyl ether, ethylene glycol monomethyl ether, hexylene glycol, butylene glycol, polyethylene glycol, or a combination thereof. In some embodiments, the glycol is propylene glycol (conveniently a super-refined grade), hexylene glycol, butylene glycol, polyethylene glycol, or a combination thereof.

In some embodiments, the aprotic solvent is dimethyl sulfoxide, N-methyl pyrrolidone, or a combination thereof.

In some embodiments, the permeation enhancer is diethylene glycol monoethyl ether.

In some embodiments the composition comprises at least two permeation enhancers and one of these permeation enhancers is diethylene glycol monoethyl ether.

In some embodiments the composition comprises a first permeation enhancer that is diethylene glycol monoethyl ether, a second permeation enhancer that is a fatty acid alcohol and optionally a third permeation enhancer that is also a fatty acid alcohol. Conveniently, the second permeation enhancer is oleyl alcohol and the optional third permeation enhancer is octyldodecanol.

In some embodiments, the permeation enhancer(s) are present in an amount of from about 3 to about 40% w/w of the total composition, such as from about 3 to about 30% w/w, from about 3 to about 20% w/w, from about 3 to about 15% w/w, from about 3 to about 10% w/w, or from about 3 to about 8% w/w. In some embodiments, the permeation enhancer(s) are present in an amount of about 3, about 5 or about 11% w/w of the total composition.

In some embodiments, the permeation enhancer comprises a mixture of diethylene glycol monoethyl ether and at least one fatty acid alcohol.

In some embodiments, the permeation enhancer comprises a mixture of diethylene glycol monoethyl ether and oleyl alcohol. In some embodiments, the permeation enhancer comprises a mixture of diethylene glycol monoethyl ether, oleyl alcohol and octyldodecanol.

In some embodiments, the diethylene glycol monoethyl ether is present in an amount of from about 3 to about 40% w/w of the total composition, such as from about 3 to about 30% w/w, from about 3 to about 20% w/w, from about 3 to about 10% w/w, from about 3 to about 8% w/w. In some embodiments, the diethylene glycol monoethyl ether is present in an amount of about 5% w/w of the total composition. In some embodiments, the diethylene glycol monoethyl ether is present at an amount of no more than 5% w/w of the total composition. The Applicants have surprisingly found that low levels of diethylene glycol monoethyl ether in the topical pharmaceutical composition can help solubilise the compound and can result in the desired permeation and retention of Compound A, or a pharmaceutically acceptable salt thereof, upon application of the composition.

In some embodiments, the diethylene glycol monoethyl ether used to prepare the topical pharmaceutical composition has a purity of at least 99.0%, at least 99.7%, at least 99.9%, at least 99.90%, or at least 99.95%. In some embodiments, the residual solvents present in the diethylene glycol monoethyl ether used to prepare the topical pharmaceutical composition are less than 0.3%, less than 0.1%, or less than 0.05%. In some embodiments, there is no more than 50 ppm of 2-methoxyethanol in the diethylene glycol monoethyl ether used to prepare the topical pharmaceutical composition. In some embodiments, there is no more than 160 ppm of 2-ethoxyethanol in the diethylene glycol monoethyl ether used to prepare the topical pharmaceutical composition. In some embodiments, there is no more than 625 ppm of ethylene glycol in the diethylene glycol monoethyl ether used to prepare the topical pharmaceutical composition. In some embodiments, there is no more than 150 ppm diethylene glycol in the diethylene glycol monoethyl ether used to prepare the topical pharmaceutical composition. Suitable grades of diethylene glycol monoethyl ether include those sold under the tradename, Transcutol® P and Transcutol® HP. In some embodiments the diethylene glycol monoethyl ether is Transcutol® HP.

In some embodiments, the composition comprises oleyl alcohol present in an amount of from about 1 to about 25% w/w of the total composition, such as from about 1 to about 15% w/w, about 1 to about 10% w/w, about 1 to about 5% w/w, or about 2 to about 4% w/w. In some embodiments, the diethylene glycol monoethyl ether is present in an amount of about 3% w/w of the total composition.

In some embodiments, the composition comprises diethylene glycol monoethyl ether and oleyl alcohol. In some embodiments, the weight ratio of the diethylene glycol monoethyl ether to oleyl alcohol is from about 1:2 to about 6:1, conveniently from about 1:1 to about 4:1, more conveniently from about 1:1 to about 2:1. Conveniently, the weight ratio of the diethylene glycol monoethyl ether to oleyl alcohol is about 1.7:1.

In some embodiments, the composition comprises octyldodecanol present at in amount of from about 1 to about 25% w/w of the total composition, such as from about 1 to about 10% w/w, from about 1 to about 5% w/w, or from about 2 to about 4% w/w. In some embodiments, the diethylene glycol monoethyl ether is present in an amount of about 3% w/w of the total composition.

In some embodiments, the weight ratio of the diethylene glycol monoethyl ether to octyldodecanol is between 1:2 and 6:1, conveniently between 1:1 and 4:1, more conveniently between 1:1 and 2:1, such as about 1.7:1.

One aspect relates to a pharmaceutical composition comprising an effective amount of a compound having a structure represented by a formula:

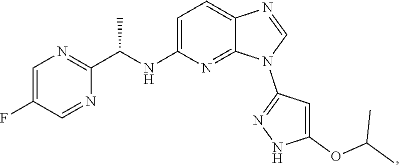 [SMILES: CC(C)Oc1cc(-n2cnc3ccc(N[C@@H](C)c4ncc(F)cn4)nc32)n[nH]1]

a permeation enhancer, and a pharmaceutically acceptable carrier, wherein the composition is free of diisopropyl adipate, isopropyl myristate, light mineral oil, cyclomethicone, and white petrolatum. In a further aspect, the carrier is a topical carrier.

One aspect relates to a pharmaceutical composition comprising an effective amount of a compound having a structure represented by a formula:

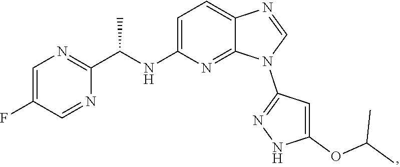 [SMILES: CC(C)Oc1cc(-n2cnc3ccc(N[C@@H](C)c4ncc(F)cn4)nc32)n[nH]1]

a permeation enhancer, an antioxidant, and a pharmaceutically acceptable carrier. In a further aspect, the carrier is a topical carrier. In a further aspect, the composition is a topical composition. In a further aspect, the composition further comprises a pH-adjusting agent as further described herein

In a further aspect, disclosed are methods of treating and/or preventing an inflammatory disorder in a subject in need thereof, the method comprising topically administering to the subject's skin an effective amount of a disclosed composition. In a further aspect, the inflammatory disorder is psoriasis.

### Antioxidant

In some embodiments, the topical pharmaceutical composition further comprises an antioxidant.

As would be readily understood by one of ordinary skill in the art, an antioxidant is a substance that prevents or slows damage to cells caused by free radicals. An antioxidant can also be useful in increasing shelf life of the resultant product.

Examples of suitable antioxidants include, but are not limited to, N-acetyl cysteine, ascorbic acid, glutathione, monothioglycerol, D-α-tocopherol polyethylene glycol succinate (Vitamin E TPGS, e.g., TPGS-1000), sodium metabisulfite, sodium sulfite, sodium thiosulfate, butylated hydroxytoluene (BHT), butylated hydroxyanisole (BHA), vitamin E (e.g., α-tocopherol), vitamin E acetate (e.g., α-tocopherol acetate), tert-butylhydroquinone (TBHQ), propyl gallate, squalene, gallic acid and ascorbyl palmitate.

In some embodiments, the antioxidant is an oil-soluble antioxidant.

Examples of oil-soluble antioxidants include butylated hydroxytoluene (BHT), butylated hydroxyanisole (BHA), propylgallate, squalene, D-alpha tocopherol, or a combination thereof.

In some embodiments, the oil-soluble antioxidant is selected from butylated hydroxytoluene (BHT), butylated hydroxyanisole (BHA), or a combination thereof.

In some embodiments, the oil-soluble antioxidant is butylated hydroxytoluene (BHT).

In some embodiments the antioxidant is butylated hydroxyanisole (BHA). In some embodiments the antioxidant is butylated hydroxytoluene (BHT) and butylated hydroxyanisole (BHA).

Conveniently, the antioxidant is butylated hydroxytoluene (BHT).

In some embodiments, the antioxidant is present in the composition at an amount of from about 0.001% to about 5.0% w/w of the total composition, such as from about 0.001% to about 3.0% w/w, about 0.01% to about 1.0% w/w, about 0.01 to about 0.3% w/w, or about 0.05% to about 0.15% w/w. In some embodiments, the antioxidant is present in the composition at about 0.10% w/w of the total composition.

In some embodiments, the oil-soluble antioxidant is butylated hydroxytoluene (BHT), butylated hydroxyanisole (BHA), or a combination thereof, and the antioxidant is present in the composition at 0.01% to 1.0% w/w of the total composition.

In some embodiments when the compositions comprise diethylene glycol monoethyl ether, the antioxidant is an oil-soluble antioxidant. In some embodiments when the compositions comprise diethylene glycol monoethyl ether, the antioxidant is butylated hydroxytoluene (BHT).

pH-Adjusting Agent

In some embodiments, the topical pharmaceutical composition further comprises a pH adjusting agent. A pH adjusting agent is an excipient which adjusts the pH of the topical pharmaceutical composition to a pH which is well tolerated by the patient upon application of the topical pharmaceutical composition, for example, the topical pharmaceutical composition has a pH which does not cause skin irritation upon application. Suitably, the pH at 25±1° C. of the topical pharmaceutical composition comprising a pH adjusting agent is between about 3.5 and about 7.0, such as between about 4.0 and about 7.0, such as between about 4.5 and about 6.5, such as between about 5.0 and about 6.5, such as about 5.5. In some embodiments, the pH-adjusting agent is a lipophilic pH-adjusting agent.

The Applicants have found that when a pharmaceutically acceptable salt of Compound A is used, for example a mesylate salt, the pH of the topical pharmaceutical composition can drop to about pH 2, which consequently results in skin irritation upon application and also instability of the composition itself. The Applicants investigated a number of different buffering systems to find a system that adjusted and subsequently maintained the pH of the topical pharmaceutical composition to a more suitable value that would be compatible with the skin, approximately pH 5.5. Surprisingly, only certain buffering systems tested provided the preferred desired pH properties.

Furthermore, Applicants also surprisingly found that certain pH adjusting agents were not only able to adjust pH values but were also able to actually improve the stability of certain compositions, e.g. cream compositions, and/or aid solubilisation. For example, without being bound to any theory, it is believed that organic bases with hydroxyl groups may aid in the solubilization of Compound A and due to a high level of solubility with an aqueous phase, may also prevent phase separation for an emulsion-based composition such as a cream.

In some embodiments, the topical pharmaceutical composition comprises a pharmaceutically acceptable salt of Compound A and a pH adjusting agent. In some embodiments, the topical pharmaceutical composition comprises a mesylate salt of Compound A and a pH adjusting agent.

In some embodiments, the pH-adjusting agent is selected such that it can adjust the pH of the composition and also stabilise the composition, e.g. reduce prevent phase separation of an emulsion based cream composition.

In some embodiments, the pH-adjusting agent is a weak organic base. A weak organic base is an organic base with a pKₐ between 8 and 12.

In some embodiments, the weak organic base is an amine. In some embodiments, the weak organic base is a secondary amine or a tertiary amine. In some embodiments, the weak organic base is a tertiary amine.

In some embodiments, the weak organic base is an amine comprising a hydroxy group. In some embodiments, the weak organic base is a secondary amine comprising a hydroxy group or a tertiary amine comprising a hydroxy group. In some embodiments, the weak organic base is a tertiary amine comprising a hydroxy group.

In some embodiments, the weak organic base is a neutral weak organic base at pH 7, i.e. not ionised at pH values in the range of 7 to 11.

In some embodiments, the weak organic base is selected from triethanolamine, tromethamine (Tris; tris(hydroxymethyl)aminomethane), di-isopropyl amine or trometamol, or a combination thereof.

In some embodiments, the weak organic base is triethanolamine.

In some embodiments, diluted solutions of triethanolamine provide benefits in respect to stability and pH of the disclosed compositions.

In some embodiments, the topical pharmaceutical composition comprises a pharmaceutically acceptable salt of Compound A and triethanolamine. In some embodiments, the topical pharmaceutical composition comprises a mesylate salt of Compound A and triethanolamine.

In some embodiments, the molar ratio of the pH-adjusting agent to Compound A is between about 2:1 and about 1:4, conveniently between about 1:1 and about 1:3. In some embodiments, the molar ratio of the pH-adjusting agent to Compound A is about 1:1. In some embodiments, the molar ratio of the pH-adjusting agent to Compound A is about 1:2. In some embodiments, the molar ratio of the pH-adjusting agent to Compound A is about 1:3.

In some embodiments, certain buffers, e.g., phosphate and citrate buffers, can sometimes have a detrimental impact on the stability of certain compositions, e.g. can cause discolouration and phase separation for cream compositions. Certain buffers are also unable to act as effective pH adjusting agents in certain compositions. Accordingly, in some embodiments, the topical pharmaceutical composition does not contain a phosphate and/or citrate buffer.

### Additional Excipients

The composition can include additional excipients, such as one or more counter-irritants, emollients, emulsifiers, solvents, preservatives, humectants and chelating agents or any combinations thereof.

In some embodiments, the topical pharmaceutical composition is a cream and further comprises a counter-irritant selected from menthol, methyl salicylate, camphor, cyclomethicone, dimethicone, dimethicone polyol, cyclomethicone copolyol and cyclopentasiloxanes or a combination thereof. It has been found that the presence of a counter-irritant can be used to balance HLB but has also been particularly beneficial in regards to tolerability for a composition upon continued use.

In some embodiments, the topical pharmaceutical composition comprises between 0.1 and 10% w/w of the counter-irritant.

In some embodiments, when the counter-irritant is cyclomethicone, conveniently present at 1 to 5% w/w of the total composition. Suitably, when the counter-irritant is cyclomethicone, it is present at about 3% w/w of the total composition.

In some embodiments, the composition is a cream and further comprises one or more emollients, emulsifiers, surfactants, solvents, preservatives, humectants and chelating agents or any combinations thereof.

In some embodiments, an emollient is present. In certain embodiments, the emollient comprises cetyl alcohol, a mixture of triglyerides (e.g., a mixture of medium chain triglycerides), paraffin wax, PPG-15 stearyl ether, PPG-11 stearyl ether, stearyl alcohol, white petrolatum, caprylic acid, caproic acid, caprylic/capric triglycerides, octanoic acid, decanoic acid, myristic acid, oleic acid, palmitic acid, isopropyl myristate, stearic acid, linoleic acid, cetyl alcohol, cetostearyl alcohol, glyceryl stearate, Glyceryl Monostearate Type II, cetyl ester wax, bees wax, white wax, cholesterol, paraffin wax, light mineral oil, heavy mineral oil, microcrystalline wax, ozokerite wax, carnuba wax, white bees wax, or a combination thereof in amounts in the range of 0.1 to 15% by weight.

In some embodiments, the emollient comprises a mixture of triglyerides (e.g., a mixture of medium chain triglycerides) and stearyl alcohol. In some embodiments, the emollient comprises cetyl alcohol, a mixture of triglyerides (e.g., a mixture of medium chain triglycerides), stearyl alcohol, and white petrolatum. In some embodiments, the emollient comprises cetyl alcohol, stearyl alcohol, and white petrolatum.

In some embodiments, the emollient comprises paraffin wax, PPG-15 stearyl ether, stearyl alcohol, and white petrolatum.

In some embodiments, the composition comprises an emulsifier. In some embodiments, the emulsifier comprises cetyl alcohol, stearyl alcohol, glycerol monostearate (e.g., glycerol monostearate type II), steareth-2, steareth-20, steareth-21, sorbitan monolaurate (e.g., sold as Span® 20), sorbitan monopalmitate (e.g., sold as Span® 40), sorbitan monostearate (e.g., sold as Span® 60), sorbitan monooleate (e.g., sold as Span® 80), sorbitan trioleate (e.g., sold as Span® 85), polyethylene glycol sorbitan monolaureate (e.g., sold as TWEEN® 20), polyethylene glycol sorbitan monopalmitate (e.g., sold as TWEEN® 40), polyethylene glycol sorbitan monostearate (e.g., sold as TWEEN® 60), polyethylene glycol sorbitan monooleate (e.g., sold as TWEEN® 80), polyoxyl 20 cetylether, polyoxyl 2 cetostearyl ether (e.g., ceteareth-2), polyoxyl 20 cetostearyl ether (e.g., ceteareth-20), polyoxyl 40 stearate, polysorbate (e.g., polysorbate 80), PEG-100 stearate, polyethylene glycol hexadecyl ether (e.g., sold as Cetomacrogol™ 1000), Sepineo™ P600, Tefose® 63, sodium lauryl sulfate, docusate sodium, glyceryl stearate, Glyceryl Monostearate Type II, and PEG-100 stearate, glyceryl oleate, PEG-40 hydrogenated castor oil, cetostearyl alcohol, polyvinyl alcohol, Poloxamer 188, sodium cetostearyl sulfate, or any combination thereof (e.g., at a concentration in the range of about 0.1% w/w to about 10% w/w based on the total weight of the composition).

In some embodiments, the emulsifier comprises stearyl alcohol, steareth-2, steareth-20, and PEG-100 stearate. In some embodiments, the emulsifier comprises cetyl alcohol, stearyl alcohol, glycerol monostearate (e.g., glycerol monostearate type II), and polyoxyl 40 stearate. In some embodiments, the emulsifier comprises cetyl alcohol, stearyl alcohol, glycerol monostearate (e.g., glycerol monostearate type II), sorbitan monooleate (e.g., sold as Span® 80), polysorbate (e.g., polysorbate 80), and Sepineo™ P600. In some embodiments, the emulsifier comprises stearyl alcohol, glycerol monostearate (e.g., glycerol monostearate type II), and polyethylene glycol hexadecyl ether (e.g., sold as Cetomacrogol™ 1000).

In some embodiments, the composition comprises a solvent. In some embodiments, the solvent comprises water. In some embodiments, the solvent is present in the composition at between 20 to 70% w/w of the total composition, such as between 30 and 60% w/w, such as between 40 and 50% w/w, such as between 45 and 48% w/w, such as about 47% w/w.

In some embodiments, the composition comprises a preservative. In some embodiments, the preservative comprises sorbic acid, a paraben (e.g., methyl paraben or propyl paraben), sodium methyl paraben, sodium propyl paraben, sorbitol solution, thimersal, quaternary ammonium salts (NH4+ salts), benzalkonium chloride, potassium permanganate, cealkonium chloride, cetyl pyridinium chloride, cetrimide, quaternium-15, sodium benzoate, imidurea, diazolidinyl urea, chlorhexidine gluconoate, urea, DMDM hydantoin, isochlorthiozolines, benzoic acid, benzyl alcohol, phenoxyethanol, or any combination thereof (e.g., at a concentration in the range of about 0.1% w/w to about 10% w/w).

In some embodiments, the preservative comprises sorbic acid, methyl paraben, and propyl paraben.

In some embodiments, the composition comprises a humectant. In certain embodiments, the humectant comprises glycerin, sorbitol, propylene glycol, butylene glycol, hexylene glycol, (polyethylene glycol)n where n=200, 300, 400, 550, 600, or 1000, water, or a combination thereof (e.g., at a concentration in the range of about 0.1% w/w to about 50% w/w).

In some embodiments, the composition comprises a chelating agent. In certain embodiments, the chelating agent comprises ethylenediaminetetraacetic acid (EDTA), disodium edetate, calcium EDTA, or a combination thereof (e.g., at a concentration in the range of about 0.1% w/w to about 10% w/w).

### Stability

In some embodiments, the topical pharmaceutical composition is stable.

A suitable topical pharmaceutical composition is stable when stored at 25° C./60% RH and/or 40° C./75% RH for at least 1 month, such as at least 2 months, 3 months, 4 months, 6 months, 9 months, 12 months, 18 months, 24 months or 36 months.

In some embodiments, the topical pharmaceutical composition is stable when stored at 25° C. and 60% RH for about 1 month.

In some embodiments, the topical pharmaceutical composition is chemically stable. In some embodiments, Compound A or a pharmaceutically acceptable salt thereof is stable within the topical pharmaceutical composition and the topical pharmaceutical composition provides less than 5% (conveniently less than 3%) chemical degradation of the Compound A or pharmaceutically acceptable salt thereof by HPLC when stored at 25° C./60% RH and/or 40° C./75% RH for at least 1 month, such as at least 2 months, 3 months, 4 months, 6 months, 9 months, 12 months, 18 months, 24 months or 36 months. In some embodiments, Compound A, or a pharmaceutically acceptable salt thereof, is stable within the topical pharmaceutical composition and the topical pharmaceutical composition provides less than 5% (conveniently less than 3%) chemical degradation of the Compound A or pharmaceutically acceptable salt thereof by HPLC when stored at 25° C./60% RH and/or 40° C./75% RH for at least about 1 month.

In some embodiments, the topical pharmaceutical composition is physically stable. In some embodiments, the topical pharmaceutical composition is physically stable when stored at 25° C./60% RH and/or 40° C./75% RH for at least 1 month, such as at least 2 months, 3 months, 4 months, 6 months, 9 months, 12 months, 18 months, 24 months or 36 months. Physically stable includes, but is not limited to, one or more of the following properties: (a) stable to phase separation, (b) stable to salt dissociation, (c) stable to segregation, (d) has no colour change, (e) no change or shift in pH of the composition, and (f) to the extent that Compound A is dissolved in the topical pharmaceutical composition, it remains in the dissolved state. The skilled person would be able to determine whether the topical pharmaceutical composition was physically stable using different analytical techniques. For example, phase separation and assessment of the dissolved state of Compound A, or pharmaceutically acceptable salt thereof can be determined by eye and/or microscopy. Segregation can be determined by assaying portions of the topical pharmaceutical composition to ascertain if Compound A, or a pharmaceutically acceptable salt thereof, is uniformly distributed.

In some embodiments, the topical pharmaceutical composition is a cream and is physically stable to phase separation when stored at 25° C./60% RH and/or 40° C./75% RH for at least 1 month, such as 2 months, 3 months, 4 months, 6 months, 9 months, 12 months, 18 months, 24 months, or 36 months.

In some embodiments, the topical pharmaceutical composition is a cream and is physically stable to phase separation when stored at 25° C./60% RH and/or 40° C./75% RH for about 1 month.

In some embodiments, no change in the colour of the topical pharmaceutical composition is observed when stored at 25° C./60% RH and/or 40° C./75% RH for at least 1 month, such as 2 months, 3 months, 4 months 6 months, 9 months, 12 months, 18 months, 24 months, or 36 months.

In some embodiments, there is no change in colour of the topical pharmaceutical composition is observed when stored at 25° C./60% RH and/or 40° C./75% RH for about 1 month.

In some embodiments, the pH of the topical pharmaceutical composition is in the range of from about 3.5 to about 7.0, conveniently, from about 4.5 to about 6.5 when stored at 25° C./60% RH and/or 40° C./75% RH for at least 1 month, such as 2 months, 3 months, 4 months 6 months, 9 months, 12 months, 18 months, 24 months or 36 months.

In some embodiments, the pH of the topical pharmaceutical composition is in the range of from about 3.5 to about 7.0, conveniently, from about 4.5 to about 6.5 when stored at 25° C./60% RH and/or 40° C./75% RH for about 1 month.

### Therapeutic Characteristics

In some embodiments, the topical pharmaceutical composition is well tolerated with no dermal irritation or only minimal erythema when applied to the skin.

In some embodiments, when the topical pharmaceutical composition is subject to an in-vitro permeation test, as outlined in Example 9, the topical composition provides a flux of less than 5 ng/cm²·h, such as less than 2 ng/cm²·h, 1.5 ng/cm²·h, or 1.0 ng/cm²·h.

In some embodiments, the area under the plasma concentration-time curve from time zero up to 24 hours post-dose (AUC₀₋₂₄) after the composition is administered topically to the skin of the subject in a single application is less than approximately 100 ·hr/mL.

### Particular Compositions

In some embodiments, the topical pharmaceutical composition comprises diethylene glycol monoethyl ether, oleyl alcohol and an oil-soluble antioxidant.

In some embodiments, the topical pharmaceutical composition comprises diethylene glycol monoethyl ether, oleyl alcohol and an oil-soluble antioxidant, wherein the oil-soluble antioxidant comprises butylated hydroxytoluene (BHT), butylated hydroxyanisole (BHA), or a combination thereof and the antioxidant is present in the composition at 0.01% to 1.0% w/w of the total composition.

In some embodiments, the topical pharmaceutical composition is a cream and comprises a mesylate salt of Compound A, diethylene glycol monoethyl ether, an oil-soluble antioxidant and a pH adjusting agent, wherein the pH adjusting agent is a tertiary amine.

In some embodiments, the topical pharmaceutical composition comprises:
a) Compound A or a pharmaceutically acceptable salt thereof:
b) at least one permeation enhancer; and
c) one or more pharmaceutically acceptable excipients;
wherein the pH of the topical pharmaceutical composition at 25±1° C. is in the range of 4.5 to 7; and
wherein the topical pharmaceutical composition provides less than 5% (conveniently less than 3%) chemical degradation of the Compound A or pharmaceutically acceptable salt thereof by HPLC when stored at 25° C./60% RH and/or 40° C./75% RH for at least about 1 month (conveniently at least about 3 months, more conveniently at least about 6 months, at least about 9 months, at least about 12 months, at least about 18 months, at least about 24 months, or at least 36 months).

In some embodiments, the topical pharmaceutical composition comprises:
a) Compound A or a pharmaceutically acceptable salt thereof:
b) at least one permeation enhancer;
c) an antioxidant; and
d) one or more pharmaceutically acceptable excipients.

In some embodiments, the topical pharmaceutical composition comprises:
a) a mesylate salt of Compound A:
b) at least one permeation enhancer;
c) an antioxidant;
d) a pH adjusting agent; and
e) one or more pharmaceutically acceptable excipients.

In some embodiments, the topical pharmaceutical composition comprises:
a) a mesylate salt of Compound A:
b) at least one permeation enhancer;
c) an antioxidant;
d) a pH adjusting agent; and
e) one or more pharmaceutically acceptable excipients;
wherein the pH of the topical pharmaceutical composition at 25±1° C. is in the range of 4.5 and 7.

In some embodiments, the topical pharmaceutical composition comprises:
a) a mesylate salt of Compound A:
b) at least one permeation enhancer;
c) an antioxidant;
d) a pH adjusting agent; and
e) one or more pharmaceutically acceptable excipients;
wherein the pH of the topical pharmaceutical composition at 25±1° C. is in the range of 4.5 and 7; and
wherein the topical composition is physically stable when stored at 25° C./60% RH and/or 40° C./75% RH for at least 1 month.

In some embodiments, the topical pharmaceutical composition comprises:
a) between 0.005% w/w and 5% w/w of Compound A or a pharmaceutically acceptable salt thereof;
b) 3 to 40% w/w of the permeation enhancer; and
c) one or more pharmaceutically acceptable excipients;
wherein weights are based on the total weight of the composition.

In some embodiments, the topical pharmaceutical composition comprises:
a) between 0.005% w/w and 5% w/w of Compound A or a pharmaceutically acceptable salt thereof;
b) 3 to 40% w/w of the permeation enhancer; and
c) 0.001% to 5.0% w/w of an antioxidant;
wherein weights are based on the total weight of the composition.

In some embodiments, the topical pharmaceutical composition comprises:
a) between 0.005% w/w and 5% w/w of the mesylate salt of Compound A:
b) 3 to 40% w/w of the permeation enhancer;
c) 0.001% to 5.0% w/w of an antioxidant; and
d) a pH adjusting agent;
wherein weights are based on the total weight of the composition.

In some embodiments, the topical pharmaceutical composition comprises:
a) the mesylate salt of Compound A:
b) diethylene glycol monoethyl ether;
c) butylated hydroxy toluene; and
d) triethanolamine.

In some embodiments, the topical pharmaceutical composition comprises:
a) between 0.005% w/w and 0.15% w/w of the mesylate salt of Compound A:
b) 3 to 8% w/w of diethylene glycol monoethyl ether;
c) 0.05% to 0.15% w/w butylated hydroxy toluene; and
d) triethanolamine;
wherein weights are based on the total weight of the composition.

In some embodiments, the topical pharmaceutical composition comprises:
a) between 0.005% w/w and 5% w/w of the mesylate salt of Compound A:
b) 3 to 40% w/w of diethylene glycol monoethyl ether;
c) 0.001% to 5.0% w/w butylated hydroxy toluene; and
d) triethanolamine;
wherein weights are based on the total weight of the composition.

In some embodiments, the topical pharmaceutical composition comprises:
a) about 0.03% w/w of the mesylate salt of Compound A:
b) about 5% w/w of diethylene glycol monoethyl ether;
c) about 0.10% w/w butylated hydroxy toluene; and
d) triethanolamine;
wherein weights are based on the total weight of the composition.

In some embodiments, the topical pharmaceutical composition comprises:
a) about 0.01% w/w of the mesylate salt of Compound A:
b) about 5% w/w of diethylene glycol monoethyl ether;
c) about 0.10% w/w butylated hydroxy toluene; and
d) triethanolamine;
wherein weights are based on the total weight of the composition.

In some embodiments, the topical pharmaceutical composition comprises:
a) about 0.10% w/w (or 0.20% w/w) of the mesylate salt of Compound A:
b) about 5% w/w of diethylene glycol monoethyl ether;
c) about 0.10% w/w butylated hydroxy toluene; and
d) triethanolamine;
wherein weights are based on the total weight of the composition.

In some embodiments, the topical pharmaceutical composition comprises:
a) about 0.30% w/w of the mesylate salt of Compound A:
b) about 5% w/w of diethylene glycol monoethyl ether;
c) about 0.10% w/w butylated hydroxy toluene; and
d) triethanolamine;
wherein weights are based on the total weight of the composition.

In some embodiments, the topical pharmaceutical composition comprises:
a) about 1% w/w of the mesylate salt of Compound A:
b) about 10% w/w of diethylene glycol monoethyl ether;
c) about 0.10% w/w butylated hydroxy toluene; and
d) triethanolamine;
wherein weights are based on the total weight of the composition.

In some embodiments, the topical pharmaceutical composition comprises:
a) about 3% w/w of the mesylate salt of Compound A:
b) about 10% w/w of diethylene glycol monoethyl ether;
c) about 0.10% w/w butylated hydroxy toluene; and
d) triethanolamine;
wherein weights are based on the total weight of the composition.

### Processes of Preparation

One aspect relates to a process for a preparing a topical composition as herein defined, wherein the process comprises the steps of:
a) providing Compound A or a pharmaceutically acceptable salt thereof in an aqueous phase, wherein the aqueous phase optionally also comprises at least one hydrophilic emulsifier and/or at least one preservative and/or at least one chelating agent to provide a homogenous aqueous phase;
b) further providing at least one permeation enhancer in the aqueous phase;
c) emulsifying the aqueous phase at an elevated temperature (about 50° C. to about 70° C.) into an oil phase to form an emulsion, then allowing the emulsion to cool to form a topical composition (such as a cream), wherein the oil phase comprises at least one emollient, at least one emulsifier and at least one antioxidant and optionally also comprises at least one preservative, at least one further permeation enhancer and at least one counter-irritant; and
d) optionally adjusting the pH of the composition by incorporating a pH-adjusting agent.

Applicants have surprisingly found that the point at which a permeation enhancer is added to the aqueous phase of step a) may enhance the stability of the final composition. Addition of the permeation enhancer into the aqueous just prior to the emulsification step can reduce physical instability such as liquid syneresis and other destabilisation of the composition over time. Accordingly, in some embodiments, the permeation enhancer (e.g. diethylene glycol monoethyl ether) is incorporated prior to, or just prior to, emulsification, i.e. in step b) in the process described above.

In a further aspect, there is provided a process for preparing a topical composition as herein defined, wherein the process involves the steps of:
a) providing Compound A, or a pharmaceutically acceptable salt thereof in an aqueous phase to provide a solution, wherein the aqueous phase optionally also comprises at least one hydrophilic emulsifier and/or at least one preservative and/or at least one chelating agent;
b) further providing at least one permeation enhancer in the aqueous phase and heating the mixture to a temperature in the range of about 50° C. to about 70° C. (conveniently about 60° C.) with the aid of stirring;
c) emulsifying the aqueous phase into an oil phase at a temperature in the range of about 50° C. to about 70° C. (conveniently about 60° C.) to form an emulsion, then allowing the emulsion to cool to form a topical composition (such as a cream), wherein:
   i. the oil phase comprises at least one emollient, at least one emulsifier and at least one antioxidant and optionally also comprises at least one preservative, at least one permeation enhancer and at least one counter-irritant;
   ii. the oil phase is formed by incorporating and mixing the components at a temperature in the range of about 60° C. to about 80° C. (conveniently about 70° C.) with the aid of stirring; and
   iii. the emulsification is provided by homogenisation (optionally under vacuum); and
d) optionally adjusting the pH of the composition by incorporating a pH-adjusting agent.

In a further aspect, there is provided a process for preparing a topical composition as herein defined, wherein the process involves the steps of:
a) providing Compound A, or a pharmaceutically acceptable salt thereof in an aqueous phase to provide a clear solution, wherein the aqueous phase also comprises polysorbate 80, methyl paraben and disodium edetate;
b) further providing diethylene glycol monoethyl ether in the aqueous phase;
c) emulsifying the aqueous phase at an elevated temperature into an oil phase to form an emulsion, then allowing the emulsion to cool to form a topical composition (such as a cream), wherein the oil phase comprises cetyl alcohol, stearyl alcohol, white petrolatum, glyceryl monostearate, butylated hydoxy toluene, sorbic acid, propyl paraben, sorbitan monooleate, oleyl alcohol, octyldodecanol and cyclomethicone; and
d) optionally adjusting the pH of the composition by incorporating trolamine to provide a final pH of a final pH of about 4.5 to 6.5.

In a further aspect, there is provided a process for preparing a topical composition as herein defined, wherein the process involves the steps of:
a) providing the mesylate salt of Compound A in an aqueous phase to provide a clear solution, wherein the aqueous phase also comprises polysorbate 80, methyl paraben and disodium edetate;
b) further providing diethylene glycol monoethyl ether in the aqueous phase then heating the mixture to a temperature in the range of about 50° C. to about 70° C. (conveniently about 60° C.) with the aid of stirring;
c) emulsifying the aqueous phase into an oil phase at a temperature in the range of about 50° C. to about 70° C. (conveniently about 60° C.) and allowing the emulsion to cool to form a topical composition (such as a cream), wherein the oil phase comprises cetyl alcohol, stearyl alcohol, white petrolatum, glyceryl monostearate, butylated hydoxy toluene, sorbic acid, propyl paraben, sorbitan monooleate, oleyl alcohol, octyldodecanol and cyclomethicone; and
d) adjusting the pH of the composition by incorporating trolamine to provide a final pH of a final pH of about 4.5 to 6.5.

A further aspect relates to a process for preparing a topical pharmaceutical composition, the process comprising the steps of:
a) providing Compound A or a pharmaceutically acceptable salt thereof in an aqueous phase;
b) providing a further aqueous phase comprising at least one hydrophilic emulsifier (e.g. polysorbate, for example polysorbate 80) and optionally at least one preservative and/or at least one chelating agent to provide a homogenous aqueous phase;
c) further providing at least one permeation enhancer in the aqueous phase a) or b);
d) emulsifying the aqueous phase of a) and b) at an elevated temperature into an oil phase to form an emulsion, then allowing the emulsion to cool to form a topical composition (such as a cream or ointment), wherein the oil phase comprises at least one emollient, at least one emulsifier and at least one antioxidant and optionally also comprises at least one preservative, at least one further permeation enhancer and at least one counter-irritant; and
e) optionally adjusting the pH of the composition by incorporating a pH-adjusting agent.

In a further aspect, there is provided a process for preparing a topical composition as herein defined, wherein the process involves the steps of:
a) providing Compound A or a pharmaceutically acceptable salt thereof in an aqueous phase;
b) providing a further aqueous phase comprising at least one hydrophilic emulsifier (e.g. polysorbate, for example polysorbate 80) and optionally at least one preservative and/or at least one chelating agent to provide a homogenous aqueous phase;
c) further providing at least one permeation enhancer in the aqueous phase a) or b) and heating the mixture to a temperature in the range of about 50° C. to about 70° C. (conveniently about 60° C.) with the aid of stirring;
d) emulsifying the aqueous phase of a) and b) into an oil phase at a temperature in the range of about 50° C. to about 70° C. (conveniently about 60° C.) to form an emulsion, then allowing the emulsion to cool to form a topical composition (such as a cream or ointment):
   i. wherein the oil phase comprises at least one emollient, at least one emulsifier and at least one antioxidant and optionally also comprises at least one preservative, at least one further permeation enhancer and at least one counter-irritant;
   ii. the oil phase is formed by incorporating and mixing the components at a temperature in the range of about 60° C. to about 80° C. (conveniently about 70° C.) with the aid of stirring; and
   iii. the emulsification is provided by homogenisation (optionally under vacuum); and
e) optionally adjusting the pH of the composition by incorporating a pH-adjusting agent.

In a further aspect, there is provided a process for preparing a topical composition as herein defined, wherein the process involves the steps of:
a) providing Compound A or a pharmaceutically acceptable salt thereof in an aqueous phase;
b) providing a further aqueous phase comprising polysorbate 80, methyl paraben and disodium edetate;
c) further providing diethylene glycol monoethyl ether in the aqueous phase a) or b);
d) emulsifying the aqueous phase of a) and b) at an elevated temperature into an oil phase to form an emulsion, then allowing the emulsion to cool to form a topical composition (such as a cream or ointment), wherein the oil phase comprises cetyl alcohol, stearyl alcohol, white petrolatum, glyceryl monostearate, butylated hydoxy toluene, sorbic acid, propyl paraben, sorbitan monooleate, oleyl alcohol, octyldodecanol and cyclomethicone; and
e) optionally adjusting the pH of the composition by incorporating trolamine to provide a final pH of a final pH of about 4.5 to 6.5.

In a further aspect, there is provided a process for preparing a topical composition as herein defined, wherein the process involves the steps of:
a) providing the mesylate salt of Compound A in an aqueous phase;
b) providing a further aqueous phase comprising polysorbate 80, methyl paraben and disodium edetate;
c) further providing diethylene glycol monoethyl ether in the aqueous phase a) or b) then heating the mixture to a temperature in the range of about 50° C. to about 70° C. (conveniently about 60° C.) with the aid of stirring;
d) emulsifying the aqueous phase of a) and b) into an oil phase at a temperature in the range of about 50° C. to about 70° C. (conveniently about 60° C.) and allowing the emulsion to cool to form a topical composition (such as a cream), wherein the oil phase comprises cetyl alcohol, stearyl alcohol, white petrolatum, glyceryl monostearate, butylated hydoxy toluene, sorbic acid, propyl paraben, sorbitan monooleate, oleyl alcohol, octyldodecanol and cyclomethicone;
e) adjusting the pH of the composition by incorporating trolamine to provide a final pH of a final pH of about 4.5 to 6.5.

In some embodiments, the process (or parts of the process) for preparing a topical composition as herein defined is performed in an inert atmosphere, for example, a nitrogen atmosphere.

In a further aspect, there is provided a process for preparing a composition as herein defined, wherein the process comprises combining: (a) Compound A or a pharmaceutically acceptable salt thereof; and (b) a permeation enhancer.

In a further aspect, Compound A, or the pharmaceutically acceptable salt thereof, is present in an aqueous phase. In a further aspect, the aqueous phase further comprises one or more selected from a hydrophilic emulsifier, a preservative, and a chelating agent, or a combination thereof. In a further aspect, the aqueous phase further comprises polysorbate 80, methyl paraben, and disodium edetate.

In a further aspect, the permeation enhancer is diethylene glycol monoethyl ether.

In a further aspect, combining is at a temperature of from about 40° C. to about 80° C., about 40° C. to about 70° C., about 50° C. to about 80° C., or about 50° C. to about 70° C. In a further aspect, combining is at a temperature of from about 50° C. to about 70° C. In a further aspect, combining is at a temperature of about 60° C.

In a further aspect, combining is with stirring.

In a further aspect, the method further comprises emulsifying the aqueous phase into an oil phase, thereby forming an emulsion.

In a further aspect, emulsifying is at an elevated temperature. In a further aspect, emulsifying is at a temperature of from about 40° C. to about 80° C., about 40° C. to about 70° C., about 50° C. to about 80° C., about 60° C. to about 80° C., or about 50° C. to about 70° C. In a further aspect, the elevated temperature is from about 50° C. to about 70° C. In a further aspect, emulsifying is at a temperature of about 60° C.

In a further aspect, emulsifying is under N₂ such as, for example, 100% N₂.

In a further aspect, emulsifying is via homogenization such as, for example, homogenization under vacuum.

In a further aspect, the oil phase comprises one or more selected from an emollient, an emulsifier, and an antioxidant, or a combination thereof. In a further aspect, the oil phase further comprises one or more selected from a preservative, a permeation enhancer, and a counterirritant, or a combination thereof. In a further aspect, the oil phase comprises cetyl alcohol, stearyl alcohol, white petrolatum, glyceryl monostearate, butylated hydroxy toluene, sorbic acid, propyl paraben, sorbitan monooleate, olely alcohol, octyldodecanol, and cyclomethicone.

In a further aspect, the method further comprises cooling the emulsion.

In a further aspect, the method further comprises incorporating a pH-adjusting agent.

In a further aspect, the method further comprises adjusting the pH of the composition by incorporating a pH-adjusting agent. For example, the pH of the composition can be adjusted to a final pH of from about 3.5 to about 7.5, about 3.5 to about 7.0, about 3.5 to about 6.5, about 3.5 to about 6.0, about 3.5 to about 5.5, about 4.0 to about 7.5, about 4.5 to about 7.5, about 5.0 to about 7.5, about 4.0 to about 7.0, about 4.5 to about 6.5, or about 5.0 to about 6.0. In a further aspect, the pH of the composition can be adjusted to a final pH of from about 4.5 to about 6.5, such as from about 5.0 to about 6.0.

In another aspect, there is provided a product obtainable by any of the processes herein described.

### Therapeutic Uses

Compositions of this disclosure comprising Compound A possess TRK inhibitory activity and accordingly such compositions are useful in a number of medical diseases and conditions associated with TRK dysregulation. As such, in one aspect of the present disclosure, there is provided a topical pharmaceutical composition according to the disclosure as herein defined for use as a medicament.

Compound A is a highly specific pan-TRK inhibitor with a mechanism of action that is competitive with the adenosine triphosphate (ATP) binding site of TRKA. The compound has a potent half maximal inhibitory concentration (IC₅₀) of 0.2 nM for TRKA, in cells. Compound A also shows in-vitro activity against TRKB and TRKC receptor isoforms at concentrations below 5 nM, in cell-based systems overexpressing wild-type, full-length, human TRK receptors.

The present disclosure further provides a method of treating diseases or conditions mediated alone, or in part, by tropomyosin-related kinases (TRKs), the method comprising administering to a subject a therapeutically effective amount of a topical pharmaceutical composition of the present disclosure. The present disclosure also provides a therapeutically effective amount of a topical pharmaceutical composition of the present disclosure for use in treating diseases or conditions mediated alone, or in part, by tropomyosin-related kinases (TRKs).

The present disclosure further provides a composition for use in a method of treating diseases or conditions mediated alone, or in part, by tropomyosin-related kinases (TRKs), the method comprising administering to a subject a therapeutically effective amount of a topical pharmaceutical composition of the present disclosure. The present disclosure also provides a therapeutically effective amount of a topical pharmaceutical composition of the present disclosure for use in treating diseases or conditions mediated alone, or in part, by tropomyosin-related kinases (TRKs).

In some embodiments, there is provided a method of inhibiting a tropomyosin-related kinase (TRK) in a subject, comprising administering to the subject the topical pharmaceutical composition disclosed herein. In some embodiments, there is provided a therapeutically effective amount of a topical pharmaceutical composition as defined herein for use in inhibiting a tyrosine receptor kinase in a subject.

In some embodiments, there is provided a composition for use in inhibiting a tropomyosin-related kinase (TRK) in a subject, comprising administering to the subject the topical pharmaceutical composition disclosed herein. In some embodiments, there is provided a therapeutically effective amount of a topical pharmaceutical composition as defined herein for use in inhibiting a tyrosine receptor kinase in a subject.

In some embodiments, the disease is associated with elevated expression or activity of a tropomyosin-related kinase (TRK). Dysregulation of TRK may refer to either elevated expression or elevated activity of TRK.

In some embodiments, the administration of the topical pharmaceutical composition results in an inhibition of the tropomyosin-related kinase (TRK).

In some embodiments, the administration of the topical pharmaceutical composition comprising Compound A, or a pharmaceutically acceptable salt thereof results in a reduced activity of the tropomyosin-related kinase (TRK).

In some embodiments, administration of the topical pharmaceutical composition comprising Compound A, or a pharmaceutically acceptable salt thereof reduces the activity of TRKA, TRKB, and/or TRKC. In some embodiments, the composition reduces the activity of TRKA. In some embodiments, the composition reduces the activity of TRKB. In some embodiments, the composition reduces the activity of TRKC.

In some embodiments, the disclosure provides a method for treating an immune-mediated disease comprising administering to a subject in need thereof the topical pharmaceutical composition comprising Compound A or a pharmaceutically acceptable salt thereof, as defined herein.

In some embodiments, the disclosure provides a composition for use in treating an immune-mediated disease comprising administering to a subject in need thereof the topical pharmaceutical composition comprising Compound A or a pharmaceutically acceptable salt thereof, as defined herein.

In some embodiments, the disclosure provides a method for treating a disease associated with an abnormal immune response wherein the method comprises administering to a subject in need thereof the topical pharmaceutical composition comprising Compound A or a pharmaceutically acceptable salt thereof, as defined herein.

In some embodiments, the disclosure provides a composition for use in treating a disease associated with an abnormal immune response comprising administering to a subject in need thereof the topical pharmaceutical composition comprising Compound A or a pharmaceutically acceptable salt thereof, as defined herein.

In some embodiments, the disclosure provides a method for treating an inflammatory disease wherein the method comprises administering to a subject in need thereof the topical pharmaceutical composition comprising Compound A or a pharmaceutically acceptable salt thereof, as defined herein.

In some embodiments, the disclosure provides a composition for use in treating an inflammatory disease comprising administering to a subject in need thereof the topical pharmaceutical composition comprising Compound A or a pharmaceutically acceptable salt thereof, as defined herein.

In some embodiments, the disclosure provides a method for treating an inflammatory skin disorder comprising administering to a subject in need thereof the topical pharmaceutical composition comprising Compound A or a pharmaceutically acceptable salt thereof, as defined herein. In some embodiments, the inflammatory skin condition is psoriasis, actinic keratosis, psoriasis guttata, inverse psoriasis, pustular psoriasis, psoriatic erythroderma, acute febrile neutrophilic dermatosis, eczema, xerotic eczema, dyshidrotic eczema, vesicular palmar eczema, acne vulgaris, atopic dermatitis, contact dermatitis, allergic contact dermatitis, dermatomyositis, exfoliative dermatitis, hand eczema, pompholyx, keloids, rosacea, rosacea due to sarcoidosis, rosacea due to scleroderma, rosacea due to Sweet syndrome, rosacea due to systemic lupus erythematosus, rosacea due to urticaria, rosacea due to herpetic pain, mastocytosis, uticaria, Sweet's disease, neutrophilic hydrodenitis, sterile pustule, drug rash, seborrheic dermatitis, Pityriasis rosea, Kikuchi's disease of the skin, pruritic urticarial papules and plaques of pregnancy, Stevens-Johnson syndrome and toxic epidermal necrolysis, tattoo reaction, Wells syndrome (eosinophilic cellulitis), reactive arthritis (Reiter syndrome), bowel-associated dermatosis-arthritis syndrome, rheumatoid neutrophilic dermatosis, neutrophilic eccrine hidradenitis, neutrophilic skin disease of dorsum of hand, balanitis circumscripta plasmacellularis, balanoposthitis, Behcet's disease, erythema annulare centrifugum, erythema dyschromicum perstans, erythema multiforme, granuloma annulare, dermatitis of hand, Lichen nitidus, Lichen planus, Lichen sclerosus et atrophicus, Lichen simplex chronicus, Lichen spinulosus, nummular dermatitis, pyoderma gangrenosum, sarcoidosis, subcorneal pustular dermatosis, urticaria, transient acantholytic dermatosis, bullous pemphigoid, dermatitis herpetiformis, dermatophytic infections, folliculitis, pediculosis, scabies, oral Lichen planus or aphthous ulcers. The method comprises administering to a subject a therapeutically effective amount of a topical pharmaceutical composition of the present disclosure as herein defined.

In some embodiments, the disclosure provides a composition as defined herein for use in treating an inflammatory skin disorder comprising administering to a subject in need thereof the topical pharmaceutical composition comprising Compound A or a pharmaceutically acceptable salt thereof. In some embodiments, the inflammatory skin disorder is psoriasis, actinic keratosis, psoriasis guttata, inverse psoriasis, pustular psoriasis, psoriatic erythroderma, acute febrile neutrophilic dermatosis, eczema, xerotic eczema, dyshidrotic eczema, vesicular palmar eczema, acne vulgaris, atopic dermatitis, contact dermatitis, allergic contact dermatitis, dermatomyositis, exfoliative dermatitis, hand eczema, pompholyx, keloids, rosacea, rosacea due to sarcoidosis, rosacea due to scleroderma, rosacea due to Sweet syndrome, rosacea due to systemic lupus erythematosus, rosacea due to urticaria, rosacea due to herpetic pain, mastocytosis, uticaria, Sweet's disease, neutrophilic hydrodenitis, sterile pustule, drug rash, seborrheic dermatitis, Pityriasis rosea, Kikuchi's disease of the skin, pruritic urticarial papules and plaques of pregnancy, Stevens-Johnson syndrome and toxic epidermal necrolysis, tattoo reaction, Wells syndrome (eosinophilic cellulitis), reactive arthritis (Reiter syndrome), bowel-associated dermatosis-arthritis syndrome, rheumatoid neutrophilic dermatosis, neutrophilic eccrine hidradenitis, neutrophilic skin disease of dorsum of hand, balanitis circumscripta plasmacellularis, balanoposthitis, Behcet's disease, erythema annulare centrifugum, erythema dyschromicum perstans, erythema multiforme, granuloma annulare, dermatitis of hand, Lichen nitidus, Lichen planus, Lichen sclerosus et atrophicus, Lichen simplex chronicus, Lichen spinulosus, nummular dermatitis, pyoderma gangrenosum, sarcoidosis, subcorneal pustular dermatosis, urticaria, transient acantholytic dermatosis, bullous pemphigoid, dermatitis herpetiformis, dermatophytic infections, folliculitis, pediculosis, scabies, oral Lichen planus or aphthous ulcers.

In some embodiments, the inflammatory skin disorder is psoriasis or atopic dermatitis.

In some embodiments, the inflammatory skin disorder is psoriasis.

In some embodiments, the psoriasis is selected from plaque psoriasis, guttate psoriasis, inverse psoriasis, mild to moderate psoriasis, moderate to severe psoriasis, pustular psoriasis and erythrodermic psoriasis.

In some embodiments, the disclosure provides a method for treating dermatoses comprising administering to a subject in need thereof the topical pharmaceutical composition as defined herein. In some embodiments the dermatoses is associated with pruritus.

In some embodiments, the disclosure provides a composition as defined herein for use in treating dermatoses comprising administering to a subject in need thereof the topical pharmaceutical composition. In some embodiments the dermatoses is associated with pruritus.

### Doses

Effective doses of the topical composition of this disclosure will vary, as recognized by those skilled in the art, depending on the diseases treated, the severity of the disease, the sex, age and general health condition of the subject, excipient usage, the possibility of co-usage with other therapeutic treatments such as use of other agents and the judgment of the treating physician. The administration of Compound A, or a pharmaceutically acceptable salt thereof, can continue for as long as necessary to treat the disorder, e.g., for one week, two weeks, one month, two months, three months, four months, six months, one year, two years, five years, ten years, or longer.

A therapeutically effective dose refers to an amount of a composition comprising Compound A, or a pharmaceutically acceptable salt thereof, as described herein, effective to achieve a particular biological or therapeutic result such as, but not limited to, biological or therapeutic results disclosed, described, or exemplified herein. A daily dose in the range of 0.001-15 mg per kg body weight may be employed.

### Combinations

Topical pharmaceutical compositions of the present disclosure may be administered alone as a sole therapy or can be administered in addition with one or more other therapeutic agents. Such conjoint treatment may be achieved by way of the simultaneous, sequential or separate administration of the individual components of the treatment.

The particular choice of other therapeutic agent will depend upon the diagnosis of the attending physicians and their judgment of the condition of the individual and the appropriate treatment protocol. In certain embodiments, Compound A, or a pharmaceutically acceptable salt thereof, is administered in combination with a second therapeutic agent and conveniently the second therapeutic agent is an agent useful in the treatment of conditions mediated alone, or in part, by tropomyosin-related kinases (TRKs). Other second therapeutic agents include, but are not limited to, topical corticosteroids such as Fluocinonide, Desoximetasone, Mometasone, Triamcinolone, Betamethasone, Alclometasone, Desonide, Hydrocortisone and Mapracorat; topical Calcineurin inhibitors such as Tacrolimus, pimecrolimus and cyclosporine; topical formulations of PDE4 inhibitors such as apremilast, E-6005, OPA-15406, LEO 29102, DRM02, and Roflumilast; topical formulations of JAK kinase inhibitors such as Tofacitinib, JTE-052, Baricitinib, and Upadacitinib; topical Non-steroidal anti-inflammatories such as WBI-1001, and MRX-6; antibiotic tetracycline agents such as minocycline and doxycycline, topical ROR agents such as GSK2981278; vitamin D analogs such as calcipotriene; oral Retinoic Acid derivatives such as alitretinoin; oral Liver X Receptor (LXR) selective agonists such as VTP-38543; oral H4 receptor antagonists such as ZPL-389; oral NK1 receptor antagonists such as Aprepitant and Tradipitant; oral CRTH2 receptor antagonists such as Fevipiprant and OC-459; oral Chymase inhibitors such as SUN 13834; oral GATA-3 inhibitors such as SB-011; oral ROR inverse agonists such as VTP-43742, ARN6039, TAK-828 and JTE-451; oral JAK inhibitors; including inhibitors of JAK1, JAK2, JAK3 and TYK2 such as PF-04965842, PF-06651600, and PF-06700841; oral PDE agents such as apremilast, roflumilast, and ibudilast; oral IRAK4 inhibitors such as PF-06650833; aryl hydrocarbon receptor antagonists such as tapinarof.

The term “co-administered” as used herein means that the second therapeutic agent may be administered together with Compound A, or a pharmaceutically acceptable salt thereof, as part of a single dosage form (such as a topical pharmaceutical composition of this disclosure) or as separate, multiple dosage forms. Alternatively, the additional agent may be administered prior to, consecutively with, or following the administration of Compound A, or a pharmaceutically acceptable salt thereof. In such combination therapy treatment, both the Compound A, or the pharmaceutically acceptable salt thereof, and the second therapeutic agent(s) are administered by conventional methods. The administration of a composition of this disclosure, comprising both Compound A, or a pharmaceutically acceptable salt thereof and a second therapeutic agent, to a subject does not preclude the separate administration of that same therapeutic agent, any other second therapeutic agent or Compound A, or a pharmaceutically acceptable salt of, to said subject at another time during a course of treatment.

### Kits

In some embodiments, the topical pharmaceutical compositions and methods described herein provide kits for the treatment of disorders or conditions, such as the ones described herein. These kits comprise a topical pharmaceutical composition described herein in a container and, optionally, instructions teaching the use of the kit according to the various methods and approaches described herein. Such kits may also include information, such as scientific literature references, package insert materials, clinical trial results, and/or summaries of these and the like, which indicate or establish the activities and/or advantages of the topical pharmaceutical composition, and/or which describe dosing, administration, side effects, drug interactions, or other information useful to the health care provider. Such information may be based on the results of various studies, for example, studies using experimental animals involving in-vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

Besides being useful for human treatment, topical pharmaceutical compositions of the disclosure, may be useful for veterinary treatment of companion animals, exotic animals and farm animals, including mammals, rodents, and the like. Conveniently, such animals include horses, dogs, and cats.

### Particular Methods of Treating an Inflammatory Skin Disorder

The present disclosure further provides a method for treating and/or preventing an inflammatory skin disorder in a subject in need thereof, the method comprising topically administering to the subject an effect amount of Compound A or a pharmaceutically acceptable salt thereof. In a further aspect, the composition is topically administered to an effected area of the subject's skin at an application dose of Compound A or a pharmaceutically acceptable salt thereof in an amount of from about 0.05 μg/cm2 to about 1000 μg/cm2 skin surface, about 0.05 g/cm2 to about 750 μg/cm2 skin surface, about 0.05 μg/cm2 to about 500 μg/cm2 skin surface, about 0.05 μg/cm2 to about 250 μg/cm2 skin surface, about 0.05 μg/cm2 to about 100 μg/cm2 skin surface, about 0.05 μg/cm2 to about 75 μg/cm2 skin surface, about 0.05 μg/cm2 to about 50 μg/cm2 skin surface, about 0.05 μg/cm2 to about 25 μg/cm2 skin surface, about 0.05 μg/cm2 to about 10 μg/cm2 skin surface, about 0.05 μg/cm2 to about 5 μg/cm2 skin surface, about 5 g/cm2 to about 1000 μg/cm2 skin surface, about 10 μg/cm2 to about 1000 μg/cm2 skin surface, about 25 μg/cm2 to about 1000 μg/cm2 skin surface, about 50 μg/cm2 to about 1000 g/cm2 skin surface, about 75 μg/cm2 to about 1000 μg/cm2 skin surface, about 100 μg/cm2 to about 1000 μg/cm2 skin surface, about 250 μg/cm2 to about 1000 μg/cm2 skin surface, about 500 μg/cm2 to about 1000 μg/cm2 skin surface, about 750 μg/cm2 to about 1000 μg/cm2 skin surface, about 5 μg/cm2 to about 750 μg/cm2 skin surface, about 10 μg/cm2 to about 500 g/cm2 skin surface, about 25 μg/cm2 to about 250 μg/cm2 skin surface, or about 50 μg/cm2 to about 100 μg/cm2 skin surface.

In various embodiments, provided herein is a dosing range of Compound A, or a pharmaceutically acceptable salt thereof, which is particularly useful for treating or preventing inflammatory skin disorders, such as psoriasis. There is therefore provided a method of treating or preventing an inflammatory skin disorder comprising administration of a topical pharmaceutical composition to a subject in need thereof, wherein the topical pharmaceutical composition comprises Compound A:

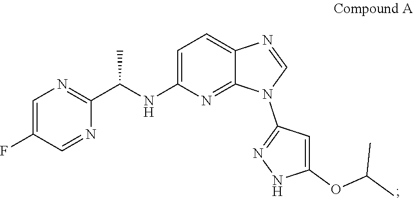 [SMILES: CC(C)Oc1cc(-n2cnc3ccc(N[C@@H](C)c4ncc(F)cn4)nc32)n[nH]1]

or a pharmaceutically acceptable salt thereof;
and wherein the composition is:
   a) applied topically to an affected area of the skin of a subject at an application dose of Compound A or a pharmaceutically acceptable salt thereof, in the range of 0.05 μg/cm² and 1000 μg/cm² skin surface; and
   b) the composition is administered in an amount sufficient to prevent or treat the inflammatory skin disorder in the subject.

The present disclosure further provides a composition for use in treating or preventing an inflammatory skin disorder comprising administration of a topical pharmaceutical composition to a subject in need thereof, wherein the topical pharmaceutical composition comprises Compound A:

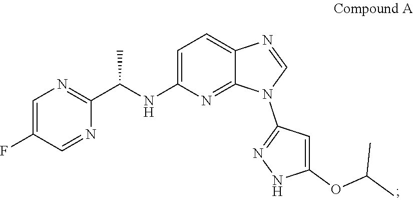 [SMILES: CC(C)Oc1cc(-n2cnc3ccc(N[C@@H](C)c4ncc(F)cn4)nc32)n[nH]1]

or a pharmaceutically acceptable salt thereof;
and wherein the composition is:
   a) applied topically to an affected area of the skin of a subject at an application dose of Compound A or a pharmaceutically acceptable salt thereof, in the range of 0.05 μg/cm² and 1000 μg/cm² skin surface; and
   b) the composition is administered in an amount sufficient to prevent or treat the inflammatory skin disorder in the subject.

Compound A has only ever clinically been evaluated in a Phase 1 trial in adults with recurrent glioblastoma multiforme (GBM). A summary of the study results was presented in Kreisl et. al. (2014) Neuro-Oncology 16:v79-v95. In this study, up to 60 mg of the compound was orally administered daily. No early efficacy signals in GBM patients were observed and the drug program was subsequently discontinued by the clinical trial sponsor, AstraZeneca Pharmaceuticals.

In 2015, it was reported that an investigational topical selective TRKA inhibitor, CT327, had been dosed in a Phase 2b clinical trial in patients with psoriasis (Roblin et. al. (2015) Acta Derm Venereol 2015; 95: 542-548). According to the report, no effect was found on psoriasis severity and no significant improvement in controlled disease response was seen for any dose of CT327 compared to vehicle. The authors concluded that the inhibition of keratinocyte hyperproliferation and sensory neuron function via TRKA kinase in the study may not be sufficient to alter the entire psoriatic disease state over 8 weeks of dosing, as measured by changes in either the IGA or mPASI instruments. This report thus teaches away from further investigation of TRK inhibition for the treatment of psoriasis.

In various embodiments, when dosed topically at low doses, Compound A, or a pharmaceutically acceptable salt thereof, can inhibit hyperproliferation of keratinocytes, which is a key feature of inflammatory skin disorders, such as psoriasis.

In various embodiments, a therapeutic window can be associated with topical dosing of Compound A for inflammatory skin disorders such as psoriasis. For example, as shown in Examples 11 and 12, a topical dosage range is demonstrated whereby the compound inhibits hyperproliferation of keratinocytes while minimizing toxicity and apoptosis of normal skin cells.

In some embodiments, the composition is applied topically to an affected area of the skin of a subject at an application dose of Compound A, or a pharmaceutically acceptable salt thereof in the range of 0.05 μg/cm² and 1000 μg/cm² skin surface. Conveniently, the topical pharmaceutical is applied topically to an affected area of the skin of a subject at an application dose of Compound A, or a pharmaceutically acceptable salt thereof in the range of 0.05 μg/cm² and 500 μg/cm² skin surface, such as in the range of 0.05 μg/cm² and 200 μg/cm², 0.1 μg/cm² and 200 μg/cm², 0.1 μg/cm² and 100 μg/cm², 0.1 μg/cm² and 10 μg/cm², or 0.5 μg/cm² and 5 μg/cm².

In some embodiments, the topical pharmaceutical composition comprises between 0.001% w/w and 5% w/w of Compound A, or a pharmaceutically acceptable salt thereof, conveniently between 0.005% w/w and 1% w/w, between 0.01% and 1% w/w or between 0.01% w/w and 0.1% w/w of Compound A, or a pharmaceutically acceptable salt thereof.

In some embodiments, the topical pharmaceutical composition comprises between 0.001% w/w and 5% w/w of Compound A, or a pharmaceutically acceptable salt thereof and the amount of said composition administered topically to the skin of the subject per application ranges between 0.05 mg and 1 g.

In some embodiments, the topical pharmaceutical composition comprises between 0.01% w/w and 0.1% w/w of Compound A, or a pharmaceutically acceptable salt thereof, and the amount of said composition administered topically to the skin of the subject per application ranges between 0.05 mg and 20 mg.

In some embodiments, the topical pharmaceutical composition comprises between 0.01% w/w and 0.1% w/w of Compound A or a pharmaceutically acceptable salt thereof and the amount of said composition administered topically to the skin of the subject per application ranges between 0.5 mg and 20 mg.

In some embodiments, the topical pharmaceutical composition is administered to the subject in need thereof between once and five times daily, such as once, twice, three, four or five times daily. In some embodiments, the topical pharmaceutical composition is administered to the subject in need thereof once daily.

In some embodiments, the total dose of Compound A, or a pharmaceutically acceptable salt thereof administered topically to the skin of the subject in a single day ranges between 0.05 mg and 2 g, such as between 0.05 mg and 400 mg, such as between 0.5 mg and 400 mg, or such as between 0.5 mg and 20 mg.

In some embodiments, the topical pharmaceutical composition is a cream, an ointment, a paste, a solution, a lotion, a gel, a rigid foam, a spray solution for spraying onto the skin, a transdermal patch, an adhesive strip, or a solid or a semi-solid composition.

In some embodiments, the topical pharmaceutical composition is a cream.

In some embodiments, the only active ingredient in the topical pharmaceutical composition is Compound A or a pharmaceutically acceptable salt thereof. In some embodiments, the pharmaceutically acceptable salt of Compound A is a mesylate salt. In some embodiments, the ratio of Compound A to methanesulfonic acid is about 1:1.

In some embodiments, the method comprises administration of a topical pharmaceutical composition of Compound A, or a pharmaceutical salt thereof, according to the first aspect disclosed herein.

In some embodiments, the method comprises administration of a topical pharmaceutical composition of Compound A, or a pharmaceutical salt thereof, wherein the pharmaceutical composition of Compound A comprises:
a) Compound A or a pharmaceutically acceptable salt thereof:
b) at least one permeation enhancer;
c) an antioxidant; and
d) one or more pharmaceutically acceptable excipients.

In some embodiments, the method comprises administration of a topical pharmaceutical composition of Compound A, or a pharmaceutical salt thereof, wherein the pharmaceutical composition of Compound A comprises:
a) a mesylate salt of Compound A:
b) at least one permeation enhancer;
c) an antioxidant;
d) a pH adjusting agent; and
e) one or more pharmaceutically acceptable excipients;
wherein the pH of the topical pharmaceutical composition at 25±1° C. is in the range of 4.5 and 7.

In some embodiments, the method comprises administration of a topical pharmaceutical composition of Compound A, or a pharmaceutical salt thereof, wherein the pharmaceutical composition of Compound A comprises:
a) between 0.005% w/w and 5% w/w of the mesylate salt of Compound A:
b) 3 to 40% w/w of the permeation enhancer;
c) 0.001% to 5.0% w/w of an antioxidant; and
d) a pH adjusting agent;
wherein weights are based on the total weight of the composition.

In some embodiments, the method comprises administration of a topical pharmaceutical composition of Compound A, or a pharmaceutical salt thereof, wherein the pharmaceutical composition of Compound A comprises:
a) between 0.005% w/w and 5% w/w of the mesylate salt of Compound A:
b) 3 to 40% w/w of diethylene glycol monoethyl ether;
c) 0.001% to 5.0% w/w butylated hydroxy toluene; and
d) triethanolamine;
wherein weights are based on the total weight of the composition.

In some embodiments, the method comprises administration of a topical pharmaceutical composition of Compound A, or a pharmaceutical salt thereof, wherein the pharmaceutical composition of Compound A comprises:
a) between 0.005% w/w and 0.2% w/w of the mesylate salt of Compound A:
b) 3 to 40% w/w of diethylene glycol monoethyl ether;
c) 0.001% to 5.0% w/w butylated hydroxy toluene; and
d) triethanolamine;
wherein weights are based on the total weight of the composition.

In some embodiments, the concentration of the Compound A or a pharmaceutically acceptable salt, achieved within the epidermis at 24 hours after administration is between 50 nM and 1.5 μM, more conveniently between 100 nM and 1.0 μM. The skilled person would be aware of techniques in which the amount of Compound A (or a pharmaceutically acceptable salt thereof) in the epidermis can be quantified. Suitable techniques include MALDI, OFM or Raman Spectroscopy.

In some embodiments, the geometric mean plasma concentration of Compound A at 12 hours (C₁₂) after the composition is administered topically to the skin of the subject in a single application is less than 5 ng/mL, such as less than 4 ng/mL, less than 3 ng/mL, less than 2 ng/mL, less than 1 ng/mL, or less than 0.5 ng/mL. Suitably, there is no detectable amount of Compound A at 12 hours (C₁₂) in the plasma after the composition is administered topically to the skin of the subject in a single application.

In some embodiments, the area under the plasma concentration-time curve from time zero up to 24 hours post-dose (AUC₀₋₂₄) after the composition is administered topically to the skin of the subject in a single application is less than approximately 100 ng·hr/mL.

### Inflammatory Skin Conditions

In some embodiments, the inflammatory skin disorder is associated with elevated expression or activity of a tropomyosin-related kinase (TRK). In some embodiments, the inflammatory skin disorder is associated with elevated expression or activity of tyrosine receptor kinase A (TRKA) and/or tyrosine receptor kinase B (TRKB) and/or tyrosine receptor kinase C (TRKC).

In some embodiments, the inflammatory skin disorder is selected from psoriasis, actinic keratosis, psoriasis guttata, inverse psoriasis, pustular psoriasis, psoriatic erythroderma, acute febrile neutrophilic dermatosis, eczema, xerotic eczema, dyshidrotic eczema, vesicular palmar eczema, acne vulgaris, atopic dermatitis, contact dermatitis, allergic contact dermatitis, dermatomyositis, exfoliative dermatitis, hand eczema, pompholyx, keloids, rosacea, rosacea due to sarcoidosis, rosacea due to scleroderma, rosacea due to Sweet syndrome, rosacea due to systemic lupus erythematosus, rosacea due to urticaria, rosacea due to herpetic pain, mastocytosis, uticaria, Sweet's disease, neutrophilic hydrodenitis, sterile pustule, drug rash, seborrheic dermatitis, Pityriasis rosea, Kikuchi's disease of the skin, pruritic urticarial papules and plaques of pregnancy, Stevens-Johnson syndrome and toxic epidermal necrolysis, tattoo reaction, Wells syndrome (eosinophilic cellulitis), reactive arthritis (Reiter syndrome), bowel-associated dermatosis-arthritis syndrome, rheumatoid neutrophilic dermatosis, neutrophilic eccrine hidradenitis, neutrophilic skin disease of dorsum of hand, balanitis circumscripta plasmacellularis, balanoposthitis, Behcet's disease, erythema annulare centrifugum, erythema dyschromicum perstans, erythema multiforme, granuloma annulare, dermatitis of hand, Lichen nitidus, Lichen planus, Lichen sclerosus et atrophicus, Lichen simplex chronicus, Lichen spinulosus, nummular dermatitis, pyoderma gangrenosum, sarcoidosis, subcorneal pustular dermatosis, urticaria, transient acantholytic dermatosis, bullous pemphigoid, dermatitis herpetiformis, dermatophytic infections, folliculitis, pediculosis, scabies, oral Lichen planus and aphthous ulcers.

In some embodiments, the inflammatory skin disorder is psoriasis or atopic dermatitis.

In some embodiments, the inflammatory skin disorder is psoriasis.

In some embodiments, the psoriasis is selected from plaque psoriasis, guttate psoriasis, inverse psoriasis, mild to moderate psoriasis, moderate to severe psoriasis, pustular psoriasis and erythrodermic psoriasis.

In some embodiments, the inflammatory skin disorder is psoriasis and administration of the composition is well tolerated with no dermal irritation or only minimal erythema.

In some embodiments, the inflammatory skin disorder is psoriasis and administration of the composition causes no dermal or neurological related adverse effects.

In some embodiments, the topical pharmaceutical composition is administered in combination with one or more additional therapeutic agents.

In some embodiments the topical pharmaceutical composition is administered in combination with topical corticosteroids, such as Fluocinonide, Desoximetasone, Mometasone, Triamcinolone, Betamethasone, Alclometasone, Desonide, Hydrocortisone and Mapracorat; JAK and/or TYK2 kinase inhibitors, such as Tofacitinib, JTE-052, Baricitinib, Upadacitinib, PF-04965842, PF-06651600 and PF-06700841; keratolytics, such as salicylic acid; antimicrobials; sunscreens; and antifungals.

### EXAMPLES

The following abbreviations have been used in the Examples:
API Active Pharmaceutical Ingredient
Eq.: equivalent
ETOAc: Ethyl acetate
LC: Label claim
LOQ: Limit of Quantification
NLT: Not less than
NMT: Not more than
RH: Relative humidity

### Example 1: Preparation of the mesylate salt of N-[(1S)-1-(5-fluoropyrimidin-2-yl)ethyl]-3-(5-isopropoxy-1H-pyrazol-3-yl)-3H-imidazo[4,5-b]pyridin-5-amine (Compound A Mesylate Salt)

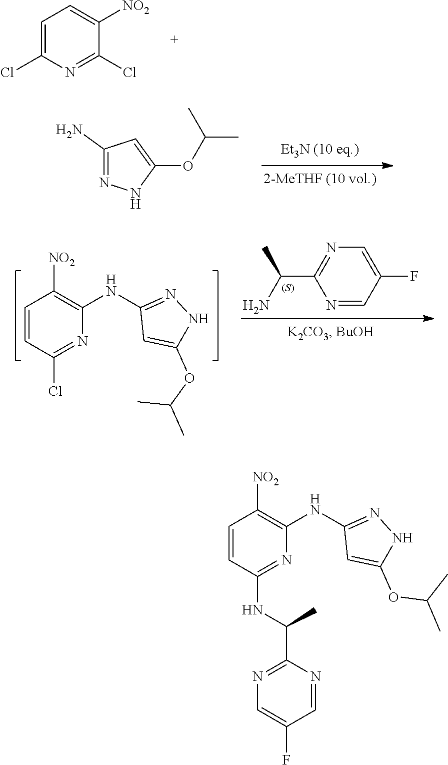

To a stirred solution of 2,6-dichloro-3-nitropyridine (1.0 eq.) and 5-propan-2-yloxy-1H-pyrazol-3-amine (1.1 eq.) in 2-MeTHF (10.0 vol.) triethyl amine (10.0 eq.) was added at 25-35° C. under a nitrogen atmosphere. After addition, the reaction mixture was stirred at 25-35° C. for 48 hours. The progress of the reaction was monitored by HPLC. After completion of the reaction, the reaction mixture was diluted with water (10 vol.) and the layers separated, and the organic layer was washed with 10.0 vol. of aqueous sodium chloride solution. Distillation of the solvent (2-Methyl-THF layer) at 45-55° C. under vacuum was carried out until 1-2 volumes remained in the round bottom flask, then co-distilled with 1-Butanol (2×2.5 volumes) and finally 10 volumes of 1-butanol were added to the mass. The crude mass was taken to the next stage as such without purification.

To a stirred solution of the above crude mass (1.0 eq.) in 1-butanol [(S)-1-(5-Fluoropyrimidin-2-yl)ethanamine (1.1 eq.) and potassium carbonate (2.5 eq.) were added at 25-35° C., followed by 2.5 volumes of 1-butanol. Then the reaction mass was heated to 70-75° C. for 24 hours. The progress of the reaction was monitored by HPLC. After completion of the reaction, the solid suspension was filtered and washed with 1-butanol (2×2 volumes). The wet material was dried at 50-55° C. for 12 hours and then slurried in 15 volumes of water. The solid suspension was stirred for 2 hours and filtered. The filtered cake was dried at 50-55° C. until the solid met the desired moisture content (Limit: 1%). If the isolated purity did not meet the specification limit (Limit: NLT 95%) a purification in MTBE was performed.

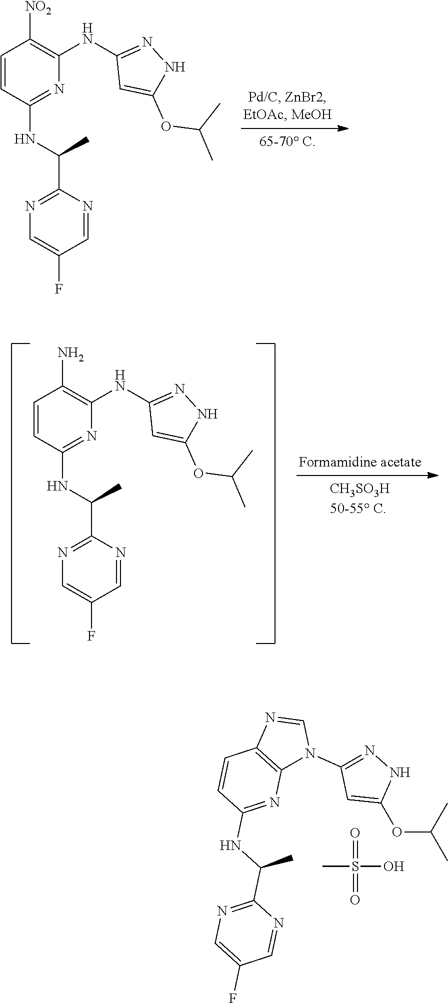

To a solution of (S)—N6-(1-(5-fluoropyrimidin-2-yl)ethyl)-N2-(5-isopropoxy-1H-pyrazol-3-yl)-3-nitropyridine-2,6-diamine (1.0 eq.), 10% Pd/C (50% wet, 15 wt %), formamidine acetate (2.0 eq.) and ZnBr₂ (0.2 eq.) in EtOAc (16.0 vol.), and MeOH (4.0 vol.) 4-6 kg/cm² of hydrogen gas was applied and the reaction mass was stirred at 65-70° C. for 4-5 hours. The progress of the reaction was monitored by HPLC. After completion of the reaction, the reaction mixture was cooled to 25-30° C. and the catalyst was removed by filtration under a nitrogen atmosphere and it was washed with EtOAc. The obtained organic layer was washed with brine solution (3×5 volumes) and the organic layer was treated with SC-40 charcoal and filtered, washed with EtOAc.

To a stirred solution of the above organic layer a solution of methane sulfonic acid (1.08 eq.) in water (0.15 vol.) was added at 55-60° C. Then reaction mass was maintained at 55-60° C. for 4 hours. Then reaction mass was slowly cooled to 5-10 C and filtered to obtain crude methane sulfonic acid salt of Compound A.

The crude methane sulfonic acid salt of Compound A was added to 1-butanol—water (3.3 eq and 0.7 eq) and heated to 50-55° C. Additional 5.5 eq 1-butanol was added and heated to 80-85° C. and stirred for 2 hours. The reaction mixture was filtered through a 0.2 micron filter and cooled to 25-30° C. over 12 hours. The slurry was cooled to 0-5° C., stirred for 6 hours and then filtered. The wet cake was washed with 1.9 eq butanol and dried at 50-55° C. for 12 hours to obtain methanesulfonic acid salt of Compound A, Form B.

The methane sulfonic acid salt of Compound A was added to 1-butanol—water (3.3 eq and 0.7 eq) and heated to 50-55° C. Additional 5.5 eq 1-butanol was added and heated to 80-85° C. and stirred for 2 hours. The reaction mixture was filtered through a 0.2 micron filter and cooled to 25-30° C. over 12 hours. The slurry was cooled to 0-5° C., stirred for 6 hours and then filtered. The wet cake was washed with 1.9 eq butanol and dried at 50-55° C. for 12 hours to obtain methanesulfonic acid salt of Compound A, Form B.

Filtered mother liquors were combined. Methy t-butyl ether (MTBE) (30 vol) was added and the mixture was stirred for 30-45 minutes at 25-30° C. The precipitated solid was filtered under vacuum and washed with MTBE (2.0 vol.) and the wet cake was dried for 2-3 hours at 40-50° C. to obtain the methane sulfonic acid salt of Compound A, Form A.

FIG. 1A shows a XRPD diffractogram of the methane sulfonic acid salt Form B.

FIG. 1B shows a XRPD diffractogram of the methane sulfonic acid salt Form A.

### Alternative Preparation of the Methane Sulfonic Acid Salt of Compound A, Form B

To a stirred solution of the methane sulfonic acid salt of Compound A (10 g, 1.0 eq.) in 1-Butanol (3.3 vol.), was added water (0.7 vol.) at 25-30° C. and the temperature was raised to 60° C. (at roughly 30° C. the solid started to dissolve forming a thin pale brown slurry and dark brown solution by 45° C.). 1-Butanol (5.5 vol.) was added drop wise into the reaction mass over a period of 20 minutes (Dark brown solution with small bits of suspension in it at 60° C.).

The reaction mass was heated to 80-85° C. and it was stirred at 80-85° C. for 1-2 hours.

The reaction mass was cooled to 58-62° C. and methane sulfonic acid salt of Compound A, Form B seed material (0.005 w/w) was added at 58-62° C. The reaction mass was stirred for 4-6 hours at 58-62° C. (Observation: Reaction mass becomes solid suspension).

The reaction mass was slowly cooled to 25-35° C. over a period of 10-12 hours. (Observation: Reaction mass is solid suspension).

Pre filtered 1-Butanol (1.9 vol.) was slowly added into the above RBF for a period of 1-2 hours at 25-35° C.

The reaction mass was stirred at 25-35° C. for 2-3 hours.

The reaction mass was cooled to 0-5° C. (Observation: Reaction mass is solid suspension).

The reaction mass was stirred at 0-5° C. for 5-6 hours and heated to 20-30° C. and then stirred at 20-30° C. for 12-14 hours. The reaction mass was further cooled to roughly 5° C. and it was stirred at this temperature for 2 hours.

The solid was filtered under vacuum and it was washed with 1-Butanol (1.9 vol.) and the wet cake was washed with MTBE (1.9 vol.). The wet cake was dried at 12-14 hours at 50° C. to get methane sulfonic acid salt of Compound A, Form B.

### Example 2: Solubility Determination of Compound a Mesylate Salt Form B in Excipients and Buffers

The solubility of N-[(1S)-1-(5-fluoropyrimidin-2-yl)ethyl]-3-(5-isopropoxy-1H-pyrazol-3-yl)-3H-imidazo[4,5-b]pyridin-5-amine mesylate (Form B) in individual excipients and buffers was determined after 24 hours at room temperature. The solubility was determined visually. The approximate amount of drug dissolved was calculated as mg/g of compound soluble in each solvent tested. The results are summarized in Table 1.

### Experimental Method

The solubility study was performed with addition of a known amount of Compound A mesylate salt in a known amount of solvent or solvent system until precipitation was observed that showed maximum solubility limits. The steps involved in performing the solubility study were as follows:
a) Label scintillation vials appropriately for each excipient that were tested.
b) Stir bar was placed in each vial and weighed the empty vial containing stir bar and cap with inert liner. Recorded the TARE weight.
c) Approximately 5.0 g of each excipient was weighed into the designated vial and approximately 5 mg of the API (Compound A mesylate salt) was added to into each vial.
d) All the closed vials were placed on a multi-position stir plate and allowed to mix for at least 15 minutes at ambient conditions.
e) Visually inspected to see if the API has completely dissolved.
f) If the API has dissolved, the last 3 steps were repeated. Additional API aliquots added to the mixture were recorded till saturation solubility reached. If the API is very soluble, the solubility was evaluated up to 12000 of the target concentration in the drug product. Stirring the solutions was continued overnight at ambient temperature to confirm solubility is maintained.
g) If API has not completely dissolved, continue stirring samples overnight at ambient conditions.
h) The final weight of the vial, cap, stir bar, and sample were recorded.
i) Further, the final observations were recorded as needed to document the observations.

TABLE 1
Solubility of Compound A mesylate salt (Form B)

| | | Amount | Amount | | |
|---|---|---|---|---|---|
| | | of | of API | | |
| | | excipient | (mg) | Solubility | |
| Ingredient | Function | added (g) | added | (mg/g) | Observation |
| Water | Solvent | 4.99 | 578.09 | >94.93 <115.61 | Precipitated at |
| | | | | | higher |
| | | | | | concentration |
| Transcutol P | Permeation | 5.01 | 47.25 | >4.17 <9.43 | Precipitated at |
| | enhancer | | | | higher |
| | | | | | concentration |
| Propylene glycol | | 5.03 | 188.74 | >22.34 <37.75 | Precipitated at |
| | | | | | higher |
| | | | | | concentration |
| Diisopropyl | | 5.02 | 4.93 | Minimum to | Precipitated- |
| adipate | | | | None | Uniform |
| | | | | | dispersion |
| IPM | | 5.01 | 4.91 | Minimum to | Precipitated- |
| (isopropyl | | | | None | Uniform |
| myristrate) | | | | | dispersion |
| Ethanol | | 5.01 | 33.05 | ≥6.61 | Solubilized |
| Dimethyl | Oil phase/ | 5.02 | 5.56 | Minimum to | Precipitated- |
| Isosorbide | Permeation | | | None | Uniform |
| | enhancer | | | | dispersion |
| Oleyl Alcohol | | 5.01 | 4.91 | Minimum to | Precipitated- |
| (HLB 13.5) | | | | None | Uniform |
| | | | | | dispersion |
| Medium Chain | | 5.01 | 5.00 | Minimum to | Precipitated- |
| Triglycerides | | | | None | Uniform |
| (HLB 15) | | | | | dispersion |
| Light Mineral Oil | Oil phase/ | 5.02 | 4.39 | Minimum to | Precipitated- |
| (HLB 4) | Occlusive | | | None | Uniform |
| | Agent/ | | | | dispersion |
| Cyclomethicone | Emollient | 5.02 | 6.58 | Minimum to | Precipitated- |
| | | | | None | Uniform |
| | | | | | dispersion |
| White petrolatum | | 5.05 | 5.01 | Minimum to | Precipitated- |
| (HLB 7) | | | | None | Uniform |
| | | | | | dispersion |
| PEG 400 | Solvent | 4.99 | 20.54 | >2.48 <4.12 | Precipitated at |
| | | | | | higher |
| | | | | | concentration |
| Glyceryl | Emulsifier/ | 5.00 | 16.09 | >1.01 <3.22 | Precipitated at |
| Monostearate | Thickening | | | | higher |
| (HLB 4.0) | agent | | | | concentration |
| Steareth-2 (HLB | | 4.97 | 26.32 | >1.03 <5.29 | Precipitated at |
| 4.9) | | | | | higher |
| | | | | | concentration |
| Steareth-20 (HLB | | 5.00 | 22.99 | >1.85 <4.59 | Precipitated at |
| 15.3) | | | | | higher |
| | | | | | concentration |
| Span 80 (HLB | | 5.00 | 4.68 | Minimum to | Precipitated- |
| 4.7) | | | | None | Uniform |
| | | | | | dispersion |
| Polysorbate 80 | | 5.00 | 66.16 | >7.09 <13.23 | Precipitated at |
| (HLB 15) | | | | | higher |
| | | | | | concentration |
| Tefose 63 (HLB | | 4.98 | 24.99 | >0.91 <5.02 | Precipitated at |
| 9.5) | | | | | higher |
| | | | | | concentration |
| Citrate buffer (pH | Buffers | 5.02 | 25.18 | >1.88 <5.02 | Precipitated at |
| 3.6) | | | | | higher |
| | | | | | concentration |
| Citrate buffer (pH | | 5.00 | 5.09 | Minimum to | Aggregated |
| 5.6) | | | | None | |
| Phosphate buffer | | 5.01 | 7.32 | Minimum to | Aggregated |
| (pH 7.4) | | | | None | |
| Glycerin | Humectant | 5.06 | 55.51 | >5.97 <10.96 | Precipitated at |
| | | | | | higher |
| | | | | | concentration |
| Sorbitol Solution | Texturizer | 5.01 | 17.64 | >1.0 <3.52 | Precipitated at |
| 70% | | | | | higher |
| | | | | | concentration |

As can be seen from Table 1, Compound A mesylate salt demonstrated water solubility of ˜9.5% w/w. It was also found that certain permeation enhancers solubilized the mesylate salt of the compound. Amongst the permeation enhancers, the mesylate salt demonstrated the highest solubility in propylene glycol. The salt was also soluble in Transcutol P and alcohol. The only suitable permeation enhancers that gave good solubility enhancement were Transcutol P, propylene glycol and ethanol. The salt had minimal to no solubility in the permeation enhancers IPM and diisopropyl adipate. Furthermore, the mesylate salt exhibited minimal or no solubility in various oils. The solubility in different buffers indicated that solubility decreased with increase in pH. Indeed, the compound precipitated in citrate pH 5.6 and phosphate pH 7.4 buffers. Higher concentrations (˜9.5% w/w) of Compound A mesylate salt in water resulted in light pink colored solutions.

### Example 3: Drug Excipient Compatibility

### Study Design

The goal of the drug excipient compatibility study was to identify excipients that are incompatible with the Compound A mesylate salt (Form B), resulting in high impurity growth compared to the baseline (To). The compatibility of the compound in selected excipients was assessed by mixing the compound with individual excipients and placing the mixtures on stability studies for 2 or 4 weeks at 25° C./60% RH and 40° C./75% RH. If the compound was insoluble in any excipient, the sample was prepared by first dissolving Compound A mesylate salt in a solvent (purified water) and then mixing with the excipient in a 1:1 ratio. A stock solution of the Compound A mesylate salt in water (0.45% w/w) was used. The total level of impurities over 4 weeks at the two stability conditions were measured. Samples also were evaluated for appearance, composition, and total impurities and the summary of results is presented in Table 2 and 3, respectively.

### Results

### Physical Appearance Summary

Physical appearance of Drug-Excipient mixture was evaluated visually, and the observations were recorded which are summarized in Table 2. Transcutol P, Propylene glycol, Oleyl alcohol, PEG 400 and Span 80 demonstrated significant change in colour at the end of the study. In contrast, only a slight change in colour was observed for ethanol, glycerin and sorbitol solution.

### Chemical Observation Summary

The chemical compatibility of different excipients is listed in Tables 3a and 3b. In summary, the compound was stable when mixed with the following chemicals: isopropyl myristate, ethanol, medium chain triglycerides, light mineral oil, cyclomethicone, white petrolatum, span 80, glycerin, sorbitol solution 70%, HPMC, HPC, and stearyl alcohol. Intermediate stability was observed with diisopropyl adipate, steareth-2, steareth-20, polysorbate 80, tefose 63, and cetyl alcohol. However, the mesylate salt of Compound A was found to degrade in transcutol P, propylene glycol, dimethyl isosorbide, oleyl alcohol, PEG-400, and glyceryl monostearate.

### Conclusions

It can be seen from Example 2 that Compound A mesylate salt was found to be soluble in certain permeation enhancers, e.g. Transcutol P, propylene glycol, water, and alcohol (ethanol/isopropyl alcohol). However, as demonstrated in the study in this Example 3, the Compound exhibits incompatibilities with many permeation enhancers (physical appearance and/or chemical observation) and excipients under accelerated storage conditions.

TABLE 2
Physical Observations

| | Observation | T2week | | T4week | |
|---|---|---|---|---|---|
| Sample | (T0) | 25° C./60% RH | 40° C./75% RH | 25° C./60% RH | 40° C./75% RH |
| Active only (A) | Pale Pink to | Pale Pink to | Pale Pink to | Pale Pink to | Pale Pink to |
| | off-white | off-white | off-white | off-white | off-white |
| | powder | powder | powder | powder | powder |
| Solvent system | Clear Liquid | Clear Liquid | Clear Liquid | Clear Liquid | Clear Liquid |
| only (S) | | | | | |
| A-S | Clear Liquid | Opaque/Turbid | Opaque/Turbid | Opaque/Turbid | Opaque/Turbid |
| | | Liquid | Liquid | Liquid | Liquid |
| (A-S): Transcutol | Clear Liquid | Light yellow | Yellow color | Light yellow | Yellow clear |
| P | | tinted liquid | clear liquid | liquid | liquid |
| (A-S): Propylene | Clear Liquid | Clear Liquid | Yellow color | Clear Liquid | Yellow clear |
| glycol | | | clear liquid | | liquid |
| (A-S): Di- | Opaque, Two | Clear, Two | Clear, Two | Clear, Two | Clear, Two |
| isopropyl adipate | phases | phases | phases | phases | phases |
| (A-S): Isopropyl | Clear, Two | Clear, Two | Clear, Two | Clear, Two | Clear, Two |
| Myristate | Phases | Phases | Phases | Phases | Phases |
| (A-S): Ethanol | Clear Liquid | Clear Liquid | Clear Liquid | Clear Liquid | Yellow clear |
| | | | | | liquid |
| (A-S): Dimethyl | Light yellow- | Light yellow | Light yellow | Light yellow | Light yellow |
| Isosorbide | Clear liquid | tinted liquid | tinted liquid | liquid | liquid |
| (A-S): Oleyl | Opaque, Two | Yellow to light | Dark yellow, | Golden yellow | Dark golden |
| Alcohol | phases | brown, two | two phases | liquid | yellow liquid |
| | | phases | | | |
| (A-S): Medium | Opaque, Two | Opaque, Two | Opaque, Two | Clear, two | Clear, two |
| Chain | phases | phases | phases | phases | phases |
| Triglycerides | | | | | |
| (A-S): Light | Clear, Two | Clear, Two | Clear, Two | Clear, Two | Clear, Two |
| Mineral Oil | Phases | Phases | Phases | Phases | Phases |
| (A-S): | Opaque, Two | Opaque, Two | Opaque, Two | Clear, two | Clear, two |
| Cyclomethicone | phases | phases | phases | phases | phases |
| (A-S): White | Solid opaque | Solid opaque | Solid opaque | Solid opaque | Solid opaque |
| petrolatum | mass not | mass not | mass not | mass not | mass not |
| | miscible in | miscible in | miscible in | miscible in | miscible in |
| | solvent | solvent | solvent | solvent | solvent |
| (A-S): PEG 400 | Clear Liquid | Clear Liquid | Yellow color | Light yellow | Yellow clear |
| | | | clear liquid | liquid | liquid |
| (A-S): Glyceryl | White solid | White solid | White solid | White solid | off white solid |
| Mono stearate | Mass | Mass | Mass | Mass | mass |
| (A-S): Steareth-2 | White solid | White solid | White solid | White solid | White solid |
| | mass | mass | mass | mass | mass |
| (A-S): Steareth- | Opaque | Opaque lumps | Opaque lumps | Opaque lumps | Opaque lumps |
| 20 | lumps | | | | |
| (A-S): Span 80 | Light yellow | Light yellow | Dark brown | Dark brown | Dark brown |
| | immiscible | immiscible | immiscible | immiscible | immiscible |
| | liquid | liquid | liquid | liquid | liquid |
| (A-S): | Yellow clear | Yellow clear | Yellow clear | Yellow clear | Yellow clear |
| Polysorbate 80 | high viscous | high viscous | high viscous | high viscous | high viscous |
| | liquid | liquid | liquid | liquid | liquid |
| (A-S): Tefose 63 | Off white to | Off white to | Off white to | Off white to | Off white to |
| | opaque solid | opaque solid | opaque solid | opaque solid | opaque solid |
| | mass | mass | mass | mass | mass |
| (A-S): Glycerin | Clear Liquid | Clear Liquid | Clear Liquid | Clear Liquid | Yellow tinted |
| | | | | | clear liquid |
| (A-S): Sorbitol | Clear Liquid | Clear Liquid | Clear Liquid | Clear Liquid | Light yellow |
| Solution | | | | | liquid |
| (A-S): HPMC | opaque gel | opaque gel | Opaque, yellow | Opaque, yellow | Opaque, yellow |
| | | | tinted | tinted | tinted |
| (A-S): HPC | opaque gel | opaque gel | Opaque, yellow | Opaque, yellow | Opaque, yellow |
| | | | tinted | tinted | tinted |
| (A-S): Cetyl | white solid | white solid | White solid | White solid | White solid |
| Alcohol | mass, | mass, | mass, light | mass, light | mass, yellow |
| | immiscible | immiscible | yellow tinted | yellow tinted | tinted liquid |
| | | | liquid | liquid | |
| (A-S): Stearyl | white solid | white solid | White solid | White solid | White solid |
| Alcohol | mass, | mass, | mass | mass | mass |
| | immiscible | immiscible | | | |
| S: Transcutol P | Clear Liquid | Clear Liquid | Clear Liquid | Clear Liquid | Clear Liquid |
| S: Propylene | Clear Liquid | Clear Liquid | Clear Liquid | Clear Liquid | Clear Liquid |
| glycol | | | | | |
| S: Di-isopropyl | Opaque, Two | Clear, Two | Clear, Two | Clear, Two | Clear, Two |
| adipate | phases | phases | phases | phases | phases |
| S: Isopropyl | Clear, Two | Clear, Two | Clear, Two | Clear, Two | Clear, Two |
| Myristate | Phases | Phases | Phases | Phases | Phases |
| S: Ethanol | Clear Liquid | Clear Liquid | Clear Liquid | Clear Liquid | Clear Liquid |
| S: Dimethyl | Clear Liquid | Clear Liquid | Clear Liquid | Clear Liquid | Clear Liquid |
| Isosorbide | | | | | |
| S: Oleyl Alcohol | Opaque, Two | Opaque, Two | Opaque, Two | Opaque, Two | Opaque, Two |
| | phases | phases | phases | phases | phases |
| S: Medium chain | Opaque, Two | Opaque, Two | Opaque, Two | Opaque, Two | Opaque, Two |
| triglycerides | phases | phases | phases | phases | phases |
| S: Light Mineral | Clear, Two | Clear, Two | Clear, Two | Clear, Two | Clear, Two |
| Oil | Phases | Phases | Phases | Phases | Phases |
| S: | Opaque, Two | Clear, Two | Clear, Two | Clear, Two | Clear, Two |
| Cyclomethicone | phases | phases | phases | phases | phases |
| S: White | Solid opaque | Solid opaque | Solid opaque | Solid opaque | Solid opaque |
| Petrolatum | mass | mass separated | mass separated | mass separated | mass separated |
| | separated | from solvent | from solvent | from solvent | from solvent |
| | from solvent | | | | |
| S: PEG 400 | Clear Liquid | Clear Liquid | Clear Liquid | Clear Liquid | Clear Liquid |
| S: Glyceryl | White solid | White solid | White solid | White solid | White solid |
| Monostearate | mass, | mass | mass, | mass, | mass, |
| | immiscible | immiscible | immiscible | immiscible | immiscible |
| S: Steareth-2 | White solid | White solid | White solid | White solid | White solid |
| | mass | mass | mass | mass | mass |
| S: Steareth-20 | Opaque | Opaque lumps | Opaque lumps | Opaque lumps | Opaque lumps |
| | lumps | | | | |
| S: Span 80 | Yellow liquid | Yellow liquid | Yellow liquid | Yellow liquid | Yellow liquid |
| | immiscible | immiscible | immiscible | immiscible | immiscible |
| S: Polysorbate 80 | Yellow clear | Yellow clear | Yellow clear | Yellow clear | Yellow clear |
| | high viscous | high viscous | high viscous | high viscous | high viscous |
| | liquid | liquid | liquid | liquid | liquid |
| S: Tefose 63 | Off white to | Off white to | Off white to | Off white to | Off white to |
| | opaque solid | opaque solid | opaque solid | opaque solid | opaque solid |
| | mass | mass | mass | mass | mass |
| S: Glycerin | Clear Liquid | Clear Liquid | Clear Liquid | Clear Liquid | Clear Liquid |
| S: Sorbitol | Clear Liquid | Clear Liquid | Clear Liquid | Clear Liquid | Clear Liquid |
| solution, 70% | | | | | |
| S: HPMC | Transparent | Transparent Gel | Transparent Gel | Transparent Gel | Transparent Gel |
| | Gel | | | | |
| S: HPC | Transparent | Transparent Gel | Transparent Gel | Transparent Gel | Transparent Gel |
| | Gel | | | | |
| S: Cetyl Alcohol | white solid | white solid | white solid | white solid | white solid |
| | mass, | mass, | mass, | mass, | mass, |
| | immiscible | immiscible | immiscible | immiscible | immiscible |
| S: Stearyl | white solid | white solid | white solid | white solid | white solid |
| Alcohol | mass, | mass, | mass, | mass, | mass, |
| | immiscible | immiscible | immiscible | immiscible | immiscible |

TABLE 3a
Chemical Observations 25° C./60% RH

| | | 25°C./60% RH | | | | | |
|---|---|---|---|---|---|---|---|
| | | T = 0 | | T = 2 weeks | | T = 4 weeks | |
| | | % | Total | % | Total | % | Total |
| Sample | | Label | Impurities | Label | Impurities | Label | Impurities |
| ID | Description | Claim | (%) | Claim | (%) | Claim | (%) |
| A1 | Active only (A) | 99.9 | 0.00 | 99.2 | 0.00 | 100.5 | 0.00 |
| A3 | A-S | 101.0 | 0.00 | 100.1 | 0.00 | 101.0 | 0.00 |
| A4 | (A-S): | 100.2 | 0.18 | 98.2 | 0.61 | 90.7 | 1.91 |
| | Transcutol P | | | | | | |
| A5 | (A-S): | 100.8 | 0.00 | 98.9 | 0.07 | 98.8 | 0.22 |
| | Propylene | | | | | | |
| | glycol | | | | | | |
| A6 | (A-S): | 75.4 | 0.00 | 67.0 | 0.08 | 65.8 | 0.12 |
| | Diisopropyl | | | | | | |
| | adipate | | | | | | |
| A7 | (A-S): | 100.4 | 0.00 | 99.2 | 0.00 | 99.7 | 0.00 |
| | Isopropyl | | | | | | |
| | Myristate | | | | | | |
| A8 | (A-S): Ethanol | 100.6 | 0.00 | 99.3 | 0.00 | 100.1 | 0.00 |
| A9 | (A-S): | 97.8 | 1.24 | 94.6 | 2.56 | 92.5 | 3.03 |
| | Dimethyl | | | | | | |
| | Isosorbide | | | | | | |
| A10 | (A-S): Oleyl | 90.2 | 0.89 | 78.4 | 1.28 | 76.0 | 2.23 |
| | Alcohol | | | | | | |
| A1 | (A-S): Medium | 97.3 | 0.00 | 95.5 | 0.00 | 96.2 | 0.00 |
| | Chain | | | | | | |
| | Triglycerides | | | | | | |
| A12 | (A-S): Light | 102.7 | 0.00 | 102.2 | 0.23 | 102.2 | 0.00 |
| | Mineral Oil | | | | | | |
| A13 | (A-S): | 103.4 | 0.00 | 101.7 | 0.00 | 102.0 | 0.00 |
| | Cyclomethicone | | | | | | |
| A14 | (A-S): White | 101.8 | 0.00 | 102.4 | 0.00 | 101.2 | 0.00 |
| | petrolatum | | | | | | |
| A15 | (A-S): PEG 400 | 99.3 | 0.25 | 96.7 | 1.40 | 96.0 | 1.88 |
| A16 | (A-S): Glyceryl | 97.8 | 0.05 | 87.7 | 0.14 | 67.4 | 0.15 |
| | Monosterate | | | | | | |
| A17 | (A-S): Steareth- | 97.9 | 0.00 | 62.6 | 0.00 | 78.3 | 0.06 |
| | 2 | | | | | | |
| A18 | (A-S): Steareth- | 99.9 | 0.14 | 96.5 | 0.15 | 99.4 | 0.21 |
| | 20 | | | | | | |
| A19 | (A-S): Span 80 | 95.6 | 0.00 | 97.5 | 0.00 | 96.2 | 0.11 |
| A20 | (A-S): | 98.4 | 0.00 | 95.7 | 0.52 | 97.9 | 0.68 |
| | Polysorbate 80 | | | | | | |
| A21 | (A-S): Tefose | 96.8 | 0.06 | 96.2 | 0.95 | 95.8 | 0.74 |
| | 63 | | | | | | |
| A22 | (A-S): Glycerin | 100.4 | 0.00 | 91.1* | 0.00 | 98.7 | 0.05 |
| A23 | (A-S): Sorbitol | 100.6 | 0.00 | 92.9* | 0.00 | 127.8* | 0.00 |
| | Solution 70% | | | | | | |
| A24 | (A-S): HPMC | 100.7 | 0.00 | 73.3* | 0.05 | 100.1 | 0.10 |
| A25 | (A-S): HPC | 100.6 | 0.07 | 98.9 | 0.10 | 96.2 | 0.08 |
| A26 | (A-S): Cetyl | 99.2 | 0.00 | 83.7 | 0.08 | 86.6 | 0.32 |
| | Alcohol | | | | | | |
| A27 | (A-S): Stearyl | 99.4 | 0.00 | 93.3* | 0.00 | 91.4 | 0.00 |
| | Alcohol | | | | | | |

TABLE 3b
Chemical Observations 40° C./75% RH

| | | 40° C./75% RH | | | | | | | |
|---|---|---|---|---|---|---|---|---|---|
| | | T = 0 | | T = 2 weeks | | | T = 4 weeks | | |
| | | % | Total | % | Total | | % | Total | |
| Sample | | Label | Impurities | Label | Impurities | Oxidation | Label | Impurities | Oxidation |
| ID | Description | Claim | (%) | Claim | (%) | Adjusted | Claim | (%) | Adjusted |
| A1 | Active only (A) | 99.9 | 0.00 | 99.7 | 0.00 | 0.00 | 99.1 | 0.00 | 0.00 |
| A3 | A-S | 101.0 | 0.00 | 100.6 | 0.00 | 0.00 | 101.1 | 0.21 | 0.21 |
| A4 | (A-S): | 100.2 | 0.18 | 88.2 | 4.78 | 1.23 | 89.5 | 5.72 | 1.72 |
| | Transcutol P | | | | | | | | |
| A5 | (A-S): | 100.8 | 0.00 | 89.9 | 3.12 | 1.47 | 88.2 | 4.10 | 1.98 |
| | Propylene | | | | | | | | |
| | glycol | | | | | | | | |
| A6 | (A-S) | 75.4 | 0.00 | 67.9 | 0.00 | 0.00 | 66.3 | 0.36 | 0.36 |
| | Diisopropyl | | | | | | | | |
| | adipate | | | | | | | | |
| A7 | (A-S): Isopropyl | 100.4 | 0.00 | 99.5 | 0.00 | 0.00 | 99.8 | 0.00 | 0.00 |
| | Myristate | | | | | | | | |
| A8 | (A-S): Ethanol | 100.6 | 0.00 | 99.6 | 0.07 | 0.00 | 93.0 | 2.34 | 1.24 |
| A9 | (A-S): Dimethyl | 97.8 | 1.24 | 91.4 | 3.86 | 0.46 | 89.9 | 5.08 | 1.01 |
| | Isosorbide | | | | | | | | |
| A10 | (A-S): Oleyl | 90.2 | 0.89 | 67.5 | 3.85 | 0.57 | 56.3 | 3.30 | 0.27 |
| | Alcohol | | | | | | | | |
| A11 | (A-S): Medium | 97.3 | 0.00 | 95.5 | 0.00 | 0.00 | 96.6 | 0.00 | 0.00 |
| | Chain | | | | | | | | |
| | Triglycerides | | | | | | | | |
| A12 | (A-S): Light | 102.7 | 0.00 | 102.5 | 0.00 | 0.00 | 102.4 | 0.16 | 0.16 |
| | Mineral Oil | | | | | | | | |
| A13 | (A-S): | 103.4 | 0.00 | 101.6 | 0.00 | 0.00 | 102.3 | 0.15 | 0.15 |
| | Cyclomethicone | | | | | | | | |
| A14 | (A-S): White | 101.8 | 0.00 | 100.1 | 0.00 | 0.00 | 101.6 | 0.17 | 0.17 |
| | petrolatum | | | | | | | | |
| A15 | (A-S): PEG 400 | 99.3 | 0.25 | 86.0 | 4.88 | 1.37 | 72.4 | 8.61 | 4.01 |
| A16 | (A-S): Glyceryl | 97.8 | 0.05 | 89.6 | 1.08 | 0.72 | 83.3 | 1.85 | 1.13 |
| | Monosterate | | | | | | | | |
| A17 | (A-S): Steareth-2 | 97.9 | 0.00 | 64.4 | 0.08 | 0.00 | 67.6 | 0.54 | 0.30 |
| A18 | (A-S): Steareth-20 | 99.9 | 0.14 | 97.8 | 0.49 | 0.06 | 98.0 | 0.81 | 0.06 |
| A19 | (A-S): Span 80 | 95.6 | 0.00 | 95.6 | 0.00 | 0.00 | 97.6 | 0.54 | 0.54 |
| A20 | (A-S): Polysorbate 80 | 98.4 | 0.00 | 95.6 | 1.43 | 0.00 | 91.7 | 2.86 | 0.24 |
| A21 | (A-S): Tefose 63 | 96.8 | 0.06 | 98.2 | 0.55 | 0.22 | 93.1 | 1.39 | 0.70 |
| A22 | (A-S): Glycerin | 100.4 | 0.00 | 100.8 | 0.11 | 0.00 | 97.3 | 0.82 | 0.16 |
| A23 | (A-S): Sorbitol | 100.6 | 0.00 | 99.2 | 0.10 | 0.05 | 98.7 | 0.37 | 0.21 |
| | Solution 70% | | | | | | | | |
| A24 | (A-S): HPMC | 100.7 | 0.00 | 98.8 | 0.13 | 0.00 | 98.2 | 0.71 | 0.15 |
| A25 | (A-S): HPC | 100.6 | 0.07 | 99.1 | 0.22 | 0.00 | 99.7 | 0.19 | 0.00 |
| A26 | (A-S): Cetyl Alcohol | 99.2 | 0.00 | 78.0 | 0.82 | 0.26 | 88.0 | 1.25 | 0.35 |
| A27 | (A-S): Stearyl Alcohol | 99.4 | 0.00 | 97.5 | 0.19 | 0.14 | 82.1 | 0.00 | 0.00 |

### Example 4. Effects of Antioxidants

The study described in Example 3 demonstrated that when Compound A mesylate salt is tested as a binary mixture (API/excipient) as described above, Compound A mesylate salt exhibits a level of incompatibility with certain excipients, for example certain permeation enhancers.

The effects of antioxidants on the stability of the Compound A mesylate salt (Form B) with different excipients were evaluated. Samples were stored at 25° C./60% RH for 1 week and 40° C./75% RH for two weeks and were tested at one week (T0) and two weeks (T2) respectively. The results of the tests are shown in Table 4 below. 003171 Stock solutions of the antioxidants in purified water and in benzyl alcohol were mixed with N-[(1S)-1-(5-fluoropyrimidin-2-yl)ethyl]-3-(5-isopropoxy-1H-pyrazol-3-yl)-3H-imidazo[4,5-b]pyridin-5-amine mesylate Form B (Compound A mesylate) at 0.500 w/w dissolved in an excipient (water, phenoxyethanol, Transcutol HP, propylene glycol 400 SR, polyethylene glycol SR, or benzyl alcohol) and placed on stability for two weeks.

TABLE 4
Antioxidant summary

| | | | | T2W (2 weeks | |
|---|---|---|---|---|---|
| | | T0 (1 week at RT) | | at 40° C.) | |
| | | | Total | | Total |
| | | % | Im- | % | Impu- |
| Anti- | | Label | purities | Label | rities |
| oxidant | Excipient | Claim | (%) | Claim | (%) |
| No | | 99.70 | <LOQ | 98.62 | <LOQ |
| Anti- | Water | 99.33 | <LOQ | 99.15 | 0.13 |
| oxidant | Phenoxyethanol | 101.86 | <LOQ | 99.15 | 0.10 |
| | Transcutol HP | 93.79 | 3.06 | 56.56 | 26.02 |
| | Propylene Glycol | 95.98 | <LOQ | 96.42 | <LOQ |
| | SR | | | | |
| | Polyethylene | 96.06 | 0.82 | 95.25 | 2.05 |
| | Glycol 400 SR | | | | |
| | Benzyl Alcohol | 94.12 | 3.19 | 91.6 | 6.88 |
| TPGS- | Water | 99.02 | 0.14 | 98.96 | 0.19 |
| Vit E | | | | | |
| Succinate | | | | | |
| BHT | Phenoxyethanol | 100.58 | <LOQ | 99.38 | 0.08 |
| | Transcutol HP | 100.56 | 0.06 | 99.36 | 0.20 |
| | Propylene Glycol | 99.08 | <LOQ | 99.74 | 0.07 |
| | SR | | | | |
| | Polyethylene | 96.68 | 0.84 | 97.11 | 0.84 |
| | Glycol 400 SR | | | | |
| | Benzyl Alcohol | 97.55 | 2.47 | 93.65 | 8.29 |
| BHA | Phenoxyethanol | 100.62 | <LOQ | 99.87 | 0.51 |
| | Transcutol HP | 104.72 | <LOQ | 104.68 | 0.24 |
| | Propylene Glycol | 99.59 | <LOQ | 99.27 | 0.57 |
| | SR | | | | |
| | Polyethylene | 98.25 | 0.82 | 93.04 | 3.20 |
| | Glycol 400 SR | | | | |
| | Benzyl Alcohol | 97.53 | 2.64 | 98.29 | 3.16 |
| BHT + | Phenoxyethanol | 99.03 | <LOQ | 98.84 | 1.31 |
| BHA | Transcutol HP | 99.11 | <LOQ | 99.08 | <LOQ |
| | Propylene Glycol | 99.72 | <LOQ | 98.12 | 0.42 |
| | SR | | | | |
| | Polyethylene | 96.83 | 0.80 | 94.20 | 7.18 |
| | Glycol 400 SR | | | | |
| | Benzyl Alcohol | 95.47 | 2.52 | 95.65 | 3.61 |
| Vitamin | Phenoxyethanol | 100.11 | <LOQ | 99.51 | 0.08 |
| E | Transcutol HP | 95.00 | 2.62 | 62.07 | 20.72 |
| | Propylene Glycol | 99.29 | <LOQ | 99.43 | <LOQ |
| | SR | | | | |
| | Polyethylene | 98.80 | 0.89 | 96.60 | 1.90 |
| | Glycol 400 SR | | | | |
| | Benzyl Alcohol | 98.79 | 1.20 | 78.40 | 22.68 |
| Vitamin | Phenoxyethanol | 99.98 | <LOQ | 99.49 | 0.09 |
| E | Transcutol HP | 96.07 | 2.00 | 62.96 | 23.73 |
| Acetate | Propylene Glycol | 98.84 | <LOQ | 98.24 | 0.05 |
| | SR | | | | |
| | Polyethylene | 97.80 | 0.92 | 95.82 | 2.35 |
| | Glycol 400 SR | | | | |
| | Benzyl Alcohol | 98.40 | 1.32 | 93.29 | 7.97 |

Table 4 shows the results of the antioxidant evaluation.

Controls: The control tests show there was minimal degradation (<0.50%) observed of Compound A mesylate salt in either water, phenoxyethanol or propylene glycol. Significant degradation was observed in the control tests for PEG 400 SR, benzyl alcohol, and Transcutol HP. For propylene glycol it is noted that some chemical instability was observed during the drug excipient compatibility study described in Example 3 above.

Transcutol HP: oil-soluble antioxidants BHT, BHA alone or in combination were effective in minimizing degradation from ˜26% impurities in control to <0.5% o. In contrast, Vitamin E and Vitamin E acetate were ineffective (>20%).

Polyethylene Glycol 400 SR: BHT was partially effective in minimizing degradation from ˜2% impurities to 0.8%. BHA and BHT+BHA caused an increase in impurities relative to the control. No change in degradation levels were observed with Vitamin E or Vitamin E acetate compared to the control tests.

Benzyl Alcohol: All antioxidants tested were ineffective at minimizing degradation of the Compound. Vitamin E was incompatible with benzyl alcohol as the total impurities increased to ˜23% versus ˜6% in the control.

Phenoxyethanol: No changes in impurity levels observed in the presence of BHT, vitamin E or vitamin E acetate compared to the control. Total impurity levels increased in the presence of BHA and BHT+BHA compared to the control.

Propylene Glycol SR: No changes in impurity levels observed in the presence of BHT, vitamin E or vitamin E acetate compared to the control. Total impurity levels increased in the presence of BHA and BHT+BHA compared to the control.

Water: Minimal degradation was observed in the presence of TPGS-Vitamin E succinate compared to the control.

### Conclusions

Surprisingly, only certain antioxidants prevented degradation of the compound in particular excipients. The oil soluble antioxidants (e,g., BHA and BHT) were particularly effective for Transcutol HP.

### Example 5: Exemplary Compositions

Exemplary formulations of the present disclosure containing 1% w/w of Compound A mesylate salt (Form B) were prepared with ingredients described in Table 5.

In addition to the compositions described in Table 5, attempts to prepare gel based topical compositions were also made. Various polymers and gelling agents were screened, including hydroxypropyl cellulose, hydroxyethyl cellulose, Sepineo P 600 (Acrylamide/Sodium acryloyldimethyl taurate Copolymer/Isohexadecane & Polysorbate 80), carbomers (homopolymer acrylic acids) but the gels produced were of poor quality. It is believed that the physical instability observed may have been due to the salt content of the compositions and possible interactions between the Compound A mesylate salt and certain polymers.

TABLE 5

| Formulation | | | | | | | | | | F10, |
|---|---|---|---|---|---|---|---|---|---|---|
| No. | F1, 1% | F2, 1% | F3, 1% | F4, 1% | F5, 1% | F6, 1% | F7, 1% | F8, 1% | F9, 1% | 1% |
| Ingredients | Amount (% w/w) | | | | | | | | | |
| Compound A | 1.0 | 1.0 | 1.0 | 1.0 | 1.0 | 1.0 | 1.0 | 1.0 | 1.0 | 1.0 |
| mesylate salt, | | | | | | | | | | |
| Form B | | | | | | | | | | |
| Water | 66.85 | 59.05 | 62.95 | 64.05 | 66.05 | 46.05 | 39.05 | 42.05 | 25.05 | |
| Transcutol HP | | | | 10.0 | | 10.0 | 10.0 | 10.0 | 10.0 | 10.0 |
| Propylene | | 5.0 | | | | | | | | |
| glycol SR | | | | | | | | | | |
| Isopropyl | | | 10.0 | | | | | | | |
| myristate | | | | | | | | | | |
| Oleyl alcohol | | | | | 10.0 | | 8.0 | 3.0 | | |
| Octyldodecanol | | | 8.0 | | | | | 5.0 | | |
| Medium Chain | 10.0 | | | | | 6.0 | | | | |
| Triglycerides | | | | | | | | | | |
| Cyclomethicone | | 6 | | 3 | | 7 | 5 | 5.0 | 5.0 | |
| Dimethicone | 5 | | | | | | | 3 | | 5 |
| White | | 7 | | | 8 | 3 | 10 | 5 | 25 | 70.5 |
| petrolatum | | | | | | | | | | |
| Cetostearyl | | | | | | | 12 | | | |
| alcohol | | | | | | | | | | |
| Cetyl alcohol | | 5.0 | | | 4 | 2.0 | | 4.0 | | |
| Stearyl alcohol | 4 | | | | | 2.0 | | 4.0 | 7.0 | |
| Glyceryl | | 5 | 3 | | | 5 | 4 | 3 | 5 | 3.9 |
| Monosterate | | | | | | | | | | |
| Type II | | | | | | | | | | |
| Paraffin wax | | | | | | | | | 5 | |
| White beeswax | | | | | | | | | 5 | |
| Cetomacrogol | 4.1 | | | 5 | | | | | | |
| 1000 | | | | | | | | | | |
| PPG-15 Stearyl | 0.1 | | 4.1 | 3 | | | | | | |
| ether | | | | | | | | | | |
| Stearic acid | | | | | | | 3 | | | |
| Steareth-2 | | | | | 5 | | | 4 | | |
| Steareth-20 | | | | | | 4 | | | | |
| Span 60 | | 4 | | | 4 | | | 4 | | |
| Span 80 | 7 | | | | | | | | | |
| Polyoxy-40- | | | | | | | | | 5 | |
| stearate | | | | | | | | | | |
| Polysorbate 80 | | | | | | | | | | 3 |
| PEG-100 | | | | | | | 1 | | | |
| Stearate | | | | | | | | | | |
| Sodium | | | 5 | 3 | | | | | | 5 |
| cetosteary1 | | | | | | | | | | |
| sulfate | | | | | | | | | | |
| Glycerine | | | 4 | | | | | | | |
| Sepineo P600 | | | | 1 | | | | | | |
| Hydroxypropyl | | | | 5 | | 5 | | | | |
| Cellulose | | | | | | | | | | |
| Sorbitol | 0.1 | 0.1 | 0.1 | 0.1 | 0.1 | 0.1 | 0.1 | 0.1 | 0.1 | 0.1 |
| Solution 70% | | | | | | | | | | |
| Butylated | 0.1 | 0.1 | 0.1 | 0.1 | 0.1 | 0.1 | 0.1 | 0.1 | 0.1 | |
| hydroxytoluene | | | | | | | | | | |
| Sorbic acid | 0.18 | 0.18 | 0.18 | 0.18 | 0.18 | 0.18 | 0.18 | 0.18 | 0.18 | |
| Methyl paraben | 0.02 | 0.02 | 0.02 | 0.02 | 0.02 | 0.02 | 0.02 | 0.02 | 0.02 | |
| Propyl paraben | 0.05 | 0.05 | 0.05 | 0.05 | 0.05 | 0.05 | 0.05 | 0.05 | 0.05 | |
| Disodium | 1.5 | 1.5 | 1.5 | 1.5 | 1.5 | 1.5 | 1.5 | 1.5 | 1.5 | 1.5 |
| edetate | | | | | | | | | | |
| Trolamine | | 6 | | 3 | | 7 | 5 | 5 | 5 | |

Further formulations containing 300 w/w of Compound A mesylate salt (Form B) were prepared with the ingredients described in Table 6. The stability of these exemplary formulations were tested, and the results after 1 month are shown in Table 7.

TABLE 6

| Formulation | F1, | F2, | F3, | F4, | F5, | F6, | F7, | F8, | F9, | F10, |
|---|---|---|---|---|---|---|---|---|---|---|
| No. | 3% | 3% | 3% | 3% | 3% | 3% | 3% | 3% | 3% | 3% |
| Ingredients | Amount (% w/w) | | | | | | | | | |
| Compound A | 3 | 3 | 3 | 3 | 3 | 3 | 3 | 3 | 3 | 3 |
| mesylate salt, Form | | | | | | | | | | |
| B | | | | | | | | | | |
| Water | 63.35 | 61.55 | 59.45 | 63.55 | 57.55 | 49.55 | 41.55 | 46.55 | 26.55 | |
| Transcutol HP | | | | 10 | | 10 | 10 | 10 | 10 | 10 |
| Propylene glycol SR | | 5 | | | | | | | | |
| Isopropyl myristate | | | 10 | | | | | | | |
| Oleyl alcohol | | | | | 10 | | 8 | 3 | | |
| Octyldodecanol | | | 8 | | | | | 5 | | |
| Medium Chain | 10 | | | | | 6 | | | | |
| Triglycerides | | | | | | | | | | |
| Cyclomethicone | | 6 | | 3 | | 7 | 8 | 5 | 5 | |
| Dimethicone | 5 | | | | | | | | | 5 |
| White petrolatum | | 7 | | | 8 | 3 | 10 | 5 | 25 | 67 |
| Cetostearyl alcohol | | | | | | | 3 | | | |
| Cetyl alcohol | | 5 | | | 4 | 2 | | 4 | | |
| Stearyl alcohol | 4 | | | | | 2 | | 4 | 7 | |
| Glyceryl | | 5 | 3 | | | 5 | 6 | 3 | 5 | 3.9 |
| Monosterate Type II | | | | | | | | | | |
| Paraffin wax | | | | | | | | | 5 | |
| White beeswax | | | | | | | | | | 3 |
| Cetomacrogol 1000 | | | | | | | | | 5 | |
| PPG-15 Stearyl ether | | | | | | | | | 5 | |
| Stearic acid | | | | | 4 | | | | | |
| Steareth-2 | 4.1 | | | 5 | | | | | | |
| Steareth-20 | 0.1 | | 4.1 | 3 | | | | | | |
| Span 60 | | | | | 6 | | 3 | | | |
| Span 80 | | | | | | | | 4 | | |
| Polyoxy-40-stearate | | | | | | 4 | 4 | | | |
| Polysorbate 80 | | 4 | | | 4 | | | 4 | | |
| PEG-100 Stearate | 7 | | | | | | | | | |
| Sodium cetostearyl | | | | | | | | | | |
| sulfate | | | | | | | | | | |
| Glycerine | | | 5 | 3 | | | | | | 5 |
| Sepineo P600 | | | 4 | | | | | | | |
| Hydroxypropyl | | | | 1 | | | | | | |
| cellulose | | | | | | | | | | |
| Sorbitol Solution | | | | 5 | | 5 | | | | |
| 70% | | | | | | | | | | |
| Butylated | 0.1 | 0.1 | 0.1 | 0.1 | 0.1 | 0.1 | 0.1 | 0.1 | 0.1 | 0.1 |
| hydroxytoluene | | | | | | | | | | |
| Sorbic acid | 0.1 | 0.1 | 0.1 | 0.1 | 0.1 | 0.1 | 0.1 | 0.1 | 0.1 | |
| Methyl paraben | 0.18 | 0.18 | 0.18 | 0.18 | 0.18 | 0.18 | 0.18 | 0.18 | 0.18 | |
| Propyl paraben | 0.02 | 0.02 | 0.02 | 0.02 | 0.02 | 0.02 | 0.02 | 0.02 | 0.02 | |
| Disodium edetate | 0.05 | 0.05 | 0.05 | 0.05 | 0.05 | 0.05 | 0.05 | 0.05 | 0.05 | |
| Trolamine | 3 | 3 | 3 | 3 | 3 | 3 | 3 | 3 | 3 | 3 |

TABLE 7

| | | | | | Related Substances (% R.S) | | | | | |
|---|---|---|---|---|---|---|---|---|---|---|
| | | | | | | | | | RRT | |
| | | | | | | | | | 2.300/ | |
| Sample | Test Time | | | Assay | RRT | RRT | RRT | RRT | RRT | |
| No. | (Months) | Condition | pH | (% L.C.) | 0.734 | 0.746 | 0.884 | 1.093 | 2.345 | Total |
| F1,3% | 0 | | 3.03 | 103.5 | <LOQ | <LOQ | <LOQ | <LOQ | ND | 0.00 |
| Cream | 1 | 25° C./ | 3.06 | 104.4 | <LOQ | <LOQ | <LOQ | <LOQ | ND | 0.00 |
| | | 60% RH | | | | | | | | |
| | | 40° C./ | 3.00 | 110.9 | <LOQ | <LOQ | <LOQ | <LOQ | ND | 0.00 |
| | | 75% RH | | | | | | | | |
| F2,3% | 0 | | 3.00 | 105.4 | <LOQ | <LOQ | <LOQ | <LOQ | ND | 0.00 |
| Cream | 1 | 25° C./ | 2.99 | 106.9 | <LOQ | <LOQ | <LOQ | <LOQ | ND | 0.00 |
| | | 60% RH | | | | | | | | |
| | | 40° C./ | 2.98 | 102.7 | <LOQ | <LOQ | <LOQ | <LOQ | ND | 0.00 |
| | | 75% RH | | | | | | | | |
| F3, 3% | 0 | | 2.78 | 104.1 | <LOQ | <LOQ | <LOQ | <LOQ | 0.59 | 0.59 |
| Cream | 1 | 25° C./ | 2.82 | 104.6 | <LOQ | <LOQ | <LOQ | <LOQ | 1.02 | 1.02 |
| | | 60% RH | | | | | | | | |
| | | 40° C./ | 2.78 | 105.1 | <LOQ | <LOQ | <LOQ | <LOQ | 1.02 | 1.02 |
| | | 75% RH | | | | | | | | |
| F4,3% | 0 | | 3.35 | 110.2 | <LOQ | <LOQ | <LOQ | <LOQ | ND | 0.00 |
| Cream | 1 | 25° C./ | 3.43 | 110.5 | <LOQ | <LOQ | <LOQ | <LOQ | ND | 0.00 |
| | | 60% RH | | | | | | | | |
| | | 40° C./ | 3.25 | 108.4 | <LOQ | <LOQ | 0.05 | <LOQ | ND | 0.05 |
| | | 75% RH | | | | | | | | |
| F5, 3% | 0 | | 2.76 | 106.7 | <LOQ | <LOQ | <LOQ | <LOQ | ND | 0.00 |
| Cream | 1 | 25° C./ | 2.63 | 106.0 | <LOQ | <LOQ | <LOQ | <LOQ | ND | 0.00 |
| | | 60% RH | | | | | | | | |
| | | 40° C./ | 2.71 | 115.7 | <LOQ | <LOQ | <LOQ | <LOQ | ND | 0.00 |
| | | 75% RH | | | | | | | | |
| F6, 3% | 0 | | 3.32 | 99.6 | <LOQ | <LOQ | <LOQ | <LOQ | ND | 0.00 |
| Cream | 1 | 25° C./ | 3.15 | 100.0 | <LOQ | <LOQ | <LOQ | <LOQ | ND | 0.00 |
| | | 60% RH | | | | | | | | |
| | | 40° C./ | 3.24 | 100.4 | <LOQ | <LOQ | 0.05 | <LOQ | ND | 0.05 |
| | | 75% RH | | | | | | | | |
| F7, 3% | 0 | | 3.27 | 105.3 | <LOQ | <LOQ | <LOQ | <LOQ | ND | 0.00 |
| Cream | 1 | 25° C./ | 3.22 | 105.4 | <LOQ | <LOQ | <LOQ | <LOQ | ND | 0.00 |
| | | 60% RH | | | | | | | | |
| | | 40° C./ | 3.26 | 105.9 | <LOQ | <LOQ | <LOQ | <LOQ | ND | 0.00 |
| | | 75% RH | | | | | | | | |
| F8, 3% | 0 | | 3.11 | 103.4 | <LOQ | <LOQ | <LOQ | <LOQ | ND | 0.00 |
| Cream | 1 | 25° C./ | 3.13 | 103.0 | <LOQ | <LOQ | <LOQ | <LOQ | ND | 0.00 |
| | | 60% RH | | | | | | | | |
| | | 40° C./ | 3.14 | 103.2 | <LOQ | <LOQ | <LOQ | <LOQ | ND | 0.00 |
| | | 75% RH | | | | | | | | |
| F9, 3% | 0 | | 3.53 | 101.6 | <LOQ | <LOQ | <LOQ | <LOQ | ND | 0.00 |
| Ointment | 1 | 25° C./ | 3.55 | 102.0 | <LOQ | <LOQ | <LOQ | <LOQ | ND | 0.00 |
| | | 60% RH | | | | | | | | |
| | | 40° C./ | 3.51 | 102.6 | <LOQ | <LOQ | <LOQ | <LOQ | ND | 0.00 |
| | | 75% RH | | | | | | | | |
| F10, 3% | 0 | | 4.51 | 96.2 | <LOQ | <LOQ | <LOQ | <LOQ | ND | 0.00 |
| Ointment | 1 | 25° C./ | 3.84 | 97.4 | <LOQ | <LOQ | <LOQ | <LOQ | ND | 0.00 |
| | | 60% RH | | | | | | | | |
| | | 40° C./ | 3.82 | 95.7 | <LOQ | <LOQ | <LOQ | <LOQ | ND | 0.00 |
| | | 75% RH | | | | | | | | |

The results in Table 7 show that the compound was found to be chemically stable at the 1 month time point in the compositions tested. Formulation F3 was the only composition that contained greater than 1% o impurity levels under accelerated conditions.

At the one month time point, some of the formulations presented physical instability with phase separation and pH drop towards acidic behavior. For some of the compositions, it was surprisingly found that the pH drop could be avoided by altering the Trolamine addition step. Formulations F1, F6, F8, and F9 containing 1 or 3% o w/w of Compound A mesylate salt were remanufactured as “pH optimized” or “pH opt” using Trolamine (diluted 250 w/w) titration to achieve the accepted pH of 51, see Table 8.

Additional exemplary formulations of the present disclosure containing 300 w/w of Compound A (Form B) were also prepared with ingredients described in Table 8.

TABLE 8

| | F1, 1% | F1, 3% | F6, 1% | F6, 3% | F8,1% | F8, 3% | F9, 1% | F9, 3% |
|---|---|---|---|---|---|---|---|---|
| Formulation | pH opt | pH opt | pH opt | pH opt | pH opt | pH opt | pH opt | pH opt |
| Ingredients | Amount (% w/w) | | | | | | | |
| Compound A | 1.0 | 3.0 | 1.0 | 3.0 | 1.0 | 3.0 | 1.0 | 3.0 |
| mesylate salt, | | | | | | | | |
| Form B | | | | | | | | |
| Water, Purified | 67.05 | 62.35 | 42.25 | 37.55 | 44.25 | 39.55 | 25.25 | 20.55 |
| Transcutol HP | | | 10.0 | 10.0 | 10.0 | 10.0 | 10.0 | 10.0 |
| Oleyl Alcohol | | | | | 3.0 | 3.0 | | |
| Octyldodecanol | | | | | 5.0 | 5.0 | | |
| Medium Chain | 10.0 | 10.0 | 10.0 | 10.0 | | | | |
| Triglycerides | | | | | | | | |
| Cyclomethicone | | | | | 5.0 | 5.0 | 5.0 | 5.0 |
| Dimethicone | 5.0 | 5.0 | | | 3.0 | 3.0 | | |
| White petrolatum | | | 3.0 | 3.0 | 5.0 | 5.0 | 34.0 | 34.0 |
| Cetyl alcohol | | | 4.0 | 4.0 | 4.0 | 4.0 | | |
| Stearyl Alcohol | 4.0 | 4.0 | 4.0 | 4.0 | 4.0 | 4.0 | 5.0 | 5.0 |
| Glyceryl | | | 5.0 | 5.0 | 3.0 | 3.0 | 5.0 | 5.0 |
| Monosterate Type | | | | | | | | |
| II | | | | | | | | |
| Paraffin, Wax | | | | | | | 3.0 | 3.0 |
| PPG-15 Stearyl | | | | | | | 5.0 | 5.0 |
| ether | | | | | | | | |
| Steareth-2 | 4.1 | 4.1 | | | | | | |
| Steareth-20 | 0.1 | 0.1 | | | | | | |
| Span 80 | | | | | 4.0 | 4.0 | | |
| Polyoxy-40- | | | 4.0 | 4.0 | | | | |
| stearate | | | | | | | | |
| Polysorbate 80 | | | | | 4.0 | 4.0 | | |
| PEG-100 Stearate | 7.0 | 7.0 | | | | | | |
| Cetomacrogol | | | | | | | 5.0 | 5.0 |
| 1000 | | | | | | | | |
| Sepineo P600 | | | | | 3.0 | 3.0 | | |
| Sorbitol Solution | | | 15.0 | 15.0 | | | | |
| 70% | | | | | | | | |
| Butylated | 0.10 | 0.10 | 0.10 | 0.10 | 0.10 | 0.10 | 0.10 | 0.10 |
| hydroxytoluene | | | | | | | | |
| Sorbic acid | 0.10 | 0.10 | 0.10 | 0.10 | 0.10 | 0.10 | 0.10 | 0.10 |
| Methyl Paraben | 0.18 | 0.18 | 0.18 | 0.18 | 0.18 | 0.18 | 0.18 | 0.18 |
| Propyl paraben | 0.02 | 0.02 | 0.02 | 0.02 | 0.02 | 0.02 | 0.02 | 0.02 |
| Disodium Edetate | 0.05 | 0.05 | 0.05 | 0.05 | 0.05 | 0.05 | 0.05 | 0.05 |
| Trolamine (25% | 1.3 | 4.0 | 1.3 | 4.0 | 1.3 | 4.0 | 1.3 | 4.0 |
| w/w Aqueous | | | | | | | | |
| dilution) | | | | | | | | |
| Total | 100.0 | 100.0 | 100.0 | 100.0 | 100.0 | 100.0 | 100.0 | 100.0 |
| pH (To) | 5.75 | 5.75 | 5.6 | 5.60 | 5.91 | 5.91 | 4.53 | 4.53 |

### Example 6. Effects of GPH Adjusting Agents

The effects of pH adjusting agents on the pH value of formulation were evaluated. Formulations containing Compound A mesylate salt (Form B) were prepared with various pH adjusting agent. The formulations were stored at room temperature for up to two months, and the pH values of the formulations were measured during storage and are shown in Table 9. The compositions of the formulations tested in this study are as follows:

Formulation No. 1 - no pH modifiers (standalone neutral excipients)

| | Ingredients | Grade | Brand name | Vendor | % w/w | Batch |
|---|---|---|---|---|---|---|
| 1 | Compound A mesylate salt | | | Pyramid Bio | 3.00 | 15.00 |
| | (Form B) | | | | | |
| 2 | Transcutol-HP | NF | Gattefosse | | 10.00 | 50.00 |
| 3 | Medium chain triglycerides | NF | Captex 300 | ABITEC | 10.00 | 50.00 |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 4.00 | 20.00 |
| 8 | Sorbitol | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy Toluene | USP | | Merck | 0.10 | 0.50 |
| 12 | Butylated hydroxy Anisole | USP | | Merck | 0.10 | 0.50 |
| 13 | Disodium Edetate | USP | EDTA | Merck | 0.05 | 0.250 |
| 14 | Purified water | USP | | Pharmacoaper | 37.75 | 188.75 |

| | | |
|---|---|---|
| Total = | 100.00 | 500.00 |

Formulation No. 2 - trolamine 50% aqueous solution

| | Ingredients | Grade | Brand name | Vendor | % w/w | Batch |
|---|---|---|---|---|---|---|
| 1 | Compound A mesylate | | | Pyramid Bio | 3.00 | 15.00 |
| | salt (Form B) | | | | | |
| 2 | Transcutol-HP | NF | Gattefosse | | 10.00 | 50.00 |
| 3 | Medium chain | NF | Captex 300 | ABITEC | 10.00 | 50.00 |
| | triglycerides | | | | | |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 4.00 | 20.00 |
| 8 | Sorbitol | USP | Neosorb | Roquette | 15.00 | 75.00 |
| 9 | white petrolatum | USP | White protopet | Sonneborn | 3.00 | 15.00 |
| 10 | Polyoxy1-40 stearate | NF | Myrj S-40 | Croda | 4.00 | 20.00 |
| 11 | Butylated hydroxy | USP | | Merck | 0.10 | 0.50 |
| | Toluene | | | | | |
| 12 | Butylated hydroxy | USP | | Merck | 0.10 | 0.50 |
| | Anisole | | | | | |
| 13 | Disodium Edetate | USP | EDTA | Merck | 0.05 | 0.250 |
| 14 | Triethanolamine | USP/Phr.Eu | Trolamine | Merck | 4.00 | 20.000 |
| | (50% Aq. Sol) | | | | | |
| 15 | Purified water | USP | | Pharmaco aper | 33.75 | 168.75 |
| | | | | Total = | 100.00 | 500.00 |

Formulation No. 3 - citrate buffer 0.01M (pH 4.6)

| | | Grade | Brand name | Vendor | % w/w | Batch |
|---|---|---|---|---|---|---|
| 1 | Compound A mesylate salt | | | Pyramid Bio | 3.00 | 15.00 |
| | (Form B) | | | | | |
| 2 | Transcutol HP | NF | | GATTOFFOSSE | 10.00 | 50.00 |
| 3 | Medium chain triglycerides | NF | Captex 300 | ABITEC | 10.00 | 50.00 |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 1.00 | 5.00 |
| 8 | Sorbitol solution 70% | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy Toluene | USP | | Merck | 0.10 | 0.50 |
| 12 | Butylated hydroxy Anisole | USP | | Merck | 0.10 | 0.50 |
| 13 | Disodium Edetate | USP | EDTA | Merck | 0.05 | 0.250 |
| 14 | Citricacid monohydrate | USP | — | Merck | 4.00 | 20.00 |
| 15 | Trisodium citrate dihydrate | USP | — | Merck | | |
| 16 | Purified water | USP | | Pharmaco aper | 36.75 | 183.75 |
| | | | | Total = | 100.00 | 500.00 |

Formulation No. 4 - phosphate buffer 0.01M (pH 5.5)

| | Ingredients | Grade | Brand name | Vendor | % w/w | Batch |
|---|---|---|---|---|---|---|
| 1 | Compound A mesylate | | | Pyramid Bio | 3.00 | 15.00 |
| | salt (Form B) | | | | | |
| 2 | Transcutol HP | NF | | GATTOFFOSSE | 10.00 | 50.00 |
| 3 | Medium chain | NF | Captex 300 | ABITEC | 10.00 | 50.00 |
| | triglycerides | | | | | |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 1.00 | 5.00 |
| 8 | Sorbitol solution 70% | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy | USP | | Merck | 0.10 | 0.50 |
| | Toluene | | | | | |
| 12 | Butylated hydroxy | USP | | Merck | 0.10 | 0.50 |
| | Anisole | | | | | |
| 13 | Disodium Edetate | USP | EDTA | Merck | 0.05 | 0.250 |
| 14 | Phosphate Buffer pH 4.0 | USP | — | Merck | 4.00 | 20.00 |
| | (10X) | | | | | |
| 16 | Purified water | USP | | Pharmaco aper | 36.75 | 183.75 |
| | | | | Total = | 100.00 | 500.00 |

Formulation No. 5 - trolamine 50% ag. solution and citrate buffer 0.01M (pH 4.6)

| | Ingredients | Grade | Brand name | Vendor | % w/w | Batch |
|---|---|---|---|---|---|---|
| 1 | Compound A mesylate salt | | | Pyramid Bio | 3.00 | 15.00 |
| | (Form B) | | | | | |
| 2 | Transcutol HP | NF | | GATTOFFOSSE | 10.00 | 50.00 |
| 3 | Medium chain triglycerides | NF | Captex 300 | ABITEC | 10.00 | 50.00 |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 1.00 | 5.00 |
| 8 | Sorbitol solution 70% | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy Toluene | USP | | Merck | 0.10 | 0.50 |
| 12 | Butylated hydroxy Anisole | USP | | Merck | 0.10 | 0.50 |
| 13 | Disodium Edetate | USP | EDTA | Merck | 0.05 | 0.250 |
| 14 | Triethanolamine (50% diluted) | USP | Trolamine | Merck | 2.00 | 10.00 |
| 15 | Citric acid monohydrate | USP | — | Merck | 2.00 | 10.00 |
| 16 | Trisodium citrate dihydrate | USP | — | Merck | | |
| 17 | Purified water | USP | | Pharmaco aper | 36.75 | 183.75 |
| | | | | Total = | 100.00 | 500.00 |

Formulation No. 6 - trolamine 50% aq. solution 2.5% w/w

| | Ingredients | Grade | Brand name | Vendor | % w/w | Batch |
|---|---|---|---|---|---|---|
| 1 | Compound A mesylate salt | | | Pyramid Bio | 3.00 | 15.00 |
| | (Form B) | | | | | |
| 2 | Transcutol-HP | NF | Gattefosse | | 10.00 | 50.00 |
| 3 | Medium chain | NF | Captex 300 | ABITEC | 10.00 | 50.00 |
| | triglycerides | | | | | |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 4.00 | 20.00 |
| 8 | Sorbitol | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy | USP | | Merck | 0.10 | 0.50 |
| | Toluene | | | | | |
| 12 | Butylated hydroxy Anisole | USP | | Merck | 0.10 | 0.50 |
| 13 | Disodium Edetate | USP | EDTA | Merck | 0.05 | 0.250 |
| 14 | Triethanolamine (50% Aq. | USP/Phr.Eu | Trolamine | Merck | 2.50 | 12.500 |
| | Sol) | | | | | |
| 15 | Purified water | USP | | Pharmaco aper | 35.25 | 176.25 |
| | | | | Total = | 100.00 | 500.00 |

Formulation No. 7 - trolamine 25% ag. solution

| | Ingredients | Grade | Brand name | Vendor | %w/w | Batch |
|---|---|---|---|---|---|---|
| 1 | Compound A mesylate salt | | | Pyramid Bio | 3.00 | 15.00 |
| | (Form B) | | | | | |
| 2 | Transcutol-HP | NF | Gattefosse | | 10.00 | 50.00 |
| 3 | Medium chain | NF | Captex 300 | ABITEC | 10.00 | 50.00 |
| | triglycerides | | | | | |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 4.00 | 20.00 |
| 8 | Sorbitol | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy | USP | | Merck | 0.10 | 0.50 |
| | Toluene | | | | | |
| 12 | Butylated hydroxy Anisole | USP | | Merck | 0.10 | 0.50 |
| 13 | Disodium Edetate | USP | EDTA | Merck | 0.05 | 0.250 |
| 14 | Triethanolamine (25% Aq. | USP/Phr.Eu | Trolamine | Merck | 4.00 | 20.000 |
| | Sol) | | | | | |
| 15 | Purified water | USP | | Pharmaco aper | 33.75 | 168.75 |
| | | | | Total = | 100.00 | 500.00 |

Formulation No. 8 - tromethamine

| | Ingredients | Grade | Brand name | Vendor | % w/w | Batch |
|---|---|---|---|---|---|---|
| 1 | Compound A | | | Pyramid Bio | 3.00 | 15.00 |
| | mesylate salt (Form | | | | | |
| | B) | | | | | |
| 2 | Transcutol-HP | NF | Gattefosse | | 10.00 | 50.00 |
| 3 | Medium chain | NF | Captex 300 | ABITEC | 10.00 | 50.00 |
| | triglycerides | | | | | |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 4.00 | 20.00 |
| 8 | Sorbitol | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy | USP | | Merck | 0.10 | 0.50 |
| | Toluene | | | | | |
| 12 | Butylated hydroxy | USP | | Merck | 0.10 | 0.50 |
| | Anisole | | | | | |
| 13 | Disodium Edetate | USP | EDTA | Merck | 0.05 | 0.250 |
| 14 | Tromethamine | USP/Phr.Eu | | Merck | 5.00 | 25.000 |
| | (25% Aq. Sol) | | | | | |
| 15 | Purified water | USP | | Pharmaco aper | 32.75 | 163.75 |
| | | | | Total = | 100.00 | 500.00 |

Formulation No. 9 - ethylenediamine

| | Ingredients | Grade | Brand name | Vendor | % w/w | Batch |
|---|---|---|---|---|---|---|
| 1 | Compound A mesylate | | | Pyramid Bio | 3.00 | 15.00 |
| | salt (Form B) | | | | | |
| 2 | Transcutol-HP | NF | Gattefosse | | 10.00 | 50.00 |
| 3 | Medium chain | NF | Captex 300 | ABITEC | 10.00 | 50.00 |
| | triglycerides | | | | | |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 4.00 | 20.00 |
| 8 | Sorbitol | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy | USP | | Merck | 0.10 | 0.50 |
| | Toluene | | | | | |
| 12 | Butylated hydroxy | USP | | Merck | 0.10 | 0.50 |
| | Anisole | | | | | |
| 13 | Disodium Edetate | USP | EDTA | Merck | 0.05 | 0.250 |
| 14 | Ethylenediamine 25% | | | Merck | 2.50 | 12.500 |
| | Aq sol | | | | | |
| 15 | Purified water | USP | | Pharmaco aper | 35.25 | 176.25 |
| | | | | Total = | 100.00 | 500.00 |

Formulation No. 10 - diethanolamine

| | Ingredients | Grade | Brand name | Vendor | % w/w | Batch |
|---|---|---|---|---|---|---|
| 1 | Compound A mesylate salt | | | Pyramid Bio | 3.00 | 15.00 |
| | (Form B) | | | | | |
| 2 | Transcutol-HP | NF | Gattefosse | | 10.00 | 50.00 |
| 3 | Medium chain | NF | Captex 300 | ABITEC | 10.00 | 50.00 |
| | triglycerides | | | | | |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 4.00 | 20.00 |
| 8 | Sorbitol | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy | USP | | Merck | 0.10 | 0.50 |
| | Toluene | | | | | |
| 12 | Butylated hydroxy | USP | | Merck | 0.10 | 0.50 |
| | Anisole | | | | | |
| 13 | Disodium Edetate | USP | EDTA | Merck | 0.05 | 0.250 |
| 14 | Diethanolamine | | | Merck | 2.50 | 12.500 |
| | 25% Aq sol | | | | | |
| 15 | Purified water | USP | | Pharmaco aper | 35.25 | 176.25 |
| | | | | Total = | 100.00 | 500.00 |

Formulation No. 11 - tromethamine 3.3% w/w

| | Ingredients | Grade | Brand name | Vendor | % w/w | Batch |
|---|---|---|---|---|---|---|
| 1 | Compound A | | | Pyramid Bio | 3.00 | 15.00 |
| | mesylate salt (Form | | | | | |
| | B) | | | | | |
| 2 | Transcutol-HP | NF | Gattefosse | | 10.00 | 50.00 |
| 3 | Medium chain | NF | Captex 300 | ABITEC | 10.00 | 50.00 |
| | triglycerides | | | | | |
| 4 | GMS-II | NF | Kolliwax GMS-II | BASF | 5.00 | 25.00 |
| 5 | Cetyl alcohol | NF | Crodacol C90 | CRODA | 4.00 | 20.00 |
| 6 | Stearyl alcohol | NF | Crodacol S95 | Croda | 4.00 | 20.00 |
| 7 | Benzyl alcohol | NF | — | Merck | 4.00 | 20.00 |
| 8 | Sorbitol | USP | Neosorb | Roquette | 15.00 | 75.00 |
| 9 | white petrolatum | USP | White protopet | Sonneborn | 3.00 | 15.00 |
| 10 | Polyoxy1-40stearate | NF | Myrj S-40 | Croda | 4.00 | 20.00 |
| 11 | Butylated hydroxy | USP | | Merck | 0.10 | 0.50 |
| | Toluene | | | | | |
| 12 | Butylated hydroxy | USP | | Merck | 0.10 | 0.50 |
| | Anisole | | | | | |
| 13 | Disodium Edetate | USP | EDTA | Merck | 0.05 | 0.250 |
| 14 | Tromethamine | USP/Phr.Eu | | Merck | 3.3 | 25.000 |
| | (25% Aq. Sol) | | | | | |
| 15 | Purified water | USP | | Pharmaco aper | 34.45 | 172.25 |
| | | | | Total = | 100.00 | 500.00 |

NF = National formulary; Ph Eur = European Pharmacopeia; USP = United States Pharmacopeia

The formulations were stored at room temperature for up to two months, and the pH values of the formulations were measured during the storage and are shown in Table 9.

TABLE 9

| | | % w/w pH adjusting agents | | | | | | | pH Measurements | | | | | |
|---|---|---|---|---|---|---|---|---|---|---|---|---|---|---|
| No. | Product | B¹ | C² | D³ | E⁴ | F⁵ | G⁶ | H⁷ | Day 1 | Day 3 | Day 7 | Day 14 | Day 20 | Day 60 |
| 1 | Compound A | with no pH modifiers | | | | | | | 1.89 | 1.89 | 1.89 | 1.92 | 1.96 | |
| | mesylate salt | (standalone Neutral excipients) | | | | | | | | | | | | |
| | Cream, 3.0% | | | | | | | | | | | | | |
| 2 | Compound A | 4.0 | | | | | | | 8.15 | 8.15 | 8.11 | 7.84 | 8.34 | |
| | mesylate salt | | | | | | | | | | | | | |
| | Cream, 3.0% | | | | | | | | | | | | | |
| 3 | Compound A | | | | 4.0 | | | | 2.16 | 2.08 | 2.01 | 2.00 | 1.98 | |
| | mesylate salt | | | | | | | | | | | | | |
| | Cream, 3.0% | | | | | | | | | | | | | |
| 4 | Compound A | | | 4.0 | | | | | 2.11 | 1.98 | 1.99 | 1.98 | 2.01 | |
| | mesylate salt | | | | | | | | | | | | | |
| | Cream, 3.0% | | | | | | | | | | | | | |
| 5 | Compound A | 2.0 | | | 2.0 | | | | 8.14 | 8.14 | 8.10 | 8.11 | 8.06 | 8.09 |
| | mesylate salt | | | | | | | | | | | | | |
| | Cream, 3.0% | | | | | | | | | | | | | |
| 6 | Compound A | 2.5 | | | | | | | 7.15 | 7.01 | 6.99 | 7.01 | 7.10 | 6.88 |
| | mesylate salt | | | | | | | | | | | | | |
| | Cream, 3.0% | | | | | | | | | | | | | |
| 7 | Compound A | | 4.0 | | | | | | 6.02 | 5.92 | 5.98 | 6.02 | 6.13 | 5.9 |
| | mesylate salt | | | | | | | | | | | | | |
| | Cream, 3.0% | | | | | | | | | | | | | |
| 8 | Compound A | | | | | 5.0 | | | 7.82 | 7.76 | 7.65 | 7.76 | 7.82 | |
| | mesylate salt | | | | | | | | | | | | | |
| | Cream, 3.0% | | | | | | | | | | | | | |
| 9 | Compound A | | | | | | 2.5 | | 2.68 | 2.54 | 2.5 | 2.45 | 2.18 | |
| | mesylate salt | | | | | | | | | | | | | |
| | Cream, 3.0% | | | | | | | | | | | | | |
| 10 | Compound A | | | | | | | 2.5 | 2.42 | 2.32 | 2.34 | 2.12 | 2.35 | |
| | mesylate salt | | | | | | | | | | | | | |
| | Cream, 3.0% | | | | | | | | | | | | | |
| 11 | Compound A | | | | | 3.3 | | | 6.05 | 6.12 | 6.45 | 6.63 | 7.52 | |
| | mesylate salt | | | | | | | | | | | | | |
| | Cream, 3.0% | | | | | | | | | | | | | |

¹B corresponds to trolamine, 50% w/w; ²C corresponds to trolamine 25% w/w; ³D corresponds to phosphate buffer 0.01M (pH 5.5); ⁴E corresponds to citrate buffer 0.01M (pH 4.6); ⁵F corresponds totromethamine 25% w/w; ⁶G corresponds to ethylenediamine 25% w/w; and ⁷H corresponds todiethanolamine 25% w/w.

The results shown in Table 9 demonstrate that the citrate and phosphate buffers were unable to maintain the pH of the cream at an acceptable level of pH˜5.5. The weak organic base pH adjusting agents performed well. The amine pH adjusting agents tested were able to increase the pH of the compositions and maintain the pH over a period of time. In particular, the tertiary amines were particularly effective. Trolamine diluted at 25% o was able to sufficiently maintain the pH of the cream formulation prototype.

The 3% w/w pH optimized formulations which were prepared according to Table 8 were put on stability for 3 months at 25° C./60% RH and 40° C./75% RH. The pH and assay of the compositions were measured at 0, 1, 2 and 3 months. The results are presented in Table 10.

TABLE 10
pH and assay Stability Data

| | Test | pH at Test Conditions | | Assay (% L.C.) | |
|---|---|---|---|---|---|
| | Periods | 25° C., | 40° C., | 25° C., | 40° C., |
| Formulation | (months) | 60% RH | 75% RH | 60% RH | 75% RH |
| F1 Placebo | 0 | 8.0 | | NA | |
| | 1 | 8.47 | 8.39 | | |
| | 2 | 8.37 | 8.30 | | |
| | 3 | 8.36 | 8.32 | | |
| F1, 3% pH | 0 | 5.56 | | 103.9 | |
| opt Cream | 1 | 5.52 | 5.63 | 100 | 99.9 |
| | 2 | 5.57 | 5.51 | 103.3 | 103.1 |
| | 3 | 5.49 | 5.60 | 102.0 | 101.5 |
| F6, pH opt | 0 | 7.60 | | NA | |
| Placebo | 1 | 8.42 | 8.32 | | |
| | 2 | 8.31 | 8.20 | | |
| | 3 | 8.36 | 8.08 | | |
| F6, 3% pH | 0 | 6.74 | | 102.3 | |
| opt Cream | 1 | 6.05 | 6.13 | 98.8 | 99.0 |
| | 2 | 6.11 | 6.01 | 101.7 | 101.6 |
| | 3 | 6.17 | 6.01 | 101.2 | 100.5 |
| F8, pH opt | 0 | 7.40 | | NA | |
| Placebo | 1 | 8.39 | 8.41 | | |
| | 2 | 8.07 | 8.25 | | |
| | 3 | 8.23 | 8.08 | | |
| F8, 3% pH | 0 | 6.04 | | 104.4 | |
| opt Cream | 1 | 5.90 | 5.94 | 100.6 | 100.8 |
| | 2 | 6.01 | 5.84 | 104.1 | 105.9 |
| | 3 | 5.87 | 5.86 | 102.5 | 102.1 |
| F9, pH opt | 0 | 8.10 | | NA | |
| Placebo | 1 | 7.90 | 8.34 | | |
| | 2 | 8.38 | 8.24 | | |
| | 3 | 8.37 | 8.11 | | |
| F9, 3% pH | 0 | 6.03 | | 106.0 | |
| Cream | 1 | 5.94 | 5.56 | 107.5 | 102.0 |
| | 2 | 6.03 | 5.94 | 104.4 | 69.8 |
| | 3 | 5.96 | 5.88 | 103.5 | 64.6 |

pH titration with trolamine was shown to stabilize the pH of the prototype 300 pH optimised formulations according to Table 8 over the 3 month period.

It was noted that formulation F9 3% pH optimized presented with heterogeneous distribution of oil globules. This is thought to have contributed to the low assay value observed for the F9 3% pH optimized formulation at 2 and 3 months 40° C./75% RH.

It was also found that particular weak organic base pH adjusting agents (e.g. tertiary amines such as triethanolamine) were not only able to adjust pH values but were also able to improve the stability of certain compositions and prevent phase separation. For example, formulation numbers 3 and 4 from Table 9, which are compositions using non-organic base pH adjusting agents, i.e. phosphate or citrate buffers (labelled as D or E in Table 9), were unable to buffer pH. Furthermore, the compositions containing these non-organic base pH adjusting agents were also found to be physically unstable and formed visible clumps and agglomerates. In contrast, compositions employing particular weak organic base pH adjusting agents (e.g. tertiary amines such as triethanolamine) were not only able to adjust pH values but were also able to improve the stability of certain compositions. Without being bound to any theory, organic bases with hydroxyl groups may aid in the solubilization of Compound A and due to a level of miscibility with an aqueous phase, may also prevent phase separation for an emulsion-based composition such as a cream.

### Example 7. Further Exemplary Compositions

Exemplary formulations of the present disclosure containing 1% w/w of Compound A mesylate salt (Form B) were prepared as follows.

### Preparation of 1% Formulation, F1, 1% pH Opt.

Preparation of aqueous phase: In a 1 liter stainless steel container equipped with an IKA overhead stirrer is added Steareth-20 (0.1 g), PEG-100 stearate (7 g), sorbic acid (0.1 g), methyl paraben (0.18 g) and disodium edetate (0.05 g) and water (67.05 g). Heated to 65-70° C. using a water bath. Mix until all the solids are dissolved. Add Compound A mesylate salt (1 g), heat and stir until dissolved.

Preparation of oil phase: In a 1 liter stainless steel container equipped with an IKA overhead stirrer is added Dimethicone (5 g), Stearyl alcohol (4 g), Steareth-2 (4.1 g), Medium Chain Triglycerides (10 g), Butylated hydroxy Toluene (0.1 g) and Propyl paraben (0.02 g). The reaction mixture is heated to 65-70° C. with stirring until all the solids have dissolved.

Add the aqueous phase mixture to the oil phase mixture and stir for 15-20 min using a Silverson homogenizer. Cool the reaction mixture to below 30° C. and add Trolamine (25% w/w, 0.75 g) and stir for additional 5 min. Check pH of the product to make sure it is 5±1.

### Preparation of 1% Formulation, F6, 1% pH Opt.

Preparation of aqueous phase: In a 1 liter stainless steel container equipped with an IKA overhead stirrer is added Water (42.25 g), polyoxy-40-stearate (4 g), sorbitol solution (15 g), sorbic acid (0.1 g), methyl paraben (0.18 g) and disodium edetate (0.05 g). Set up water bath at 65-70° C. Using IKA overhead mixer (propeller) mix all the contents in the water bath until all solids are dissolved. When the reaction mixture reaches 45-50° C., add Compound A mesylate salt (1 g), heat and stir until dissolved.

Preparation of oil phase: In a 1 liter stainless steel container equipped with an IKA overhead stirrer is added add medium chain triglycerides (10 g), white petrolatum (3 g), cetyl alcohol (4 g), stearyl alcohol (4 g), glyceryl monostearate Type II (5 g), Butylated hydroxy Toluene (0.1 g) and Propyl paraben (0.02 g). The reaction mixture is heated to 65-70° C. with stirring until all the solids have dissolved.

Add the aqueous phase mixture to the oil phase mixture and stir at 3000-3500 rpm for 20 min using a Silverson homogenizer. Cool the reaction mixture to below 30° C. and add Transcutol HP (10 g). Then add Trolamine (25% w/w, 1.3 g) and stir for additional 5 min. Check pH of the product to make sure it is 5±1.

### Preparation of 1% Formulation, F8, 1% pH Opt.

Preparation of aqueous phase: In a 1 liter stainless steel container equipped with an IKA overhead stirrer is added Water (44.25 g), polysorbate 80 (4 g), sorbic acid (0.1 g), methyl paraben (0.18 g), and disodium edetate (0.05 g). Heated to 65-70° C. using a water bath. Mix until all the solids are dissolved. Add Compound A mesylate salt (1 g), heat and stir until dissolved.

Preparation of oil phase: In a 1 liter stainless steel container equipped with an IKA overhead stirrer is added oleyl alcohol (3 g), octyldodecanol (5 g), cyclomethicone (5 g), dimethicone (3 g), white petrolatum (5 g), cetyl alcohol (4 g), stearyl alcohol (4 g), glyceryl monostearate Type II (3 g), span 80 (4 g), Butylated hydroxy Toluene (0.1 g), and Propyl paraben (0.02 g). The reaction mixture is heated to 65-70° C. with stirring until all the solids have dissolved.

Add the aqueous phase mixture to the oil phase mixture and stir for 15-20 min using a Silverson homogenizer. Cool the reaction mixture to below 30° C. and add Transcutol HP (10 g, preheated to 40° C.). Cool the reaction mixture and add Sepino P 600 (3 g) and mix well. When the reaction mixture reaches 35° C., add Trolamine (25% w/w, 1.3 g) and stir for additional 5 minutes. Check pH of the product to make sure it is 5±1.

### Preparation of 1% Formulation, F9, 1% pH Opt.

Preparation of aqueous phase: In a 1 liter stainless steel container equipped with an IKA overhead stirrer is added Water (25.25 g), cetomacrogel 1000 (5 g), sorbic acid (0.1 g), methyl paraben (0.18 g) and disodium edetate (0.05 g). Heated to 65-70° C. using a water bath. Mix until all the solids are dissolved. Add Compound A mesylate salt (1 g), heat and stir until dissolved.

Preparation of oil phase: In a 1-liter stainless steel container equipped with an IKA overhead stirrer is added cyclomethicone (5 g), white petrolatum (34 g), stearyl alcohol (5 g), glyceryl monostearate (5 g), paraffin wax (3 g), PPG-15 stearyl ether (5 g), Butylated hydroxy Toluene (0.1 g) and Propyl paraben (0.02 g). The reaction mixture is heated to 65-70° C. with stirring until all the solids have dissolved.

Add the aqueous phase mixture to the oil phase mixture and stir for 15-20 min using a Silverson homogenizer. Cool the reaction mixture to below 30° C. and add Transcutol HP (10 g, preheated to 40° C.). Cool the reaction mixture. When the reaction mixture reaches 35° C., add Trolamine (25% w/w, 1.3 g) and stir for additional 5 min. Check pH of the product to make sure it is 5±1.

### Preparation of 1% Formulation, F10. 1% pH Opt.

Preparation of aqueous phase: In a glass beaker container equipped with an IKA overhead stirrer is added Transcutol HP (10 g) and Compound A mesylate salt (1 g). Heated to 45-50° C. using a water bath. Mix until all the solids are dissolved.

Preparation of oil phase: In a glass beaker equipped with an IKA overhead stirrer is added dimethicone (5 g), white petrolatum (70.5 g), Glyceryl monostearate Type II (3.9 g), White beeswax (3 g), Glycerin 5 g), and Butylated hydoxy Toluene (0.1 g). The reaction mixture is heated to 60-65° C. with stirring until all the solids have dissolved.

Add the oil phase mixture to the aqueous phase mixture and stir for 15-20 min using a Silverson homogenizer. Cool the reaction mixture to below 30° C. When the reaction mixture reaches 35° C., add Trolamine (25% w/w, 1.3 g) and stir for additional 5 min. Check pH of the product to make sure it is 5±1.

### Preparation of 0.1% Formulation, F8, 0.1% pH Optimised

Preparation of aqueous phase: In a 1 liter stainless steel container equipped with an IKA overhead stirrer is added Water (46.44 g), polysorbate 80 (8 g), methyl paraben (0.18 g), and disodium edetate (0.05 g). Heated to 65-70° C. using a water bath. Mix all the solids at 200-500 rpm until everything is dissolved. Reduce the temperature to 60° C.±5° C. and add Transcutol HP (5 g). Cool the reaction mixture to 45° C.±5° C. and add Compound A mesylate salt (0.12 g), heat and stir until dissolved.

Preparation of oil phase: In a 1 liter stainless steel container equipped with an IKA overhead stirrer is added octyldodecanol (3 g), oleyl alcohol (3 g), cyclomethicone (3 g), white petrolatum (15 g), cetyl alcohol (3 g), stearyl alcohol (3 g), glyceryl monostearate Type II (7 g), span 80 (3 g), Butylated hydroxy Toluene (0.1 g), sorbic acid (0.1 g), and Propyl paraben (0.02 g). The reaction mixture is heated to 65-70° C. with stirring at 200-500 RPM until all the solids have dissolved and a clear solution is obtained.

Add the aqueous phase mixture to the oil phase mixture and stir for 25-30 min using overhead stirrer. Cool the reaction mixture to 25° C.±5° C. When the reaction mixture reaches 35° C., add Trolamine (25% w/w, 0.1 g) and stir for additional 5 min. Check pH of the product to make sure it is 5±1.

### Preparation of 0.03% Formulation, F8, 0.03% pH Optimised

Preparation of aqueous phase: In a 1 liter stainless steel container equipped with an IKA overhead stirrer is added Water (46.5 g), polysorbate 80 (8 g), methyl paraben (0.18 g), and disodium edetate (0.05 g). Heated to 65-70° C. using a water bath. Mix all the solids at 200-500 rpm until everything is dissolved. Reduce the temperature to 60° C.±5° C. and add Transcutol HP (5 g). Cool the reaction mixture to 45° C.±5° C. and add Compound A mesylate salt (0.036 g), heat and stir until dissolved.

Preparation of oil phase: In a 1 liter stainless steel container equipped with an IKA overhead stirrer is added octyldodecanol (3 g), oleyl alcohol (3 g), cyclomethicone (3 g), white petrolatum (15 g), cetyl alcohol (3 g), stearyl alcohol (3 g), glyceryl monostearate Type II (7 g), span 80 (3 g), Butylated hydroxy Toluene (0.1 g), sorbic acid (0.1 g), and Propyl paraben (0.02 g). The reaction mixture is heated to 65-70° C. with stirring at 200-500 RPM until all the solids have dissolved and a clear solution is obtained.

Add the aqueous phase mixture to the oil phase mixture and stir for 25-30 min using overhead stirrer. Cool the reaction mixture to 25° C.±5° C. When the reaction mixture reaches 35° C., add Trolamine (25% w/w, 0.1 g) and stir for additional 5 min. Check pH of the product to make sure it is 5±1.

### Preparation of 0.01% Formulation, F8, 0.01% pH Optimised

Preparation of aqueous phase: In a 1 liter stainless steel container equipped with an IKA overhead stirrer is added Water (41.86 g), polysorbate 80 (8 g), methyl paraben (0.18 g), and disodium edetate (0.05 g). Heated to 65-70° C. using a water bath. Mix all the solids at 200-500 rpm until everything is dissolved. Reduce the temperature to 60° C.±5° C. and add Transcutol HP (5 g). Cool the reaction mixture to 45° C.±5° C. and add Compound A mesylate salt (0.012 g), heat and stir until dissolved.

Preparation of oil phase: In a 1 liter stainless steel container equipped with an IKA overhead stirrer is added octyldodecanol (3 g), oleyl alcohol (3 g), cyclomethicone (3 g), white petrolatum (15 g), cetyl alcohol (3 g), stearyl alcohol (3 g), glyceryl monostearate Type II (7 g), span 80 (3 g), Butylated hydroxy Toluene (0.1 g), sorbic acid (0.1 g), and Propyl paraben (0.02 g). The reaction mixture is heated to 65-70° C. with stirring at 200-500 RPM until all the solids have dissolved and a clear solution is obtained.

Add the aqueous phase mixture to the oil phase mixture and stir for 25-30 min using overhead stirrer. Cool the reaction mixture to 25° C.±5° C. When the reaction mixture reaches 35° C., add Trolamine (25% w/w, 0.1 g) and stir for additional 5 min. Check pH of the product to make sure it is 5±1.

### Example 8. Irritancy Levels of Exemplary Formulations

The irritancy levels of certain exemplary formulations were evaluated with the MatTek EpiDerm (EPI-200) in vitro skin model. The EpiDerm model consists of normal, human derived epidermal keratinocytes which have been cultured on specially prepared cell culture inserts using serum free medium to form a stratified, highly differentiated model of human skin. As a measure of dermal irritation, the cell viability and IL-1α release of EpiDerm cultures are monitored over time after topical exposure to the product. Cell viability is monitored using the MTT assay, a colorimetric assay system that measures the reduction of a yellow tetrazolium component (MTT) into an insoluble purple formazan product by the mitochondria of viable cells. The amount of color produced is directly proportional to the number of viable cells. ET-50 refers to the estimated time to reduce tissue viability by 50%.

EpiDerm Model. Upon receipt, EpiDerm cultures (EPI-200) were placed into media (EPI-100-ASY; 0.9 mL/well in a 6-well plate) and equilibrated at 37° C./5% C02, overnight. The following exemplary formulations described in Table 11 were tested.

Composition details are described in Example 5, Tables 5 and 6. For the pH optimised F1, F6, F8 and F9 formulations, composition details are described in Example 5, Table 8.

TABLE 11

| # | Description | Timepoints |
|---|---|---|
| 1 | Untreated (negative) | 3, 7, 24 hrs |
| | Control | |
| 2 | 1% Tx-100 (positive) | 3, 7, 24 hrs |
| | Control for irritation | |
| 3 | Tazorotene 0.1% | 3, 7, 24 hrs |
| 4 | Compound A mesylate | 3, 7, 24 hrs |
| | salt in water | |
| 5 | F1, 1% pH opt | 3, 7, 24 hrs |
| 6 | F1, 3% pH opt | 3, 7, 24 hrs |
| 7 | F2,1% | 3, 7, 24 hrs |
| 8 | F2, 3% | 3, 7, 24 hrs |
| 9 | F3, 1% | 3, 7, 24 hrs |
| 10 | F3, 3% | 3, 7, 24 hrs |
| 11 | F4, 1% | 3, 7, 24 hrs |
| 12 | F4, 3% | 3, 7, 24 hrs |
| 13 | F5, 1% | 3, 7, 24 hrs |
| 14 | F5, 3% | 3, 7, 24 hrs |
| 15 | F6, 1% pH opt | 3, 7, 24 hrs |
| 16 | F6, 3% pH opt | 3, 7, 24 hrs |
| 17 | F7, 1% | 3, 7, 24 hrs |
| 18 | F7, 3% | 3, 7, 24 hrs |
| 19 | F8, 1% pH opt | 3, 7, 24 hrs |
| 20 | F8, 3% pH opt | 3, 7, 24 hrs |
| 21 | F9, 1% pH opt | 3, 7, 24 hrs |
| 22 | F9, 3% pH opt | 3, 7, 24 hrs |
| 23 | F10, 1% | 3, 7, 24 hrs |
| 24 | F10, 3% | 3, 7, 24 hrs |

### Methods

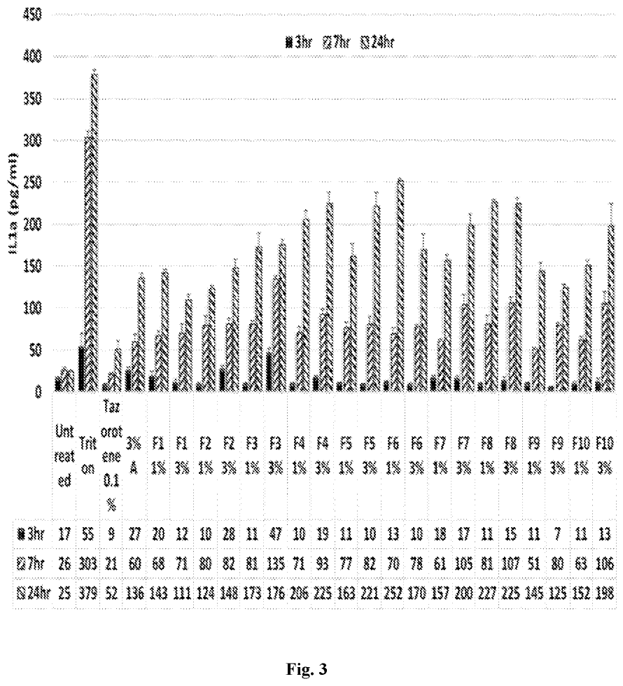

The assay was conducted according to the following procedure. Briefly, 100 μL of the test articles were applied topically to cultures (in triplicates). Tissues were exposed to the Test Articles for 3, 7, or 24 hours at 37° C./5% CO₂, as recommended by the supplier. At the end of the exposure times, media was collected and stored at −20° C., until further use. IL-1α concentration was assessed by ELISA (Human IL-1α Quantikine® Elisa kit, R&D Systems, DLA50). At the end of the incubation period, test articles were carefully washed off with DPBS. Cultures were transferred to a 24-well plate containing 0.3 mL/well MTT solution and incubated for 3 hours at 37° C./5% CO₂. After the MTT incubation, cultures were immersed into 2 mL/well isopropanol in a 24-well plate. The plate was sealed with parafilm to minimize evaporation and incubated at room temperature, overnight, in the dark. The extract solution was transferred to a 96-well plate (0.2 ml/well). Each triplicate was assessed in duplicates for a total of 6 replicates. Optical density was measured at 570 nm (background at 650 nm was subtracted), using isopropanol as a blank. Cell viability was calculated: 100×[OD₍ₛₐₘₚₗₑ₎/OD_{(neg control)}]. The ET-50 was calculated by interpolation of the time at which the % viability has dropped to 50%. According to the Mattek EpiDerm protocol, the irritancy potential is directly correlated with the effective time (ET-50) values as shown in Table 12 below. A summary of the ET-50 data and irritancy classification for the exemplary formulations is shown in FIG. 2. Further, a summary of the IL-1α data is shown FIG. 3.

The MTT assay data shows that all the formulations containing 1% of Compound A mesylate salt were classified as very mild irritants. The formulations containing 3% of Compound A mesylate salt were classified as either very mild or moderate to mild irritants. The best prototypes based on the skin irritancy studies (MTT assay and IL-1α expression) are F1, F6, F8 and F9.

TABLE 12

| ET-50 (hrs) | Expected In Vivo Irritancy |
|---|---|
| >24 | Non-Irritating |
| 12-24 | Very Mild |
| 4-12 | Moderate-to-Mild |
| 0.5-4 | Moderate |
| <0.5 | Strong/Severe, Possible Corrosive |

### Example 9: Topical Penetration Profile of Exemplary Formulations

### Human Skin Preparation.

Full thickness human abdominal skin, cryopreserved at −80° C., was thawed to room temperature, cut into 1.5 cm² section and mounted, strateum corneum side up, onto diffusion cell. Transepidermal water loss (TEWL) value should be 5-25 g/m² h.

### Diffusion Cell Chamber

PermeGear Collector FC33, Version 3.1). Diffusion cells placed in heated support to maintain temperature at 32° C.±2° C. Diffusion cells connected to multi-channel peristaltic pumps and outlet directly connected to tared 20 mL scintillation vials. The flow rate is approximately 1.8 mL/h, and the cells are allowed to equilibrate for approximately 30 minutes.

### Test Protocol.

Test sample is uniformly applied onto the stratum corneum surface using positive displacement pipet, set to deliver 10 μL sample in open donor chamber (skin not occluded). Fractions collected at specified time points (up to 24 hours). Scintillation vials weighed before analysis. Skin sample cleaned with cotton swabs (one dry and one wet with hexane-acetonitrile 1:9). Skin sample tape stripped three times for analysis. Skin sample was placed, dermis side down, on aluminum foil and heated in 60° C. oven for 2.5 minutes. Dermis was separated from epidermis using tweezers and placed in tared vials. Water-acetonitrile (1:1) was added to prepare 150 μl/mg sample (epidermis) and 9 μl/mg sample (dermis). Samples homogenized with up to additional 500 μL water-acetonitrile (1:1) and then centrifuged at 10000 RPM for 5 minutes at 5° C.

### LC-MS Sample Analysis.

All receptor solution samples at different time points and skin samples for each cell after the final time point were analyzed for Compound A content using LC-MS on a Waters Xevo G2-XS QT of using a Waters XBridge C18 3.5 um 3×50 mm column. Mobile phases A and B were 0.1% formic in water and 0.1% formic acid in acetonitrile respectively. Flow rate was set at 0.200 mL/min with a gradient from 20% Mobile Phase B to 80% Mobile Phase B. The analyte eluted at 3.19 minutes. The MS was run in positive mode, and the analyte was quantitated using a mass of 383.1704 Da.

In-vitro permeation (IVPT) studies conducted on F1, F6, F8, and F9 (compositions comprising 1% w/w and 3% w/w N-[(1S)-1-(5-fluoropyrimidin-2-yl)ethyl]-3-(5-isopropoxy-1H-pyrazol-3-yl)-3H-imidazo[4,5-b]pyridin-5-amine mesylate, Form B) are described in Table 13. The preparations of the compositions tested are described in Example 5.

TABLE 13
IVPT at 6 and 24 h

| | | | Epidermis + | Media | Epidermis | | Epidermis + | Media |
|---|---|---|---|---|---|---|---|---|
| | Epidermis | Dermis | Dermis (ng), | (ng), | (ng), | Dermis | Dermis (ng), | (ng), |
| Formulation | (ng), 6 h | (ng), 6 h | 6 h | 6 h | 24 h | (ng), 24 h | 24 h | 24 h |
| F1 1% | 1433.3 | 789.0 | 2222.3 | ND | 1065.8 | 1458.0 | 2523.8 | 39.7 |
| F1 3% | | | | | 2234.2 | 1664.0 | 3898.2 | 152.1 |
| F6 1% | 1766.7 | 1145.0 | 2911.7 | 5.9 | 1497.5 | 1452.0 | 2949.5 | 4.8 |
| F6 3% | | | | | 3325.0 | 2506.0 | 5831 | 21.1 |
| F8 1% | 1651.7 | 1614.0 | 3265.7 | ND | 1331.7 | 1348.0 | 2679.7 | 47.9 |
| F8 3% | | | | | 4591.7 | 2335.0 | 6926.7 | 62.7 |
| F9 1% | 1784.2 | 1338.0 | 3122.2 | ND | 2023.3 | 1840.0 | 3863.3 | 140.1 |
| F9 3% | | | | | 2834.2 | 1994.0 | 4828.2 | 185 |

As can be seen from Table 13, the permeation profiles were found to be dose dependent. The results show that the compositions were able to provide significant skin permeation with acceptable levels of compound reaching the tissue layers. The results also showed that formulations containing transcutol (i.e. formulations F6, F8, and F9) are superior to formulations without transcutol (F1) at delivering significant amounts of compound to each of the skin layers, epidermis and dermis, at both time-points tested, 6 and 24 hours, irrespective of the final concentration of compound, i.e. 1 or 3%. Out of the 3 formulations containing transcutol, F6 and F8 provided high levels in the epidermis and dermis skin layers (i.e. the target site) whilst keeping levels in the receptor fluid media low when compared to F9 at both concentrations, i.e. 1 and 3%. When compared to F6, F8 showed the highest amount of compound retained in the skin layers combined (i.e. epidermis plus dermis) after 6 hours at the lowest concentration of 1% as well as at the highest concentration of 3% after 24 hours. The overall release performance, together with other factors, including suitable aesthetic properties over other prototype formulations, made F8 the lead prototype to take forward.

### Example 10: Stability Study of Compositions

Compositions at strengths of 0.03% w/w and 0.1% w/w were made using analogous methods to those described for F8, 0.01% pH optimised in Example 7. These compositions were placed in amber glass jars and were stored at 25° C./60% RH for 3 months. The results are presented in Table 14.

TABLE 14
3 month stability for the 0.03% and 0.1% w/w compositions of Example 7.

| Test | 0.03% w/w cream | 0.1% w/w cream |
|---|---|---|
| Appearance (visual) | White cream free of foreign particles | White cream free of foreign particles |
| Microscopy | Observations Under 10x Objective | Observations Under 10x Objective |
| | (white light and polarized light): | (white light and polarized light): |
| | No foreign particles or crystals | No foreign particles or crystals |
| | observed. | observed. |
| | Observations Under 40x Objective | Observations Under 40x Objective |
| | (white light and polarized light): | (white light and polarized light): |
| | No foreign particles or crystals | No foreign particles or crystals |
| | observed. | observed. |
| pH | 5.2 | 5.2 |
| Assay (% LC) | Sample 1: 101.9% LC | Sample 1: 103.0% LC |
| | Sample 2: 100.9% LC | Sample 2: 100.7% LC |
| | Average: 101.4% LC | Average: 101.9% LC |
| Microbial Limits | <10 CFU/g (Dilution-1:10) | <10 CFU/g (Dilution-1:10) |
| (USP) | <10 CFU/g (Dilution-1:10) | <10 CFU/g (Dilution-1:10) |
| Test for Specified | Absent in 1 g (Dilution-1:10) | Absent in 1 g (Dilution-1:10) |
| Microorganisms | Absent in 1 g (Dilution-1:10) | Absent in 1 g (Dilution-1:10) |
| (USP) | | |

Table 14 demonstrates that the 0.0300 w/w and 0.100 w/w compositions are stable for at least 3 months. In particular, there is no changes in the pH or the assay of the compositions after 3 months storage.

### Example 11: Human Primary Keratinocytes Proliferation and Apoptosis Assays

### Human Primary Keratinocytes Proliferation Assay

The objective of this study was to determine a dose response for Compound A mesylate salt in which the compound regulates cell proliferation in human keratinocytes.

### Method:

Primary human keratinocytes (KMF112410A) were grown in 96-well plates and exposed to different concentration of Compound A mesylate salt, 0.1 nM to 100 μM, for 24 hours or 72 hours. Cell proliferation inhibition was evaluated by Bromodeoxyuridine (BrDU) incorporation and briefly, for cell proliferation, human primary neonatal keratinocytes were cultured in keratinocyte growth and plating medium (Zen-Bio KM-3) at a concentration of 5,000 cells/well in clear bottom 96-well plates overnight. The next morning, growth medium was removed and replaced with 50% supplemented keratinocyte growth medium containing compounds and controls.

After 24 or 72 hours treatment, 20 μL of the prepared 10×BrdU solution (Cell Signaling Technologies, catalog number 6813) was added to each of the wells and allowed to incubate at 37° C., 5% CO₂ for 4 hours. After 4 hours, the medium was removed and 100 μL of the Fixing/Denaturing solution (paraformaldehyde) was added to each well and allowed to incubate at room temperature for 30 minutes. After 30 minutes, the fixing solution was removed and 100 μl of the prepared 1× detection antibody (Cell Signaling Assay Kit, Catalogue No. 6813) was added to each well and allowed to incubate at room temperature for 1 hour. The plate was then washed 3 times with prepared wash buffer (details of wash buffer; PBS saline).

After washing, 100 μL of the prepared 1× Horseradish Peroxidase (HRP)-conjugated secondary antibody solution was added to each well and allowed to incubate at room temperature for 30 minutes. The plate was then washed 3 times with prepared wash buffer.

After washing the 100 μL 3,3′,5,5′-Tetramethylbenzidine (TMB) substrate was added to each well and incubated 30 minutes at room temperature. After 30 minutes, 100 μL of stop solution (2% paraformaldehyde) was added to each well and the absorbance was read at 450 nm using a microplate Reader SpectraMax iD3 (Molecular Devices). The magnitude of the absorbance for the developed color is proportional to the quantity of BrdU incorporated into cells, which is a direct indication of cell proliferation, and negative control (50% supplemented keratinocyte growth medium alone) was used as reference to evaluate cell proliferation inhibition (levels <100%) or activation (levels >100%).

### Results:

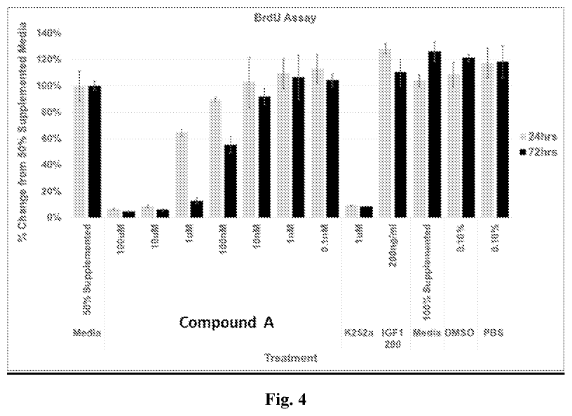

The results shown in FIG. 4 demonstrate that Compound A mesylate salt blocks proliferation in a dose-response matter starting to show significant inhibition at dose of 100 nM and higher after 24 hours of treatment and 10 nM and higher after 72 h treatment. K252a, which is a well-known TRKa inhibitor, was used as reference and positive control at a single concentration of 1 μM for cell proliferation inhibition. Insulin Growth Factor (IGF) 200 ng/mL was used as positive control for cell proliferation activation.

Using these results, a calculated potency (IC₅₀) of Compound A mesylate salt to block cell proliferation was determined to be 1.25 μM and 0.097 μM at 24 and 72 hours, respectively.

### Human Primary Keratinocytes Apoptosis Assay

The objective of this study was to determine a dose response for Compound A mesylate salt inducing apoptosis in human keratinocyte. Cell death/apoptosis was determined by Propidium Iodide (PI) and Annexin V staining.

### Method:

For the cell death/apoptosis, human primary neonatal keratinocytes were cultured (5,000 cells/well) in clear bottom 96-well plates overnight. The next morning, growth medium was removed and replaced with 50% supplemented keratinocyte growth medium containing compounds and controls.

After 24 or 72 hours treatment, 200 μL of the prepared 1:100 Annexin V (Biovision, catalog number K101) and PI in 1× binding buffer solution was added to each of the wells and allowed to incubate at room temperature for 5 minutes. Cells were then washed with DPBS (Dulbecco's Phosphate-Buffered Saline) and fixed for 30 minutes in 2% Paraformaldehyde (PFA). Following fixation, cells were washed 3× with 200 μL of DPBS. A final volume of 100 μL of DPBS were added to the wells. Quantification of Annexin V and PI were assessed using the Array Scan High Content Imaging System (ThermoFisher).

### Results:

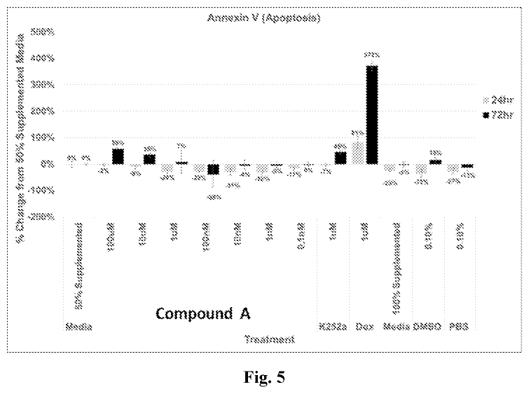

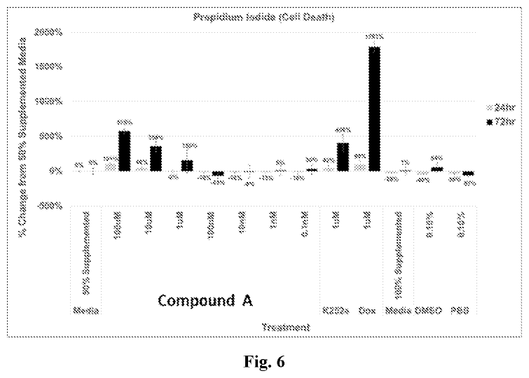

The signal quantified for Annexin V, an early apoptosis event marker (FIG. 5) and PI to assess cell death (apoptosis and necrosis) was compared to negative control (50% supplemented media) to determine the percentage of viability (FIG. 6). Doxorubicin (Dox) 1 μM was used as a positive control for inducing apoptosis, K252a as a comparator compound, and both showed significant cell death at their respective concentration after 24 and 72 hours of exposure. Significant cell death was observed at Compound A mesylate salt concentrations of 10 μM and 100 μM after 24 hours of treatment and 1 μM, 10 μM, and 100 μM after 72 hours of treatment.

These results show that the doses that human keratinocytes are exposed is critical to determine whether cell proliferation inhibition occurs alone or cell death is also induced.

In summary, the data shows that Compound A or a pharmaceutical salt thereof, is able to inhibit cell proliferation of keratinocytes, a key feature in inflammatory skin disorders, such as psoriasis. Furthermore, the data also provides an indication of a target concentration range at which hyper proliferation of keratinocytes can be inhibited but apoptosis of normal skin cells can be avoided.

### Example 12: Toxicology Summary

Toxicity evaluation of Compound A mesylate salt has comprised range-finding and pivotal repeat dose dermal and oral toxicology studies in minipigs, rats and dogs. The dermal route of administration was chosen because it is the intended to be the human therapeutic route. As per International Council for Harmonisation (ICH) guidelines, the oral route was selected in rats and dogs to assess systemic toxicity and to support dermal administration. The minipig was selected as the species for formal dermal toxicology testing of Compound A mesylate salt.

To support dermal administration of Compound A mesylate, in addition to oral repeat-dose toxicity studies in rats and dogs, dermal toxicity studies in minipigs were conducted. A 28-day repeat dose toxicity study was conducted by dermal application of Compound A mesylate salt at doses in the range of 0.1 mg/kg to 1 mg/kg.

To understand the relationship between potential Compound A mesylate exposure and toxicity (clinical observations), a non-GLP intravenous study was conducted. In this study, minipigs were administered up to 10 mg/kg daily for 7 days by intravenous administration to determine Compound A mesylate exposure and clinical observations. Overall, minipigs tolerated intravenous dose of up to 10 mg/kg/day for 7 seven days without any adverse clinical signs.

The 28-day dose toxicity study was initiated at doses in the range of 0.1 to 1.0 mg/kg. The dermal application of Compound A mesylate to Gottingen Minipigs at 0.1, 0.3, and 1 mg/kg/day for 28 consecutive days was well tolerated at all dose levels, without any adverse signs of toxicity. The NOAEL was determined to be the high dose, 1 mg/kg/day (Cₘₐₓ: 3.03, and 2.52 ng/mL for males and females respectively; AUC_{0-Tlast}: 32.6 and 37.2 hr*ng/mL for males and females respectively on Day 28). When compared to intravenous administration, the systemic exposure in minipigs was significantly lower after dermal administration.

In dog oral toxicity studies, Compound A mesylate was generally well tolerated with episodes of emesis and soft/fluid feces. There were no compound-related effects on body weight, food consumption, clinical pathology, or histopathology. The NOAEL for the one-month study was 2 mg/kg/day.

In a one-month rat study, Compound A mesylate caused a marked increase in food consumption and body weight gain at all dose levels. Histopathologically, there was myocardial inflammation, fibrosis and increased plasma phosphate and Troponin I in a minority of animals. The NOAEL for these findings was 0.5 mg/kg/day.

In general, systemic exposure in minipigs was low after repeat dermal administration compared to oral administration in rats and dogs. No notable gender differences in exposure were observed.

Compound A mesylate was non-irritant for erythema and edema in rabbits, was not considered to be contact sensitizers in guinea pigs and did not induce ocular irritation in bovine corneal opacity and permeability (BCOP) assay.

Combined with the data from Example 11, the data in this study indicates that there is a unique therapeutic window for Compound A, or a pharmaceutically acceptable salt thereof when applied topically. Indeed, a target dosing range at which hyper-proliferation of keratinocytes can be inhibited but apoptosis and adverse events can be avoided appears to exist. Doses outside of this target range are less optimal.

### Example 13: Preparation of 0.01%, 0.03% and 0.1% Formulations that were Used in Healthy Participants Cumulative Irritant Patch Test Trial

| | Composition | | | | Quality |
|---|---|---|---|---|---|
| Ingredient | (% w/w) | | | Purpose | Standard |
| Compound A mesylate salt | 0.01 | 0.03 | 0.10 | Active | In House |
| (Form B | | | | | |
| Transcutol HP | 5.00 | 5.00 | 5.00 | Permeation enhancer/Co- | NF |
| | | | | solvent | |
| Oleyl Alcohol | 3.00 | 3.00 | 3.00 | Permeation enhancer | NF |
| Octyldodecanol | 3.00 | 3.00 | 3.00 | Permeation enhancer | NF |
| Cyclomethicone | 3.00 | 3.00 | 3.00 | Counter irritant/Anti tack | NF |
| | | | | agent | |
| White petrolatum | 15.00 | 15.00 | 15.00 | Emollient | NF |
| Cetyl alcohol | 3.00 | 3.00 | 3.00 | Emulsifier | NF |
| Stearyl Alcohol | 3.00 | 3.00 | 3.00 | Emulsifier | NF |
| Glyceryl mono stearate Type | 7.00 | 7.00 | 7.00 | Emulsifier | NF |
| II | | | | | |
| Span 80 | 3.00 | 3.00 | 3.00 | Emulsifier | NF |
| Polysorbate 80 | 8.00 | 8.00 | 8.00 | Emulsifier | NF |
| Butylated hydroxy Toluene | 0.10 | 0.10 | 0.10 | Antioxidant | USP |
| Sorbic acid | 0.10 | 0.10 | 0.10 | Oil Phase Preservative | NF |
| Methyl Paraben | 0.18 | 0.18 | 0.18 | Aqueous Phase | USP |
| | | | | Preservative | |
| Propyl paraben | 0.02 | 0.02 | 0.02 | Preservative | USP |
| Disodium Edetate | 0.05 | 0.05 | 0.05 | Chelating agent | USP |
| Trolamine (25% diluted) QS | 0.01 | 0.01 | 0.01 | pH adjusting agent | Ph Eur |
| to pH 5.5 | | | | | |
| Purified Water | 46.53 | 46.51 | 46.44 | Solubilizer/Diluent | USP |

### General Manufacturing Process for Formulations at 1.5 Kg Scale:

The process below describes the preparation of a placebo formulation. Formulations comprising the API at 0.01, 0.03 and 0.1% w/w can be prepared in a similar manner wherein the amount of water is reduced in the composition to compensate for the amount of API added. The API is added to purified water to make a slurry and this slurry is added to the process at step 4.

Step 1: The oil phase is prepared in an Axomix vessels which is loaded with the following components:
a. Glyceryl monostearate—target amount 105 g
b. Cetyl alcohol—target amount 45 g
c. Stearyl alcohol—target amount 45 g
d. Heat to 70±5° C. and mix at 80 RPM until all components have melted
e. Add white petrolatum—target amount 225 g
f. Butylated hydoxy toluene—target amount 1.5 g
g. Sorbic acid—target amount 1.5 g
h. Propyl paraben—target amount 0.3 g
i. SPAN 80—target amount 45 g
j. Oleyl alcohol—target amount 45 g
k. Octyldodecanol—target amount 45 g
l. Cyclomethicone—target amount 45 g
m. Engage mixing for 10 min at 80 RPM at 70±5° C. and then homogenization at 2000 RPM for 10 min under vacuum (−0.4 to −0.6 Bar) maintaining at 70±5° C.

Step 2. The aqueous phase is prepared in a separate stainless steel vessel equipped with a hot plate and an IKA overhead stirrer. The following components are added to the stainless steel vessel
a. Purified water (Part-I)—target amount 698.1 g. Second water portion was used to make API slurry for easier, safer transfer which includes a rinse.
b. Polysorbate 80, NF—target amount 120 g
c. Methyl paraben—target amount 2.7 g
d. Disodium edetate, NF—target amount 0.75 g
e. Stir until a clear solution is obtained

Step 3. To the aqueous phase add transcutol HP—target amount 75 g. Heat the mixture with stirring (80 RPM) to a temperature of 60±2° C.

Step 4. Once both the oil and aqueous phases reach temperature of 60-65° C., add the aqueous phase to the oil phase in the anomix mixer. Mix at 80 RPM during addition stage and then increase mixing rate to 2000 RPM for 30 min.

Step 5. Open anomix mixer after 20 min of mixing and check for product appearance. A white milky emulsion is desired. If watery layer is observed, continue mixing at 80 RPM and homogenization at 2000 RPM for 10 min. Repeat visual inspection. Continue until desired emulsion observed. Do not exceed 50 min of total mixing time.

Step 6. Cool the product to 30° C. (28±2° C.) at 1° C./minute.

Step 7. The pH of the product is adjusted with the addition of trolamine—target amount 0.15 g. pH is checked as an in-process test. Samples are taken from the bulk, tested, then additional Trolamine is added, if needed. pH target range is 5.5±0.5 units.

Step 8. Transfer product to HDPE Bulk holding containers under nitrogen purge.

### Example 14: Evaluation of Formulations 0.01%, 0.03% and 0.1% Formulations in Healthy Participants Using a Cumulative Irritant Patch Test Design

### Study Objective:

The objective of this study was to assess the primary irritancy potential of Compound A mesylate Topical Cream in human skin using a Repeat Insult Patch Testing (RIPT) over twenty-one (21) days.

### Study Design:

This RIPT was an open-label, single-arm (cell), randomized, evaluator-blinded study wherein test products were applied under an occlusive dressing to the upper back or upper arm continuously and repeatedly to the same site for a period of 21 days. Three concentrations of The Cream described in Example 13 (0.1%, 0.03%, 0.01%) and Vehicle (Placebo Cream—0%) were applied to the skin of healthy subjects aged 18-70 with a Fitzpatrick Skin Type of I-III. In addition, one site was treated with 0.4 ml of 0.25% sodium lauryl sulfate (SLS) as a positive control and another site was treated with a plain Webril patch (cotton cloth) which served as a negative control. The total daily dose of Compound A received by each subject was 560 μg (0.4 ml of each of the three concentrations/3 different sites) or approximately 8 μg/kg. The evaluator graded the level of irritation for each test site at every study visit and was blinded as to the identity of the test products. The targeted sample size was at least 25 subjects (n=25) at completion.

### Study Schedule:

Below is the schedule of events (per protocol) that occurred during the study:

| | M | T | W | Th | F | S | S | M | T | W | Th | F | S | S | M | T | W | Th | F | S | S | M |
|---|---|---|---|---|---|---|---|---|---|---|---|---|---|---|---|---|---|---|---|---|---|---|
| Day: | 0 | 1 | 2 | 3 | 4 | 5 | 6 | 7 | 8 | 9 | 10 | 11 | 12 | 13 | 14 | 15 | 16 | 17 | 18 | 19 | 20 | 21 |
| Test Material | X | X | X | X | X | | | X | X | X | X | X | | | X | X | X | X | X | | | \hat{ } |
| Application* | | | | | | | | | | | | | | | | | | | | | | |

*Test site graded, and test material reapplied
\hat{ }Final skin grading

For inclusion into this study, each subject fulfilled all the following criteria:
Healthy adult male and female volunteers between the ages of 18 and 70 years with Fitzpatricktypes I, II, or III
   Type I—Always burns easily, never tans
   Type II—Always burns easily, tans minimally
   Type III—Burns moderately, tans gradually
Willing to follow the study requirements and voluntarily gave their informed consent
Was able to read and follow study instructions in English
Generally in good health as determined by the investigator (based on medical history interview)
Evidence of a personally signed and dated Informed Consent indicating that the subject had been informed of and understood all pertinent aspects of the trial.

The following were regarded as criteria for excluding a subject from the study:
Scars, moles, or other blemishes over the forearm, upper arm, or back that interfered with the study
Sunburn within the last three weeks or use of tanning beds
History of allergy or hypersensitivity to skin care or consumer products including fragrances, cosmetics, toiletries, or any kind of tape
History of chronic or recurrent dermatological diseases, e.g., psoriasis, atopic eczema, chronic urticaria
Subjects receiving systemic or topical drugs which could have interfered with the development of an inflammatory response, e.g., steroids, immunosuppressive agents, or retinoids
History of any significant systemic diseases e.g., cardiac, pulmonary, renal, hepatic, etc. that could have impacted subject's ability to complete the study
Pregnancy or mothers who were breastfeeding or planning a pregnancy
Other conditions considered by the Investigator as sound reasons for disqualification from enrollment into the study.

### Study Restrictions:

While participating on this study, subjects were asked not to:
Participate in any other clinical study for the duration of this test
Use lotions or medications on the test sites
Take more than 8 pain relievers per day (examples: ibuprofen, aspirin)
Take any over-the-counter antihistamines (example: Benadryl, cold medicines) for the duration of the study
Expose back to sunlight (no sunbathing) or use of tanning beds
Swim during the course of the study.

### Study Visit Procedures:

### Day 0 to Day 21

Approximately 0.4 ml of each test material was spread uniformly onto a 15 mm diameter circular disc of non-absorbing cotton cloth (Webril) using micropipettes or plastic tuberculin syringes. The treated circular disc was applied to the designated skin site (see Randomization Section below) measuring 15 mm in diameter on the upper arm or upper back. In addition to the test products, one site was treated with 0.4 ml of 0.25% sodium lauryl sulfate (SLS) as a positive control and another site was treated with a plain Webril patch (cotton cloth) as a negative control. Each site was covered with an occlusive tape (Blenderm, 3M) and the entire patch fastened to the skin with Scanpor or Hypafix Tape to ensure intimate contact with the skin. The initial patch was applied on Day 0 and grading began on Day 1.

Subjects returned to the testing facility once daily (Monday to Friday). At each visit, the patches were removed, and the test sites graded by the Principal Investigator (PI) for irritation or inflammation. This was followed by fresh reapplications of the test materials and occlusive patches. This procedure was repeated daily Mondays through Fridays for three consecutive weeks, with patches remaining in place over the weekends (Friday to Monday). Subjects were questioned and assessed for adverse events as well as any changes in their health or concomitant medications at every study visit. Final grading of skin sites by the PI occurred on Day 21.

### Assessment and Grading of Skin Irritation

All patches were inspected at each study visit and adhesion issues were noted when present. Patch sites were inspected and irritant reactions provoked during the study were recorded daily (Monday-Friday). All test sites were graded using the following ICDRG scale [Ivens U, O'Goshi K, Serup J. Allergy patch test reading from photographic images: disagreement on ICDRG grading but agreement on simplified tripartite reading. Skin ResTechnol. 2007 February; 13(1):110-3.]:
0=no visible redness
1=faint redness, poorly defined margins
2=moderate redness, well defined margins
3=intense redness
4=caustic erythema—erosive and/or necrotic aspect

### Other Notations:

V=Vesicles
E=Erosions
F=Fissuring
   Test sites achieving a grade 3 or greater score were discontinued, and that grade was carried forward for the
   remainder of the test days for the purpose of calculating the cumulative irritation index.

### Cumulative Irritation Index (CII) and Irritancy Potential

The CII was calculated for each product as well as the positive and negative control sites. The CII is the ratio calculated by dividing the sum of the cumulative irritation scores (SCS) from all subjects by the number of subjects in the completed panel×the maximum tolerated score per patch site (3)×the number of grading assessments (15).

The range of the CII scores is correlated to the Irritancy Potential as below:

| CII Scoring | Irritancy Potential |
|---|---|
| 0.0-0.06 | negligible or no significant irritation |
| 0.07-0.15 | minimal or weak irritancy potential |
| 0.16-0.22 | mild irritancy potential |
| 0.23-0.33 | moderate irritancy potential |
| 0.34-0.55 | severe irritancy potential |

### Randomization and Blinding

The order of test product patch placement on each subject's upper back or upper arm was randomized. The randomization schedule was done in blocks to balance the treatments. The randomization was assigned in ascending numerical order during screening. Each test product was graded individually along with the positive and negative control. The PI graded the level of irritation at each test site and was blinded as to the identity of the test products. Personnel applying the study did not participate in the evaluation of subjects in order to minimize potential bias. To maintain evaluator blindness, visual assessments were conducted in an area away from product application.

### Pharmaceutical Compositions:

The pharmaceutical compositions described in Example 13 were used in the study.

Placebo contained the same basic non-medicinal ingredients as the pharmaceutical composition described above.

### Results:

### Demographic Data:

Nine (9) male and 21 female Caucasian subjects (Fitzpatrick Skin Type I-III), between the ages of 18 to 68 years, were enrolled.

### Adverse Events:

Four (4) Adverse Events (AEs) occurred during this study; none were product-related. There were no Serious Adverse Events.

### Cumulative Irritation Index (CII) and Irritancy Potency Results

Below are the analysis tables of the irritancy potential of the test products observed during this study by the Principal Investigator.

0.1% Cream

| | |
|---|---|
| Sum of Cumulative Scores | 0 |
| Mean Cumulative Irritation Score | 0.00 |
| Mean Daily Score | 0.00 |
| Cumulative Irritation Index (CII) | 0.00 |

0.03% Cream

| | |
|---|---|
| Sum of Cumulative Scores | 1 |
| Mean Cumulative Irritation Score | 0.04 |
| Mean Daily Score | 0.00 |
| Cumulative Irritation Index (CII) | 0.00 |

0.01% Cream

| | |
|---|---|
| Sum of Cumulative Scores | 0 |
| Mean Cumulative Irritation Score | 0.00 |
| Mean Daily Score | 0.00 |
| Cumulative Irritation Index (CII) | 0.00 |

Placebo Cream

| | |
|---|---|
| Sum of Cumulative Scores | 1 |
| Mean Cumulative Irritation Score | 0.04 |
| Mean Daily Score | 0.00 |
| Cumulative Irritation Index (CII) | 0.00 |

Webril Cotton (Negative Control)

| | |
|---|---|
| Sum of Cumulative Scores | 0 |
| Mean Cumulative Irritation Score | 0.00 |
| Mean Daily Score | 0.00 |
| Cumulative Irritation Index (CII) | 0.00 |

0.25% Sodium Lauryl Sulfate (Positive Control)

| | |
|---|---|
| Sum of Cumulative Scores | 294 |
| Mean Cumulative Irritation Score | 10.89 |
| Mean Daily Score | 0.73 |
| Cumulative Irritation Index (CII) | 0.24 |

### SUMMARY

The enrollment goal was exceeded and there were no product-related adverse events observed during the study. Positive and negative controls performed as expected. Under the above-described test conditions, the creams were all found to possess no significant irritation potential in human skin.

### EQUIVALENTS

It is to be understood that the invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A topical pharmaceutical composition comprising:
a) Compound A or a pharmaceutically acceptable salt thereof: 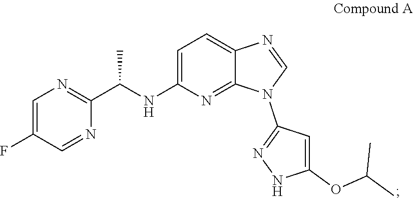 [SMILES: CC(C)Oc1cc(-n2cnc3ccc(N[C@@H](C)c4ncc(F)cn4)nc32)n[nH]1]
b) at least one permeation enhancer; and
c) one or more pharmaceutically acceptable excipients.

2. The composition according to claim 1, wherein the pharmaceutical composition is a cream, an ointment, a paste, a solution, a lotion, a gel, a foam or a spray.

3. The composition according to claim 1, wherein the pharmaceutical composition is a cream.

4. The composition according to any preceding claim, wherein Compound A, or a pharmaceutically acceptable salt thereof, is soluble in at least one permeation enhancer present within the composition.

5. The composition according to any one of claims 1 to 4, comprising one or more permeation enhancers selected from diethylene glycol monoethyl ether, a fatty acid alcohol, a glycol, an aprotic solvent, or any combination thereof.

6. The composition according to claim 1, wherein the composition comprises at least two permeation enhancers and one of these permeation enhancers is diethylene glycol monoethyl ether.

7. The composition according to claim 1, comprising a first permeation enhancer that is diethylene glycol monoethyl ether, a second permeation enhancer that is a fatty acid alcohol and optionally a third permeation enhancer that is also a fatty acid alcohol.

8. The composition according to claim 6, wherein the second permeation enhancer is oleyl alcohol and the optional third permeation enhancer is octyldodecanol.

9. The composition according to claim 8, wherein the weight ratio of the diethylene glycol monoethyl ether to oleyl alcohol is from about 1:2 to about 6:1, conveniently from about 1:1 to about 4:1.

10. The composition according to any one of claims 5 to 9 wherein the diethylene glycol monoethyl ether is present at 3 to 40% w/w of the total composition.

11. The composition according to any one of claims 5 to 9, wherein the diethylene glycol monoethyl ether is present at 3 to 10% w/w of the total composition.

12. The composition according to any one of the preceding claims, wherein the composition further comprises an antioxidant.

13. The composition according to claim 12, wherein the antioxidant comprises butylated hydroxytoluene (BHT), butylated hydroxyanisole (BHA), propylgallate, squalene, D-alpha tocopherol or D-alpha tocopherol acetate, or a combination thereof.

14. The composition according to claim 12, wherein the antioxidant is selected from butylated hydroxytoluene (BHT), butylated hydroxyanisole (BHA), or a combination thereof.

15. The composition according to claim 12, wherein the antioxidant is butylated hydroxytoluene (BHT).

16. The composition according to any one of claims 12 to 15, wherein the antioxidant is present in the composition from about 0.01% to about 1.0% w/w of the total composition.

17. The composition according to any preceding claim, wherein the composition comprises diethylene glycol monoethyl ether, oleyl alcohol and an oil-soluble antioxidant.

18. The composition according to any preceding claim, wherein the composition comprises between 0.005% w/w and 5% w/w (conveniently between 0.005% w/w and 3% w/w, and more conveniently between 0.005% w/w and 0.15% w/w) of Compound A or a pharmaceutically acceptable salt thereof.

19. The composition according to any preceding claim, wherein the composition comprises a pharmaceutically acceptable salt of Compound A.

20. The composition according to claim 19, wherein the pharmaceutically acceptable salt is a mesylate salt of Compound A.

21. The composition according to any preceding claim, wherein the composition comprises a pH adjusting agent that is a weak organic base.

22. The composition according to claim 21, wherein the pH adjusting agent is selected from triethanolamine, tromethamine (Tris; tris(hydroxymethyl)aminomethane), di-isopropyl amine, and trometamol, or a combination thereof

23. The composition according to 21 or 22, wherein the molar ratio of the pH adjusting agent to Compound A is from 2:1 to 1:4.

24. The composition according to any one of the preceding claims, wherein the composition is a cream and is physically stable to phase separation when stored at 25° C. and 60% RH for at least about 1 month.

25. The composition according to any one of the preceding claims, wherein the composition is a cream based on an oil-in-water emulsion and the droplet size of the oil ranges from 1 μm to 50 μm, conveniently between 1 μm to 30 μm.

26. The composition according to any one of the preceding claims, wherein the composition is a cream, and wherein the pH of the cream is in the range of about 3.5 to about 7.0, conveniently in the range from about 4.5 to about 6.5.

27. The composition according to any one of the preceding claims, wherein the composition is a cream and has an overall HLB value in the range of about 7 to about 12.

28. The composition according to any one of the preceding claims, wherein the composition provides less than 5% (conveniently less than 3%) chemical degradation of Compound A or a pharmaceutically acceptable salt thereof by HPLC when stored at 25° C. and 60% RH for 1 month.

29. The composition according to any one of the preceding claims, wherein when the topical pharmaceutical composition is subject to an in-vitro permeation test the topical composition provides a flux of less than 5 ng/cm²·h.

30. The composition according to any one of the preceding claims, wherein the area under the plasma concentration-time curve from time zero up to 24 hours post-dose (AUC₀₋₂₄) after the composition is administered topically to the skin of the subject in a single application is less than approximately 100 ng·hr/mL.

31. A method of treating or preventing an inflammatory skin disorder comprising administration of a topical pharmaceutical composition to a subject in need thereof, wherein the topical pharmaceutical composition comprises Compound A: 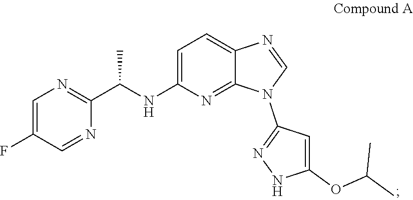 [SMILES: CC(C)Oc1cc(-n2cnc3ccc(N[C@@H](C)c4ncc(F)cn4)nc32)n[nH]1]
or a pharmaceutically acceptable salt thereof;
and wherein the composition is:
a) applied topically to an affected area of the skin of a subject at an application dose of Compound A or a pharmaceutically acceptable salt thereof in the range of 0.05 μg/cm² and 1000 μg/cm² skin surface; and
b) the composition is administered in an amount sufficient to prevent or treat the inflammatory skin disorder in the subject.

32. The method according to claim 31, wherein the composition comprises from about 0.001% w/w to about 5% w/w of Compound A, or a pharmaceutically acceptable salt thereof, conveniently between 0.01% w/w and 0.1% w/w of Compound A, or a pharmaceutically acceptable salt thereof.

33. The method according to claim 31, wherein the composition comprises between 0.001% w/w and 5% w/w of Compound A, or a pharmaceutically acceptable salt thereof and the amount of said composition administered topically to the skin of the subject per application ranges from about 0.05 mg to about 1 g.

34. The method according to claims 31 to 33, wherein the composition is administered to the subject in need thereof between once and five times daily, conveniently once daily.

35. The method according to claims 31 to 34, wherein the total dose of Compound A, or a pharmaceutically acceptable salt thereof administered topically to the skin of the subject in a single day ranges from about 0.05 mg to about 2 g.

36. The method according to claims 31 to 35, wherein the concentration of the Compound A, or a pharmaceutically acceptable salt thereof achieved within the epidermis at 24 hours after administration is between 50 nM and 1.5 μM, more conveniently 100 nM and 1.0 μM.

37. The method according to claims 31 to 36, wherein the inflammatory skin disorder is associated with elevated expression or activity of a tropomyosin-related kinase (TRK).

38. The method according to claims 31 to 37, wherein the inflammatory skin disorder is selected from psoriasis, actinic keratosis, psoriasis guttata, inverse psoriasis, pustular psoriasis, psoriatic erythroderma, acute febrile neutrophilic dermatosis, eczema, xerotic eczema, dyshidrotic eczema, vesicular palmar eczema, acne vulgaris, atopic dermatitis, contact dermatitis, allergic contact dermatitis, dermatomyositis, exfoliative dermatitis, hand eczema, pompholyx, keloids, rosacea, rosacea due to sarcoidosis, rosacea due to scleroderma, rosacea due to Sweet syndrome, rosacea due to systemic lupus erythematosus, rosacea due to urticaria, rosacea due to herpetic pain, mastocytosis, uticaria, Sweet's disease, neutrophilic hydrodenitis, sterile pustule, drug rash, seborrheic dermatitis, Pityriasis rosea, Kikuchi's disease of the skin, pruritic urticarial papules and plaques of pregnancy, Stevens-Johnson syndrome and toxic epidermal necrolysis, tattoo reaction, Wells syndrome (eosinophilic cellulitis), reactive arthritis (Reiter syndrome), bowel-associated dermatosis-arthritis syndrome, rheumatoid neutrophilic dermatosis, neutrophilic eccrine hidradenitis, neutrophilic skin disease of dorsum of hand, balanitis circumscripta plasmacellularis, balanoposthitis, Behcet's disease, erythema annulare centrifugum, erythema dyschromicum perstans, erythema multiforme, granuloma annulare, dermatitis of hand, Lichen nitidus, Lichen planus, Lichen sclerosus et atrophicus, Lichen simplex chronicus, Lichen spinulosus, nummular dermatitis, pyoderma gangrenosum, sarcoidosis, subcorneal pustular dermatosis, urticaria, transient acantholytic dermatosis, bullous pemphigoid, dermatitis herpetiformis, dermatophytic infections, folliculitis, pediculosis, scabies, oral Lichen planus and aphthous ulcers.

39. The method according to claims 31 to 37, wherein the inflammatory skin disorder is psoriasis.

40. The method according to claim 39, wherein the psoriasis is selected from plaque psoriasis, guttate psoriasis, inverse psoriasis, mild to moderate psoriasis, moderate to severe psoriasis, pustular psoriasis and erythrodermic psoriasis.

41. The method according to claims 31 to 40, wherein the inflammatory skin disorder is psoriasis and administration of the composition is well tolerated with no dermal irritation or only minimal erythema.

42. The method according to claims 31 to 41, wherein the pharmaceutical composition is a cream.

43. The method according to claims 31 to 42, wherein the pharmaceutical composition comprises Compound A mesylate salt.

44. The method according to claims 31 to 43 wherein the method comprises administration of the topical pharmaceutical composition according to claims 1 to 30.
